SEQUENCE LISTING (137 sequences)

SEQ ID NO: 1 - protein, 5 aa, synthetic construct
  REGION 1..5: note = "Synthetic peptide: CDR1 of 3h-56-269"
DYEMW

SEQ ID NO: 2 - protein, 17 aa, synthetic construct
  REGION 1..17: note = "Synthetic peptide: CDR2 of 3h-56-269"
AINPQGTRTYYADSVKG

SEQ ID NO: 3 - protein, 7 aa, synthetic construct
  REGION 1..7: note = "Synthetic peptide: CDR3 of 3h-56-269"
LPFRFSD

SEQ ID NO: 4 - protein, 5 aa, synthetic construct
  REGION 1..5: note = "Synthetic peptide: CDR1 consensus"
  REGION 1..5: note = MISC_FEATURE - "Variant residues given in the sequence have no preference with respect to those in the annotations for variant positions"
  VARIANT 1: note = Asp or Gly
  VARIANT 4: note = Met or Leu
DYEXW

SEQ ID NO: 5 - protein, 17 aa, synthetic construct
  REGION 1..17: note = "Synthetic peptide: CDR2 consensus"
  REGION 1..17: note = MISC_FEATURE - "Variant residues given in the sequence have no preference with respect to those in the annotations for variant positions"
  VARIANT 16: note = Lys or Met
  VARIANT 8: note = Arg, Leu, Tyr, His, or Phe
  VARIANT 5: note = Gln, Tyr, His, Trp, or Ala
  VARIANT 7: note = Thr, Asn, Gly, Ser, or Gln
AINPXGXXTYYADSVXG

SEQ ID NO: 6 - protein, 7 aa, synthetic construct
  VARIANT 1: note = Leu, Pro, or Glu
  VARIANT 5: note = Phe or Tyr
  VARIANT 3: note = Phe, Gln, Thr, Met, or Tyr
  VARIANT 6: note = Ser, Gln, His, Asp, Lys, Glu, or Gly
  VARIANT 4: note = Arg, Tyr, Pro, Leu, Thr, Ile, Phe, Met, or Ser
  REGION 1..7: note = "Synthetic peptide: CDR3 consensus"
  VARIANT 7: note = Asp, Tyr, Glu, or Ser
  REGION 1..7: note = MISC_FEATURE - "Variant residues given in the sequence have no preference with respect to those in the annotations for variant positions"
LPFRXXX

SEQ ID NO: 7 - protein, 7 aa, synthetic construct
  REGION 1..7: note = "Synthetic peptide: CDR3-3h-56-1"
LPFTFED

SEQ ID NO: 8 - protein, 7 aa, synthetic construct
  REGION 1..7: note = "Synthetic peptide: CDR3-3H-56-2"
LPFTFDD

SEQ ID NO: 9 - protein, 7 aa, synthetic construct
  REGION 1..7: note = "Synthetic peptide: CDR3-3h-56-202"
LPTYFSD

SEQ ID NO: 10 - protein, 7 aa, synthetic construct
  REGION 1..7: note = "Synthetic peptide: CDR3-3h-56-205"
LPFYFSE

SEQ ID NO: 11 - protein, 7 aa, synthetic construct
  REGION 1..7: note = "Synthetic peptide: CDR3-3h-56-207"
LPFPFSE

SEQ ID NO: 12 - protein, 7 aa, synthetic construct
  REGION 1..7: note = "Synthetic peptide: CDR3-3h-56-7"
PPFIFGD

SEQ ID NO: 13 - protein, 7 aa, synthetic construct
  REGION 1..7: note = "Synthetic peptide: CDR3-3h-56-22"
LPFIFEY

SEQ ID NO: 14 - protein, 7 aa, synthetic construct
  REGION 1..7: note = "Synthetic peptide: CDR3-3h-56-201"
LPFYFQE

SEQ ID NO: 15 - protein, 7 aa, synthetic construct
  REGION 1..7: note = "Synthetic peptide: CDR3-3h-56-203"
LPFFFEE

SEQ ID NO: 16 - protein, 7 aa, synthetic construct
  REGION 1..7: note = "Synthetic peptide: CDR3-3h-56-204"
LPTYFKD

SEQ ID NO: 17 - protein, 7 aa, synthetic construct
  REGION 1..7: note = "Synthetic peptide: CDR3-3h-56-206"
LPMFFED

SEQ ID NO: 18 - protein, 7 aa, synthetic construct
  REGION 1..7: note = "Synthetic peptide: CDR3-3h-56-258"
LPFYFHE

SEQ ID NO: 19 - protein, 7 aa, synthetic construct
  REGION 1..7: note = "Synthetic peptide: CDR3-3h-56-261"
LPFFFQE

SEQ ID NO: 20 - protein, 7 aa, synthetic construct
  REGION 1..7: note = "Synthetic peptide: CDR3-3h-56-262"
LPQLFHD

SEQ ID NO: 21 - protein, 7 aa, synthetic construct
  REGION 1..7: note = "Synthetic peptide: CDR3-3h-56-265"
LPQLFQD

SEQ ID NO: 22 - protein, 7 aa, synthetic construct
  REGION 1..7: note = "Synthetic peptide: CDR3-3h-56-266"
LPFFFHE

SEQ ID NO: 23 - protein, 7 aa, synthetic construct
  REGION 1..7: note = "Synthetic peptide: CDR3-3h-56-270"
LPTLFQD

SEQ ID NO: 24 - protein, 7 aa, synthetic construct
  REGION 1..7: note = "Synthetic peptide: CDR3-3h-56-9"
LPFMFDD

SEQ ID NO: 25 - protein, 7 aa, synthetic construct
  REGION 1..7: note = "Synthetic peptide: CDR3-3h-56-23"
EPYSFDS

SEQ ID NO: 26 - protein, 7 aa, synthetic construct
  REGION 1..7: note = "Synthetic peptide: CDR3-3h-56-24"
EPYSYDY

SEQ ID NO: 27 - protein, 17 aa, synthetic construct
  REGION 1..17: note = "Synthetic peptide: CDR2-3h-56-16"
AINPQGTRTYYADSVMG

SEQ ID NO: 28 - protein, 17 aa, synthetic construct
  REGION 1..17: note = "Synthetic peptide: CDR2-3h-56-246"
AINPWGTLTYYADSVKG

SEQ ID NO: 29 - protein, 17 aa, synthetic construct
  REGION 1..17: note = "Synthetic peptide: CDR2-3h-56-215"
AINPHGSLTYYADSVKG

SEQ ID NO: 30 - protein, 17 aa, synthetic construct
  REGION 1..17: note = "Synthetic peptide: CDR2-3h-56-217"
AINPWGSLTYYADSVKG

SEQ ID NO: 31 - protein, 17 aa, synthetic construct
  REGION 1..17: note = "Synthetic peptide: CDR2-3h-56-220"
AINPYGGLTYYADSVKG

SEQ ID NO: 32 - protein, 17 aa, synthetic construct
  REGION 1..17: note = "Synthetic peptide: CDR2-3h-56-224"
AINPWGQLTYYADSVKG

SEQ ID NO: 33 - protein, 17 aa, synthetic construct
  REGION 1..17: note = "Synthetic peptide: CDR2-3h-56-232"
AINPWGSHTYYADSVKG

SEQ ID NO: 34 - protein, 17 aa, synthetic construct
  REGION 1..17: note = "Synthetic peptide: CDR2-3h-56-239"
AINPWGSLTYYADSVKG

SEQ ID NO: 35 - protein, 17 aa, synthetic construct
  REGION 1..17: note = "Synthetic peptide: CDR2-3h-56-243"
AINPWGGFTYYADSVKG

SEQ ID NO: 36 - protein, 17 aa, synthetic construct
  REGION 1..17: note = "Synthetic peptide: CDR2-3h-56-244"
AINPWGGLTYYADSVKG

SEQ ID NO: 37 - protein, 17 aa, synthetic construct
  REGION 1..17: note = "Synthetic peptide: CDR2-3h-56-253"
AINPAGGYTYYADSVKG

SEQ ID NO: 38 - protein, 17 aa, synthetic construct
  REGION 1..17: note = "Synthetic peptide: CDR2-3h-56-248"
AINPYGNLTYYADSVKG

SEQ ID NO: 39 - protein, 5 aa, synthetic construct
  REGION 1..5: note = "Synthetic peptide: CDR1-3h-56-31"
GYEMW

SEQ ID NO: 40 - protein, 5 aa, synthetic construct
  REGION 1..5: note = "Synthetic peptide: CDR1-3h-56-28"
GYELW

SEQ ID NO: 41 - protein, 116 aa, synthetic construct
  REGION 1..116: note = "Synthetic polypeptide: 3h-56-269"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSS

SEQ ID NO: 42 - protein, 30 aa, synthetic construct
  REGION 1..30: note = "Synthetic polypeptide: FR1 of 3h-56-269 (1-30 of SEQ ID NO: 41)"
EVQLLESGGGLVQPGGSLRLSCAASGFTFR

SEQ ID NO: 43 - protein, 30 aa, synthetic construct
  REGION 1..30: note = "Synthetic polypeptide: FR1"
EVQLLESGGGLVQPGGSLRLSCAASGFTFA

SEQ ID NO: 44 - protein, 14 aa, synthetic construct
  REGION 1..14: note = "Synthetic peptide: FR2 of 3h-56-269 (36-49 of SEQ ID NO: 41)"
WVRQAPGKGLERVS

SEQ ID NO: 45 - protein, 14 aa, synthetic construct
  REGION 1..14: note = "Synthetic peptide: FR2"
WVRRAPGKGLERVS

SEQ ID NO: 46 - protein, 14 aa, synthetic construct
  REGION 1..14: note = "Synthetic peptide: FR2"
WFRQAPGKGLERVS

SEQ ID NO: 47 - protein, 32 aa, synthetic construct
  REGION 1..32: note = "Synthetic polypeptide: FR3 of 3h-56-269 (67-98 of SEQ ID NO: 41)"
RFTISRDNSKNTLYLQMNSLRAEDTAVYYCAK

SEQ ID NO: 48 - protein, 32 aa, synthetic construct
  REGION 1..32: note = "Synthetic polypeptide: FR3"
RFTISRDNSKNMLYLQMNSLRAEDTAVYYCAK

SEQ ID NO: 49 - protein, 32 aa, synthetic construct
  REGION 1..32: note = "Synthetic polypeptide: FR3"
RFTISRDNSKNTLYLQMNSLRAEDTAVYHCAK

SEQ ID NO: 50 - protein, 32 aa, synthetic construct
  REGION 1..32: note = "Synthetic polypeptide: FR3"
RFTISRDNSKNTLYLQMNSLRAEDTAVYYCTK

SEQ ID NO: 51 - protein, 32 aa, synthetic construct
  REGION 1..32: note = "Synthetic polypeptide: FR3"
RFTISRDNSKNTLYLQMNSLRAEDTAVYYCLK

SEQ ID NO: 52 - protein, 32 aa, synthetic construct
  REGION 1..32: note = "Synthetic polypeptide: FR3"
RFTISRDNSKNTLYLQMNSLRAEDTSVYHCAK

SEQ ID NO: 53 - protein, 32 aa, synthetic construct
  REGION 1..32: note = "Synthetic polypeptide: FR3"
RFTISRDNSKNTLYLQMNSLRADDTAVYHCTK

SEQ ID NO: 54 - protein, 11 aa, synthetic construct
  REGION 1..11: note = "Synthetic peptide: FR4 of 3h-56-269 (106-116 of SEQ ID NO: 41)"
RGQGTLVTVSS

SEQ ID NO: 55 - protein, 11 aa, synthetic construct
  REGION 1..11: note = "Synthetic peptide: FR4"
WGQGTLVTVSS

SEQ ID NO: 56 - protein, 11 aa, synthetic construct
  REGION 1..11: note = "Synthetic peptide: FR4"
WGQGTLVTVSN

SEQ ID NO: 57 - skipped

SEQ ID NO: 58 - protein, 5 aa, synthetic construct
  REGION 1..5: note = "Synthetic peptide: Linker"
GGGGS

SEQ ID NO: 59 - protein, 10 aa, synthetic construct
  REGION 1..10: note = "Synthetic peptide: Linker (GGGGS)2"
GGGGSGGGGS

SEQ ID NO: 60 - protein, 15 aa, synthetic construct
  REGION 1..15: note = "Synthetic peptide: Linker (GGGGS)3"
GGGGSGGGGSGGGGS

SEQ ID NO: 61 - protein, 20 aa, synthetic construct
  REGION 1..20: note = "Synthetic peptide: Linker (GGGGS)4"
GGGGSGGGGSGGGGSGGGGS

SEQ ID NO: 62 - protein, 25 aa, synthetic construct
  REGION 1..25: note = "Synthetic peptide: Linker (GGGGS)5"
GGGGSGGGGSGGGGSGGGGSGGGGS

SEQ ID NO: 63 - protein, 6 aa, synthetic construct
  REGION 1..6: note = "Synthetic peptide: Linker"
TVAAPS

SEQ ID NO: 64 - protein, 277 aa, Homo sapiens
  source 1..277: note = CD40
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 65 - protein, 232 aa, synthetic construct
  VARIANT 141: note = Asp or Glu
  VARIANT 232: note = Lys or absent
  VARIANT 143: note = Leu or Met
  REGION 1..232: note = "Synthetic polypeptide: Fc- consensus"
  REGION 1..232: note = MISC_FEATURE - "Variant residues given in the sequence have no preference with respect to those in the annotations for variant positions"
  VARIANT 23: note = Lys, Ser, Ala, Arg or Trp
EPKSCDKTHTCPPCPAPELLGGXSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 66 - protein, 232 aa, synthetic construct
  REGION 1..232: note = "Synthetic polypeptide: Fc IgG1a-P238K"
EPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 232 aa, synthetic construct
  REGION 1..232: note = "Synthetic polypeptide: Fc IgG1f-P238K"
EPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 232 aa, synthetic construct
  REGION 1..232: note = "Synthetic polypeptide: Fc IgG1a-P238S"
EPKSCDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 232 aa, synthetic construct
  REGION 1..232: note = "Synthetic polypeptide: Fc IgG1a-P238S"
EPKSCDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 351 aa, synthetic construct
  REGION 1..351: note = "Synthetic polypeptide: 3h-56-269-Fc IgG1a-P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 351 aa, synthetic construct
  REGION 1..351: note = "Synthetic polypeptide: 3h-56-269-IgG1f-P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 348 bp, synthetic construct
  source 1..348: mol_type = other DNA
  misc_feature 1..348: note = "Synthetic polynucleotide: coding sequence for 3h-56-269"
gaggtgcagctgttggagtctgggggaggcttggtacagcctggggggtccctgcgtctctcctgtgcagcctccggattcacctttcgggattatgagatgtggtgggtccgccaggctccagggaagggtctagagcgggtctcagctattaatccgcagggtacgcgtacatactacgcagactccgtgaagggccggttcaccatctcccgcgacaattccaagaacacgctgtatctgcaaatgaacagcctgcgtgccgaggataccgcggtatattactgtgcgaaacttccgtttaggttttccgaccggggtcagggaaccctggtcaccgtctcgagc

SEQ ID NO: 73 - skipped

SEQ ID NO: 74 - protein, 7 aa, synthetic construct
  REGION 1..7: note = "Synthetic peptide: Linker"
ASTSGPS

SEQ ID NO: 75 - protein, 348 aa, synthetic construct
  REGION 1..348: note = "Synthetic polypeptide: 3h56-269-IgG4.1 (BMS-986090)"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 351 aa, synthetic construct
  REGION 1..351: note = "Synthetic polypeptide: 3h56-269-CT"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 351 aa, synthetic construct
  REGION 1..351: note = "Synthetic polypeptide: 3h56-269-IgG1.1f"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 351 aa, synthetic construct
  REGION 1..351: note = "Synthetic polypeptide: 3h56-269-IgG1.3f"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 351 aa, synthetic construct
  REGION 1..351: note = "Synthetic polypeptide: 3h56-269-IgG1-D265A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 218 aa, synthetic construct
  REGION 1..218: note = "Synthetic polypeptide: 1F4 heavy chain variable and CH1"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 81 - protein, 215 aa, synthetic construct
  REGION 1..215: note = "Synthetic polypeptide: 1F4 light chain variable and CL"
EIVLTQSPGTLSLSPGERATLSCRASQSISSSYLAWYQQKPGQAPRLLIYGASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 450 aa, synthetic construct
  REGION 1..450: note = "Synthetic polypeptide: 1F4-IgG1f heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 446 aa, synthetic construct
  REGION 1..446: note = "Synthetic polypeptide: 1F4-IgG4.1 heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 84 - protein, 449 aa, synthetic construct
  REGION 1..449: note = "Synthetic polypeptide: 1F4-IgG1.1f heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 449 aa, synthetic construct
  REGION 1..449: note = "Synthetic polypeptide: 1F4-IgG1.3f heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 448 aa, synthetic construct
  REGION 1..448: note = "Synthetic polypeptide: 1F4-D265A heavy chain"
EVQLLESGGGLVQPGGSLRLSCAASGFTFSIYAMSWVRQAGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIVEWESNGQPENNYKTTPPVLDSDGSFFLYSKLTVDKSRWQQGNVFSCSVMHEALHNHYTQKSLSLSPGK

SEQ ID NO: 87 - protein, 450 aa, synthetic construct
  REGION 1..450: note = "Synthetic polypeptide: 1F4-CT heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 351 aa, synthetic construct
  REGION 1..351: note = "Synthetic polypeptide: 3h56-269-CTf"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 351 aa, synthetic construct
  REGION 1..351: note = "Synthetic polypeptide: 3h56-269-IgG1a"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 351 aa, synthetic construct
  REGION 1..351: note = "Synthetic polypeptide: 3h56-269-IgG1a-C220S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 351 aa, synthetic construct
  REGION 1..351: note = "Synthetic polypeptide: 3h56-269-IgG1a-C220S, C226S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 351 aa, synthetic construct
  REGION 1..351: note = "Synthetic polypeptide: 3h56-269-IgG1a-C220S, C229S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 351 aa, synthetic construct
  REGION 1..351: note = "Synthetic polypeptide: 3h56-269-IgG1a-C220S, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 351 aa, synthetic construct
  REGION 1..351: note = "Synthetic polypeptide: 3h56-269-IgG1a-C220S, C226S, C229S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 351 aa, synthetic construct
  REGION 1..351: note = "Synthetic polypeptide: 3h56-269-IgG1a-C220S, C226S, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 351 aa, synthetic construct
  REGION 1..351: note = "Synthetic polypeptide: 3h56-269-IgG1a-C220S, C229S, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 351 aa, synthetic construct
  REGION 1..351: note = "Synthetic polypeptide: 3h56-269-IgG1a-C220S, C226A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 351 aa, synthetic construct
  REGION 1..351: note = "Synthetic polypeptide: 3h56-269-IgG1a-C220S, C229A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 351 aa, synthetic construct
  REGION 1..351: note = "Synthetic polypeptide: 3h56-269-IgG1a-C220S, C226A, C229A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 351 aa, synthetic construct
  REGION 1..351: note = "Synthetic polypeptide: 3h56-269-IgG1a-C220S, C226A, C229A, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPAPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 351 aa, synthetic construct
  REGION 1..351: note = "Synthetic polypeptide: 3h56-269-IgG1a-C220S, C226A, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 351 aa, synthetic construct
  REGION 1..351: note = "Synthetic polypeptide: 3h56-269-IgG1a-C220S, C229A, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPAPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 351 aa, synthetic construct
  REGION 1..351: note = "Synthetic polypeptide: 3h56-269-IgG1a-C220S, C226A, C229S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 351 aa, synthetic construct
  REGION 1..351: note = "Synthetic polypeptide: 3h56-269-IgG1a-C220S, C226S, C229A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 351 aa, synthetic construct
  REGION 1..351: note = "Synthetic polypeptide: 3h56-269-IgG1a-C220S, C226A, C229S, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 351 aa, synthetic construct
  REGION 1..351: note = "Synthetic polypeptide: 3h56-269-IgG1a-C220S, C226S, C229A, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPAPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 351 aa, synthetic construct
  REGION 1..351: note = "Synthetic polypeptide: 3h56-269-IgG1a-C220A, C226S, C229S, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSADKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 351 aa, synthetic construct
  REGION 1..351: note = "Synthetic polypeptide: h56-269-IgG1a-C220A, C226A, C229S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSADKTHTAPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 351 aa, synthetic construct
  REGION 1..351: note = "Synthetic polypeptide: 3h56-269-IgG1a-C220A, C226A, C229S, P238S"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSADKTHTAPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 351 aa, synthetic construct
  REGION 1..351: note = "Synthetic polypeptide: 3h56-269-IgG1a-C220S, P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 351 aa, synthetic construct
  REGION 1..351: note = "Synthetic polypeptide: 3h56-269-IgG1a-C220S, C226S, C229S, P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPSPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 351 aa, synthetic construct
  REGION 1..351: note = "Synthetic polypeptide: 3h56-269-IgG1a-C220S, C226A, C229A, P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPAPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 351 aa, synthetic construct
  REGION 1..351: note = "Synthetic polypeptide: 3h56-269-IgG1a-C220S, C226A, P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 351 aa, synthetic construct
  REGION 1..351: note = "Synthetic polypeptide: 3h56-269-IgG1a-C220S, C229A, P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPAPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 351 aa, synthetic construct
  REGION 1..351: note = "Synthetic polypeptide: 3h56-269-IgG1a-C220S, C226A, C229S, P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTAPPSPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 351 aa, synthetic construct
  REGION 1..351: note = "Synthetic polypeptide: 3h56-269-IgG1a-C220S, C229A, P238K"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTSPPAPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 351 aa, synthetic construct
  REGION 1..351: note = "Synthetic polypeptide: 3h56-269-IgG1a-C220S, L234A, L235A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 351 aa, synthetic construct
  REGION 1..351: note = "Synthetic polypeptide: 3h56-269-IgG1f-C220S, L234A, L235A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 351 aa, synthetic construct
  REGION 1..351: note = "Synthetic polypeptide: 3h56-269-IgG1a-C220S, N297A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 351 aa, synthetic construct
  REGION 1..351: note = "Synthetic polypeptide: 3h56-269-IgG1f-C220S, N297A"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 450 aa, synthetic construct
  REGION 1..450: note = "Synthetic polypeptide: 1F4-IgG1a-C226S heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 450 aa, synthetic construct
  REGION 1..450: note = "Synthetic polypeptide: 1F4- IgG1a-C229S heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 450 aa, synthetic construct
  REGION 1..450: note = "Synthetic polypeptide: 1F4- IgG1a-C226S-C229S heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 450 aa, synthetic construct
  REGION 1..450: note = "Synthetic polypeptide: 1F4- IgG1a-C226A heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 450 aa, synthetic construct
  REGION 1..450: note = "Synthetic polypeptide: 1F4- IgG1a-C229A heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 450 aa, synthetic construct
  REGION 1..450: note = "Synthetic polypeptide: 1F4- IgG1a-C226A-C229A heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 450 aa, synthetic construct
  REGION 1..450: note = "Synthetic polypeptide: 1F4- IgG1a-P238S heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 450 aa, synthetic construct
  REGION 1..450: note = "Synthetic polypeptide: 1F4- IgG1a-P238K heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 450 aa, synthetic construct
  REGION 1..450: note = "Synthetic polypeptide: 1F4-CTf heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, synthetic construct
  REGION 1..449: note = "Synthetic polypeptide: 1F4-N297A heavy chain"
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 351 aa, synthetic construct
  REGION 1..351: note = "Synthetic polypeptide: 3h56-269-IgG1f"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 351 aa, synthetic construct
  REGION 1..351: note = "Synthetic polypeptide: 3h56-269-IgG1-S267E"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 351 aa, synthetic construct
  REGION 1..351: note = "Synthetic polypeptide: 3h56-269-IgG1f-G237D, P238D, H268D, P271G, A330R"
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 231 aa, synthetic construct
  REGION 1..231: note = "Synthetic Peptide: IgG1a-P238K (-C-terminal Lys)
EPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 231 aa, synthetic construct
  REGION 1..231: note = Synthetic Peptide: IgG1f-P238K (-C-terminal Lys)
EPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 350 aa, synthetic construct
  REGION 1..350: note = Synthetic Peptide
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 350 aa, synthetic construct
  REGION 1..350: note = Synthetic Peptide
EVQLLESGGGLVQPGGSLRLSCAASGFTFRDYEMWWVRQAPGKGLERVSAINPQGTRTYYADSVKGRFTISRDNSKNTLYLQMNSLRAEDTAVYYCAKLPFRFSDRGQGTLVTVSSASTEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0042858 A1
(43) Pub. Date: Feb. 12, 2026

(54) Modified IgG1 Fc Domains and Anti-CD40 Domain Antibody Fusions Therewith

(71) Applicant: BRISTOL-MYERS SQUIBB COMPANY, Princeton, NJ (US)

(72) Inventors: Aaron Yamniuk, Vancouver (CA); Mary Struthers, Edison, NJ (US); Suzanne J. Suchard, Portland, OR (US)

(73) Assignee: BRISTOL-MYERS SQUIBB COMPANY, Princeton, NJ (US)

(21) Appl. No.: 19/313,261

(22) Filed: Aug. 28, 2025

Related U.S. Application Data
(63) Continuation of application No. 16/495,994.
(60) Provisional application No. 62/511,245, filed on May 25, 2017.

(51) Int. Cl. C07K 16/28

(52) U.S. Cl. CPC C07K 16/2878; C07K 2317/21; C07K 2317/41; C07K 2317/524; C07K 2317/526; C07K 2317/53; C07K 2317/569; C07K 2317/71; C07K 2317/72; C07K 2317/75; C07K 2317/92; C07K 2317/94

(57) ABSTRACT

Modified IgG1 Fc domains having reduced binding to Fc-gamma-receptors are provided. Further provided are fusion polypeptides comprising a modified IgG1 Fc domain. An antibody polypeptide that specifically binds a human CD40 is provided, wherein the antibody polypeptides are fusions of a domain antibody (dAb) comprising a single V_{H} domain and an Fc domain. The antibody polypeptides do not exhibit CD40 agonist activity, do not substantially activate immature dendritic cells, and have improved biophysical properties.

### CROSS REFERENCE TO RELATED APPLICATIONS

This application is a continuation of U.S. application Ser. No. 16/495,994, filed Sep. 20, 2019, which is the National Stage of International Application No. PCT/US2018/034330, filed May 24, 2018, and claims benefit of U.S. Provisional Application No. 62/511,245 filed on May 25, 2017.

### SEQUENCE LISTING

The instant application contains a Sequence Listing which has been submitted which has been submitted electronically in XML format and is hereby incorporated by reference in its entirety. Said XML copy, created on Aug. 26, 2025, is named 009616-00272-US.xml and is 207,334 bytes in size.

### TECHNICAL FIELD

Modified IgG1 Fc domains having reduced binding to Fc-gamma-receptors are provided. Antibody polypeptides comprising an anti-CD40 single variable domain and a modified Fc domain are provided. The antibody polypeptides bind CD40, do not exhibit CD40 agonist activity, do not activate immature dendritic cells, and have improved biophysical properties suitable for development as a therapeutic agent. Compositions comprising same, methods of use for treatment of diseases involving CD40 activity, and uses in the preparation of a medicament for treatment of disease involving CD40 activity are provided.

### BACKGROUND

CD40 is a co-stimulatory molecule belonging to the tumor necrosis factor (TNF) receptor superfamily that is present on antigen presenting cells (APC), including dendritic cells, B cells, and macrophages. APCs are activated when CD40 binds its ligand, CD154 (CD40L), on TH cells. CD40-mediated APC activation is involved in a variety of immune responses, including cytokine production, up-regulation of co-stimulatory molecules (such as CD86), and enhanced antigen presentation and B cell proliferation. CD40 can also be expressed by endothelial cells, smooth muscle cells, fibroblasts, and epithelial cells.

CD40 activation is also involved in a variety of undesired T cell responses related to autoimmunity, transplant rejection, or allergic responses, for example. One strategy for controlling undesirable T cell responses is to target CD40 with an antagonistic antibody. For example, monoclonal antibody HCD122 (Lucatumumab), formerly known as Chiron 1212, is currently in clinical trials for the treatment of certain CD40-mediated inflammatory diseases. See “Study of HCD122 (Lucatumumab) and Bendamustine Combination Therapy in CD40⁺ Rituximab-Refractory Follicular Lymphoma,” Clinical Trials Feeds, on the Internet at hypertext transfer protocol: clinicaltrialsfeeds.org/clinical-trials/show/NCT01275209 (last updated Jan. 11, 2011). Monoclonal antibodies, however, can display agonist activity. For example, the usefulness of the anti-CD40 antibody Chi220 is limited by its weak stimulatory potential. See Adams, et al., 2005, “Development of a chimeric anti-CD40 monoclonal antibody that synergizes with LEA29Y to prolong islet allograft survival,” J. Immunol. 174: 542-50.

There is an on-going need for therapeutics to modulate CD40 activation in the treatment and/or prevention of immune diseases.

### SUMMARY

Provided is a human IgG1 Fc domain polypeptide comprising a mutation at Kabat position 238 that reduces binding to Fc-gamma-receptors, wherein proline 238 (P238) is mutated to one of the residues selected from lysine, serine, alanine, arginine and tryptophan. The IgG1 Fc can comprise an amino acid sequence of SEQ ID NO: 65.

Provided is a human IgG1 Fc domain polypeptide comprising a lysine substituted at Kabat position 238. Exemplary amino acids sequences for the human IgG1 Fc domain polypeptide are:

EPKSCDKTHTCPPCPAPELLGGKSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPG (IgG1a-P238K (-C-terminal Lys);
SEQ ID NO: 134),
EPKSCDKTHTCPPCPAPELLGGKSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFITSDIAVEWESNGQPENNYKTTP
PVLDSDGSFELYSKLTVDKSRWQQGNVESCSVMHEALHNHYTQKS
LSLSPGK (IgGla-P238K; SEQ ID NO: 66),
EPKSCDKTHTCPPCPAPELLGGKSVFLEPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFYPSDIAVEWESNGQPENNYKTTP
PVLDSDGSFFLYSKLTVDKSRWQQGNVFSCSVMHEALHNHYTQKS
LSLSPG (IgG1f-P238K (-C-terminal Lys);
SEQ ID NO: 135),
and
EPKSCDKTHTCPPCPAPELLGGKSVFLEPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFITSDIAVEWESNGQPENNYKTTP
PVLDSDGSFFLYSKLTVDKSRWQQGNVESCSVMHEALHNHYTQKS
LSLSPGK (IgG1f-P238K; SEQ ID NO: 67).

Provided is a fusion polypeptide comprising: (A) a heterologous polypeptide; and (B) an Fc domain as described above.

Further provided is an antibody polypeptide comprising: (1) a single variable domain, said single variable domain comprising: (a) a CDR1 region comprising the amino acid sequence of SEQ ID NO: 1 or differing from the CDR1 region of SEQ ID NO: 1 by up to two amino acids, (b) a CDR2 region comprising the amino acid sequence of SEQ ID NO: 2 or differing from the CDR2 region of SEQ ID NO: 2 by up to three amino acids, and (c) a CDR3 region comprising the amino acid sequence of SEQ ID NO: 3 or differing from the CDR3 region of SEQ ID NO: 3 by up to six amino acids, and wherein said single variable domain binds CD40; and (2) an Fc domain that is a human IgG1 Fc domain polypeptide comprising a mutation at Kabat position 238 that reduces binding to Fc-gamma-receptors, wherein proline 238 (P238) is mutated to one of the residues selected from lysine, serine, alanine, arginine and tryptophan. The single variable domain of the antibody polypeptide described herein antagonizes at least one activity of CD40. The antibody polypeptide as described herein has increased stability, relative to a reference polypeptide that has the same single variable domain sequence and is fused to a wild-type IgG1 Fc domain. Provided is an antibody polypeptide comprising: (1) a single variable domain as described above, wherein the human IgG1 Fc domain has a lysine substituted at Kabat position 238. Exemplary amino acids sequences for the human IgG1 Fc domain polypeptide are:

EPKSCDKTHTCPPCPAPELLGGKSVFLFPPKPKDTLMISRTPEV
TCVVVDVSHEDPEVKFNWYVDGVEVHNAKTKPREEQYNSTYRVV
SVLTVLHQDWLNGKEYKCKVSNKALPAPIEKTISKAKGQPREPQ
VYTLPPSRDELTKNQVSLTCLVKGFYPSDIAVEWESNGQPENNY
KTTPPVLDSDGSFFLYSKLTVDKSRWQQGNVFSCSVMHEALHNH
YTQKSLSLSPG (IgG1a-P238K (-C-terminal Lys);
SEQ ID NO: 134),
EPKSCDKTHTCPPCPAPELLGGKSVFLEPPKPKDTLMISRTPEV
TCVVVDVSHEDPEVKFNWYVDGVEVHNAKTKPREEQYNSTYRVV
SVLTVLHQDWLNGKEYKCKVSNKALPAPIEKTISKAKGQPREPQ
VYTLPPSRDELTKNQVSLTCLVKGFYPSDIAVEWESNGQPENNY
KTTPPVLDSDGSFFLYSKLTVDKSRWQQGNVFSCSVMHEALHNH
YTQKSLSLSPGK (IgG1a-P238K; SEQ ID NO: 66),
EPKSCDKTHTCPPCPAPELLGGKSVFLEPPKPKDTLMISRTPEV
TCVVVDVSHEDPEVKFNWYVDGVEVHNAKTKPREEQYNSTYRVV
SVLTVLHQDWLNGKEYKCKVSNKALPAPIEKTISKAKGQPREPQ
VYTLPPSREEMTKNQVSLTCLVKGFYPSDIAVEWESNGQPENNY
KTTPPVLDSDGSFFLYSKLTVDKSRWQQGNVFSCSVMHEALHNH
YTQKSLSLSPG (IgG1 f-P238K (-C-terminal Lys);
SEQ ID NO: 135),
and
EPKSCDKTHTCPPCPAPELLGGKSVFLEPPKPKDTLMISRTPEV
TCVVVDVSHEDPEVKFNWYVDGVEVHNAKTKPREEQYNSTYRVV
SVLTVLHQDWLNGKEYKCKVSNKALPAPIEKTISKAKGQPREPQ
VYTLPPSREEMTKNQVSLTCLVKGFYPSDIAVEWESNGQPENNY
KTTPPVLDSDGSFFLYSKLTVDKSRWQQGNVFSCSVMHEALHNH
YTQKSLSLSPGK (IgG1f-P238K; SEQ ID NO: 67).

Further provided is an antibody polypeptide as described above, wherein (a) the CDR1 region consists of a sequence X1-Tyr-Glu-Y1-Trp (SEQ ID NO: 4), wherein X1 is Asp or Gly, and Y1 is Met or Leu; (b) the CDR2 region consists of a sequence Ala-Ile-Asn-Pro-X2-Gly-Y2-Z2-Thr-Tyr-Tyr-Ala-Asp-Ser-Val-A2-Gly (SEQ ID NO: 5), wherein X2 is Gln, Tyr, His, Trp, or Ala, Y2 is Thr, Asn, Gly, Ser, or Gln, Z2 is Arg, Leu, Tyr, His, or Phe, and A2 is Lys or Met; and (c) the CDR3 region consists of a sequence X3-Pro-Y3-Z3-A3-B3-C3 (SEQ ID NO: 6), wherein X3 is Leu, Pro, or Glu, Y3 is Phe, Gln, Thr, Met, or Tyr, Z3 is Arg, Tyr, Pro, Leu, Thr, Ile, Phe, Met, or Ser, A3 is Phe or Tyr, B3 is Ser, Gln, His, Asp, Lys, Glu, or Gly, and C3 is Asp, Tyr, Glu, or Ser.

Further provided is an antibody polypeptide as described above, wherein: (a) the CDR1 region consists of the amino acid sequence of SEQ ID NO: 1 (CDR1 of 3h-56-269), (b) the CDR2 region consists of the amino acid sequence of SEQ ID NO: 2 (CDR2 of 3h-56-269), and (c) the CDR3 region consists of the amino acid sequence of SEQ ID NO: 3 (CDR3 of 3h-56-269). Further provided is an antibody polypeptide as described above, wherein the amino acid sequence of the single variable domain is set forth in SEQ ID NO: 41 (=3h-56-269 sequence).

Provided is an antibody polypeptide comprising or consisting of the amino acid sequence:

(SEQ ID NO: 136)
EVQLLESGGGLVQPGGSLRLSCAASGFTERDYEMWWVRQAPGKGLERVSA
INPQGTRTYYADSVKGRFTISRDNSKNTLYLQMNSLRAEDTAVYYCAKLPF
RFSDRGQGTLVTVSSASTEPKSCDKTHTCPPCPAPELLGGKSVFLEPPKPK
DTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSD
GSFELYSKLTVDKSRWQQGNVESCSVMHEALHNHYTQKSLSLSPG,
or
(SEQ ID NO: 70)
EVQLLESGGGLVQPGGSLRLSCAASGFTFRDYEMWWVRQAPGKGLERVSA
INPQGTRTYYADSVKGRFTISRDNSKNTLYLQMNSLRAEDTAVYYCAKLPF
RFSDRGQGTLVTVSSASTEPKSCDKTHTCPPCPAPELLGGKSVFLFPPKPK
DTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.

Provided is an antibody polypeptide comprising or consisting of the amino acid sequence:

(SEQ ID NO: 137)
EVQLLESGGGLVQPGGSLRLSCAASGFTFRDYEMWWVRQAPGKGLER
VSAINPQGTRTYYADSVKGRFTISRDNSKNTLYLQMNSLRAEDTAVY
YCAKLPFRFSDRGQGTLVTVSSASTEPKSCDKTHTCPPCPAPELLGG
KSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAP
IEKTISKAKGQPREPQVYTLPPSREEMTKNQVSLTCLVKGFYPSDIA
VEWESNGQPENNYKTTPPVLDSDGSFFLYSKLTVDKSRWQQGNVESC
SVMHEALHNHYTQKSLSLSPG,
or
(SEQ ID NO: 71)
EVQLLESGGGLVQPGGSLRLSCAASGFTERDYEMWWVRQAPGKGLERV
SAINPQGTRTYYADSVKGRFTISRDNSKNTLYLQMNSLRAEDTAVYYC
AKLPFRFSDRGQGTLVTVSSASTEPKSCDKTHTCPPCPAPELLGGKSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESN
GQPENNYKTTPPVLDSDGSFELYSKLTVDKSRWQQGNVESCSVMHEAL
HNHYTQKSLSLSPGK.

Further provided is a nucleic acid encoding any of the human IgG1 Fc domain polypeptides, the fusion polypeptides, or the antibody polypeptides of the disclosure. An expression vector comprising the nucleic acid molecule is also provided. A cell transformed with the expression vector is provided.

A pharmaceutical composition comprising the antibody polypeptide described above, and a pharmaceutically acceptable carrier is provided.

A method of treating or preventing an immune disease in a subject comprising administering to the subject the antibody polypeptide described above is provided. The immune disease can be selected from the group consisting of Addison's disease, allergies, anaphylaxis, ankylosing spondylitis, asthma, atherosclerosis, atopic allergy, autoimmune diseases of the ear, autoimmune diseases of the eye, autoimmune hepatitis, autoimmune parotitis, bronchial asthma, coronary heart disease, Crohn's disease, diabetes, epididymitis, glomerulonephritis, Graves' disease, Guillain-Barre syndrome, Hashimoto's disease, hemolytic anemia, idiopathic thrombocytopenia purpura, inflammatory bowel disease, immune response to recombinant drug products (e.g., Factor VII in hemophiliacs), systemic lupus erythematosus, multiple sclerosis, myasthenia gravis, pemphigus, psoriasis, rheumatic fever, rheumatoid arthritis, sarcoidosis, scleroderma, Sjogren's syndrome, spondyloarthropathies, thyroiditis, transplant rejection, vasculitis, and ulcerative colitis.

### BRIEF DESCRIPTION OF THE FIGURES

FIGS. 1A and 1B depict amino acid sequences of representative antibody polypeptides useful of the disclosure. FIG. 1A depicts the amino acid sequence (SEQ ID NO: 70) of an antibody polypeptide fusion of a single variable domain antibody BMS3h-56-269 (SEQ ID NO: 41) and an Fc domain (IgG1a-P238K; SEQ ID NO: 66). The amino acid sequence of the Fc domain (SEQ ID NO: 66) is italicized; the underlined italicized residue 23 (corresponds to Kabat position 238) is a proline-to-lysine mutation. FIG. 1B depicts the amino acid sequence (SEQ ID NO: 71) of an antibody polypeptide fusion of a single variable domain antibody BMS3h-56-269 (SEQ ID NO: 41) and another Fc domain (IgG1f-P238K; SEQ ID NO: 67). The amino acid sequence of the Fc domain (SEQ ID NO: 67) is italicized. In both FIGS. 1A and 1B, the three complementarity-determining regions, CDR1 (SEQ ID NO: 1), CDR2 (SEQ ID NO: 2) and CDR3 (SEQ ID NO: 3) of the single variable domain are underlined. The amino acids of the four framework regions, FR1 (SEQ ID NO: 42), FR2 (SEQ ID NO: 44), FR3 (SEQ ID NO: 47) and FR4 (SEQ ID NO: 54) are not underlined. The linker amino acid sequence AST (SEQ ID NO: 57) is double-underlined.

FIGS. 2A-2E depict iDC activation data at various concentrations of dAb-Fc fusions for up to 9 iDC donors. FIGS. 2A-2D: Dose response of BMS-986090 (anti-CD40 dAb fused with IgG4 Fc), CD40L (a soluble CD40L trimer (via isoleucine zipper trimerization motif) and mAb 134-2141 (agonistic anti-CD40 antibody), on CD86 expression (FIG. 2A), ICAM-1 expression (FIG. 2B), IL-6 release (FIG. 2C) and TNF-alpha release (FIG. 2D). ChiL6-IgG4 (Control-L6): negative control. Unstim: iDCs alone (unstimulated). Concentrations in μg/ml are indicated. FIG. 2E depicts a comparison of BMS-986090 with 3h-59-269-aba (dAb-IgG1 fusion) at 100 μg/ml treated iDC from up to 9 donors.

FIGS. 3A-3D depict iDC activation is increased by CD32 mediated crosslinking/clustering of 3h-59-269-IgG4 as measured by CD86 expression (FIG. 3A), ICAM expression (FIG. 3B) and cytokine release (IL-6 in FIG. 3C and TNF-alpha in FIG. 3D). iDC treated with the indicated concentrations (in μg/ml) in solution or with cross-linking (‘x-link’) is indicated; cross-linking refers to the addition of CD32-expressing CHO cells. ChiL6-IgG4 serves as a negative control.

FIG. 4 depicts data from an iDC activation assay with anti-CD40 dAb with IgG4, IgG1.1f, IgG1.3f, and CT Fc tails. L6-IgG4 (ChiL6-IgG4) serves as a negative control; agonistic anti-CD40 mAb1234-2141 is a positive control. iDC treated with the indicated concentrations (μg/ml). Addition of CD32 expressing CHO cells to the iDC cultures (right panels) leads to a large increase in cytokine release and activation marker upregulation for all fusion proteins except for 3h-59-269-CT.

FIGS. 5A-5E depict DSC thermogram data for dAb-Fc molecules. FIG. 5A depicts data for 3h56-269-IgG4.1. FIG. 5B depicts data for 3h56-269-CT. FIG. 5C depicts data for 3h56-269-IgG1-D265A. FIG. 5D depicts data for 3h56-269-IgG1.1f. FIG. 5E depicts data for 3h56-269-IgG1.3f. In each panel, the thick line shows the thermogram data, and the thinner lines represent the simplest best fit.

FIGS. 6A-6F depict icIEF data for dAb-Fc molecules. FIG. 6A depicts data for 3h56-269-IgG4.1. FIG. 6B depicts data for 3h56-269-CT. FIG. 6C depicts data for 3h56-269-CT (produced from UCOE-CHO cells). FIG. 6D depicts data for 3h56-269-IgG1-D265A. FIG. 6E depicts data for 3h56-269-IgG1.1f. FIG. 6F depicts data for 3h56-269-IgG1.3f. The pI markers are indicated in panel A at pI 5.85 and pI 10.10.

FIG. 7 depicts SPR sensorgram data for the capture of 7 μg/ml hCD64-His with binding of four 1F4 antibodies at 1 μM.

FIG. 8 depicts icIEF data for thirteen 1F4 monoclonal antibodies having different Fc domains, with pI values labeled.

FIG. 9 depicts iDC activation data for 3h-59-269-IgG1-P238K and 3h-59-269-IgG1-N297A. L6-IgG4 (ChiL6-IgG4) serves as a negative control; BMS-986090 (3h-59-269-IgG4) is a positive control. iDC treated with the indicated concentrations (μg/ml) of antibody. Addition of CD32-expressing CHO cells to the iDC cultures (data on the left of each graph indicated by “+CD32 CHO”) leads to a large increase in cytokine release and activation marker upregulation for the positive control 3h-59-269-IgG4, but not with dAb fused to IgG1 Fc tails with a single mutation at position P238 or position N297.

FIG. 10 depicts iDC activation for anti-CD40 domain antibody-Fc fusion proteins with different Fc tails. L6-IgG4 (ChiL6-IgG4) serves as a negative control; agonistic mAb1234-2141 and BMS-986090 (3h-59-269-IgG4) are positive controls. iDCs are treated with the indicated concentrations (μg/ml) of antibody. Activation of iDCs is observed with all fusion proteins and is increased with the addition of CD32 expressing CHO (shown on the left of each graph indicated by “+CD32 CHO”), except for fusions containing a P238K or N297A mutation.

FIGS. 11A-11D depict DSC thermogram data for dAb-Fc molecules. FIG. 11A depicts data for 3h56-269-IgG1a-C220S,C226A,C229A,P238S. FIG. 11B depicts data for 3h56-269-IgG1a-C220S,C226A,C229A,P238K. FIG. 11C depicts data for 3h56-269-IgG1a-C220S,P238K. FIG. 11D depicts data for 3h56-269-IgG1f-C220S,N297A.

### DETAILED DESCRIPTION

In accordance with this detailed description, the following abbreviations and definitions apply. It must be noted that as used herein, the singular forms “a”, “an”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

As used here, the term “about” is understood by persons of ordinary skill in the art and will vary to some extent on the context in which it is used. Generally, about encompasses a range of values that are plus/minus 10% of a referenced value.

It is understood that any and all whole or partial integers between the ranges set forth here are included herein.

### Abbreviations Used Herein

| | |
|---|---|
| APC | antigen presenting cells |
| CD54 | also referred to as ICAM-1 |
| CDR | complementarity determining regions |
| C_{H} | constant heavy chain |
| C_{L} | constant light chain |
| CHO cell | Chinese hamster ovary cell |
| dAb | domain antibody |
| DSC | Differential scanning calorimetry |
| FcgR | Fc-gamma receptor (interchangeable with FcγR) |
| FR | Framework region |
| FSB | Fetal bovine serum |
| GM-CSF | granulocyte macrophage colony stimulating factor |
| iDC | immature dendritic cells |
| icIEF | Imaged capillary isoelectric focusing |
| IFN | interferon |
| IgG | Immunoglobulin G |
| IL-4 | Interleukin-4 |
| IL-6 | Interleukin-6 |
| mAb | monoclonal antibody |
| mg | milligram |
| ml or mL | milliliter |
| ng | nanogram |
| pI | isoelectric point |
| SPR | surface plasmon resonance |
| TNF | tumor necrosis factor |
| μg | microgram |
| V_{L} | variable light chain |
| V_{H} | variable heavy chain |

Further abbreviations and definitions are provided herein.

### 1. Fc Domain

The carboxy-terminal “half” of a heavy chain defines a constant region (Fc) primarily responsible for effector function. As used herein, the term “Fc domain” refers to the constant region antibody sequences comprising CH2 and CH3 constant domains as delimited according to Kabat et al., Sequences of Immunological Interest, 5th ed., U.S. Dept. Health & Human Services, Washington, D.C. (1991). The Fc domain disclosed herein is derived from a human IgG, and more specifically a human IgG1 Fc region. The human IgG1 Fc domain comprises a mutation at Kabat position 238. The mutation substitutes proline 238 (P238) with an amino acid selected from lysine (K), serine(S), alanine (A), arginine (R) and tryptophan (W); or selected from lysine and serine; or selected from lysine.

An exemplary consensus sequence for the IgG1 Fc domain is:
EPKSCDKTHTCPPCPAPELLGGXSVFLFPPKPKDTLMISRTPEVTCVVVDVSHEDPEV KFNWYVDGVEVHNAKTKPREEQYNSTYRVVSVLTVLHQDWLNGKEYKCKVSNKALPAPIEKTI SKAKGQPREPQVYTLPPSR (D/E) E (L/M) TKNQVSLTCLVKGFYPSDIAVEWESNGQPENNY KTTPPVLDSDGSFFLYSKLTVDKSRWQQGNVFSCSVMHEALHNHYTQKSLSLSPGZ (SEQ ID NO: 65), wherein X is K, S, A, R or W, and Z is K or absent. In this sequence, position 23 (the underlined X) corresponds to Kabat position 238.

Exemplary IgG Fc domain sequences are in Table 1. The mutated residue is underlined. SEQ ID NOS: 134 and 135 are additional exemplary IgG Fc domain sequences.

TABLE 1

| SEQ | |
|---|---|
| ID NO: | Amino acid sequence |
| 66 | EPKSCDKTHT CPPCPAPELL GGKSVFLFPP KPKDTLMISR |
| (IgG1a- | TPEVTCVVVD VSHEDPEVKF NWYVDGVEVH NAKTKPREEQ |
| P238K) | YNSTYRVVSV LTVLHQDWLN GKEYKCKVSN KALPAPIEKT |
| | ISKAKGQPRE PQVYTLPPSR DELTKNQVSL TCLVKGFYPS |
| | DIAVEWESNG QPENNYKTTP PVLDSDGSFF LYSKLTVDKS |
| | RWQQGNVFSC SVMHEALHNH YTQKSLSLSP GK |
| 67 | EPKSCDKTHT CPPCPAPELL GGKSVFLFPP KPKDTLMISR |
| (IgG1f- | TPEVTCVVVD VSHEDPEVKF NWYVDGVEVH NAKTKPREEQ |
| P238K) | YNSTYRVVSV LTVLHQDWLN GKEYKCKVSN KALPAPIEKT |
| | ISKAKGQPRE PQVYTLPPSR EEMTKNQVSL TCLVKGFYPS |
| | DIAVEWESNG QPENNYKTTP PVLDSDGSFF LYSKLTVDKS |
| | RWQQGNVFSC SVMHEALHNH YTQKSLSLSP GK |
| 68 | EPKSCDKTHT CPPCPAPELL GGSSVFLFPP KPKDTLMISR |
| (IgG1a- | TPEVTCVVVD VSHEDPEVKF NWYVDGVEVH NAKTKPREEQ |
| P238S) | YNSTYRVVSV LTVLHQDWLN GKEYKCKVSN KALPAPIEKT |
| | ISKAKGQPRE PQVYTLPPSR DELTKNQVSL TCLVKGFYPS |
| | DIAVEWESNG QPENNYKTTP PVLDSDGSFF LYSKLTVDKS |
| | RWQQGNVFSC SVMHEALHNH YTQKSLSLSP GK |
| 69 | EPKSCDKTHT CPPCPAPELL GGSSVFLFPP KPKDTLMISR |
| (Ig1f- | TPEVTCVVVD VSHEDPEVKF NWYVDGVEVH NAKTKPREEQ |
| P238S) | YNSTYRVVSV LTVLHQDWLN GKEYKCKVSN KALPAPIEKT |
| | ISKAKGQPRE PQVYTLPPSR EEMTKNQVSL TCLVKGFYPS |
| | DIAVEWESNG QPENNYKTTP PVLDSDGSFF LYSKLTVDKS |
| | RWQQGNVFSC SVMHEALHNH YTQKSLSLSP GK |

While human IgG heavy chain genes encode a C-terminal lysine, the lysine is often absent from endogenous antibodies as a result of cleavage in blood circulation. Antibodies having IgG heavy chains including a C-terminal lysine, when expressed in mammalian cell cultures, may also have variable levels of C-terminal lysine present (Cai et al, 2011, Biotechnol Bioeng. 108 (2): 404-12). Accordingly, the C-terminal lysine of any IgG heavy chain Fc domain disclosed herein may be omitted. See, for instance, SEQ ID NOs: 66 and 134, and SEQ ID NOS: 67 and 135. Similarly, the lysine at the C-terminal of SEQ ID NO: 68 and SEQ ID NO: 69 may optionally be absent.

The mutated IgG1 Fc domain exhibits reduced binding to Fc gamma receptors. Advantageously, the reduced binding to Fc gamma receptors reduces or precludes iDC activation as measured by at least one of: 1) release of cytokine IL-6 and/or TNF-alpha; and 2) upregulation of cell surface expression of CD86 and/or CD54. The reduced binding to Fc gamma receptors is also believed to reduce or preclude clustering/crosslinking of FcgRs on immature dendritic cells. Additionally, the mutated IgG1 Fc domain can contribute to thermal stability and homogeneity of antibody polypeptides comprising the mutated IgG1 Fc domain.

### 2. Antibody Polypeptides Comprising Mutated IgG1 Fc Domain

The present disclosure includes a fusion polypeptide comprising a mutated IgG1 Fc domain.

### 2.1. Heterologous Polypeptide

The present disclosure includes a fusion polypeptide of a heterologous polypeptide and a mutated IgG1 Fc domain of the disclosure. The heterologous polypeptide can comprise or consist of a heavy chain variable domain. The carboxyl terminus of the heavy chain variable domain may be linked or fused to the amino terminus of the Fc domain. Alternatively, the carboxyl terminus of the heavy chain variable domain may be linked or fused to the amino terminus of a linker amino acid sequence, which itself is fused to the amino terminus of the Fc domain. Alternatively, the carboxyl terminus of the heavy chain variable domain may be linked or fused to the amino terminus of a CH1 domain, which itself is fused to the Fc domain. The fusion polypeptide may comprise the hinge region between the CHI and CH2 domains in whole or in part. Optionally, an amino acid linker sequence is present between the heavy chain variable domain and the Fc domain.

### 2.2. Domain Antibody

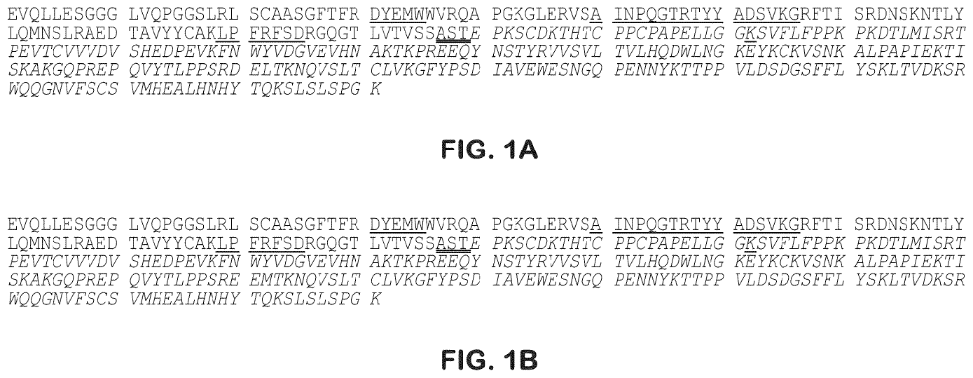

The present disclosure further includes a single variable domain (a domain antibody) is fused to an Fc domain. A “domain antibody” (dAb) comprises a single variable domain (VL or VH) domain that is capable of specifically and monovalently binding an antigen, such as CD40. The carboxyl terminus of the single variable domain may be linked or fused to the amino terminus of the Fc CH2 domain. Alternatively, the carboxyl terminus of the single variable domain may be linked or fused to the amino terminus of a linker amino acid sequence, which itself is fused to the amino terminus of an Fc domain. Alternatively, the carboxyl terminus of the variable domain may be linked or fused to the amino terminus of a CH1 domain, which itself is fused to the Fc CH2 domain. The protein may comprise the hinge region between the CH1 and CH2 domains in whole or in part. Optionally, an amino acid linker sequence is present between the single variable domain and the Fc domain. Also provided are antibody polypeptides that are fusion polypeptides comprising an anti-human CD40 domain antibody and a modified human Fc domain. Optionally, the antibody polypeptides further comprise an amino acid linker intervening between the domain antibody and the Fc domain. Exemplary antibody polypeptides are depicted in FIGS. 1A and 1B.

### 2.2.1. Anti-CD40 Domain Antibody

The antibody polypeptides of the disclosure comprise a domain antibody that specifically binds human CD40 and does not exhibit CD40 agonist activity. A “domain antibody” (dAb) comprises a single variable domain (VL or VH) that is capable of specifically and monovalently binding an antigen, such as CD40. The domain antibodies contain a “VH domain” and are human. Bivalent anti-CD40 antibodies are believed to exhibit agonist activity because of their ability to cross-link bound CD40 molecules on the cell surface. While not limited by any particular theory, it is believed that monovalent dAbs do not activate CD40, because the dAbs do not cross-link CD40.

CD40 is also known as B-cell surface antigen CD40, Bp50, CD40L receptor, CDw40, CDW40, MGC9013, p50, TNFRSF5, and Tumor necrosis factor receptor superfamily member 5. “Human CD40” refers to the CD40 comprising the following amino acid sequence:

(SEQ ID NO: 64)
MVRLPLQCVL WGCLLTAVHP EPPTACREKQ YLINSQCCSL
CQPGQKLVSD CTEFTETECL PCGESEFLDT WNRETHCHQH
KYCDPNLGLR VQQKGTSETD TICTCEEGWH CTSEACESCV
LHRSCSPGFG VKQIATGVSD TICEPCPVGF FSNVSSAFEK
CHPWTSCETK DLVVQQAGTN KTDVVCGPQD RLRALVVIPI
IFGILFAILL VLVFIKKVAK KPTNKAPHPK QEPQEINFPD
DLPGSNTAAP VQETLHGCQP VTQEDGKESR ISVQERQ.

As used herein, the term “variable domain” refers to immunoglobulin variable domains defined by Kabat et al., Sequences of Immunological Interest, 5th ed., U.S. Dept. Health & Human Services, Washington, D.C. (1991). The numbering and positioning of CDR amino acid residues within the variable domains is in accordance with the well-known Kabat numbering convention. For example, the Kabat numbering for BMS3h-56-269 (SEQ ID NO: 41) is compared in TABLE 2 to the same sequence wherein the amino acids are numbered sequentially. In the Kabat numbering, BMS3h-56-269 has insertion residues 52A, 82A, 82B, 82C, and is missing residue 100.

```
TABLE 2
                                                                                      CDR1
Kabat 10 20 30 40 5̅0
                                  — | — | — | — | — |
Sequential 10 20 30 40 50
                                  — | — | — | — | — |
BMS3h-56-269 EVQLLESGGGLVQPGGSLRLSCAASGFTFRDYEMWWVRQAPGKGLERVSA
                                              CDR2
Kabat 60 70 80 90
                                        A — | — | — | ABC — | —
Sequential 60 70 80 90 100
                                            — | — | — | — | — |
BMS3h-56-269 INPQGTRTYYADSVKGRFTISRDNSKNTLYLQMNSLRAEDTAVYYCAKLP
                                      CDR3
Kabat 101 110 113
                                            | — | |
Sequential 110 110 116
                                              — | —|
BMS3h-56-269 FRFSDRGQGTLVTVSS (SEQ ID NO: 41)
```

The term “human,” when applied to antibody polypeptides, means that the antibody polypeptide has a sequence, e.g., FR and/or CH domains, derived from a human immunoglobulin. A sequence is “derived from” a human immunoglobulin coding sequence when the sequence is either: (a) isolated from a human individual or from a cell or cell line from a human individual; (b) isolated from a library of cloned human antibody gene sequences or of human antibody variable domain sequences; or (c) diversified by mutation and selection from one or more of the polypeptides above. An “isolated” compound as used herein means that the compound is removed from at least one component with which the compound is naturally associated with in nature.

As used herein, “specific binding” refers to the binding of an antigen by an antibody polypeptide with a dissociation constant (Kd) of about 1 □M or lower as measured, for example, by surface plasmon resonance. Suitable assay systems include the BIAcore™ surface plasmon resonance (SPR) system and BIAcore™ kinetic evaluation software (e.g., version 2.1).

Binding of the present antibody polypeptides to CD40 antagonizes CD40 activity. “CD40 activities” include, but are not limited to, T cell activation (e.g., induction of T cell proliferation or cytokine secretion), macrophage activation (e.g., the induction of reactive oxygen species and nitric oxide in the macrophage), and B cell activation (e.g., B cell proliferation, antibody isotype switching, or differentiation to plasma cells). CD40 activities can be mediated by interaction with other molecules. “CD40 activities” include the functional interaction between CD40 and the following molecules, which are identified by their Uniprot Accession Number is parentheses:

| | |
|---|---|
| CALR | (P27797); |
| ERP44 | (Q9BS26); |
| FBL | (P22087); |
| POLR2H | (P52434); |
| RFC5 | (P40937); |
| SGK1 | (O00141); |
| SLC30A7 | (Q8NEW0); |
| SLC39A7 | (Q92504); |
| TRAF2 | (Q5T1L5); |
| TRAF3 | (Q13114); |
| TRAF6 | (Q9Y4K3); |
| TXN | (Q5T937); |
| UGGT1 | (Q9NYU2); and |
| USP15 | (Q9Y4E8). |

For example, a CD40 “activity” includes an interaction with TRAF2. CD40/TRAF2 interaction activates NF-κB and JNK. See Davies et al., Mol. Cell Biol. 25:9806-19 (2005). This CD40 activity thus can be determined by CD40-dependent cellular NF-κB and JNK activation, relative to a reference. As used herein, the terms “activate,” “activates,” and “activated” refer to an increase in a given measurable CD40 activity by at least 10% relative to a reference, for example, at least 10%, 25%, 50%, 75%, 80%, 90%, or even 100%, or more. A CD40 activity is “antagonized” if the activity is reduced by at least 10%, and in an exemplary embodiment, at least about 20%, 30%, 40%, 50%, 60%, 70%, 80%, 90%, 95%, 97%, 98%, 99%, or even 100% (i.e., no detectable activity), relative to the absence of the antagonist. For example, an antibody polypeptide may antagonize some or all CD40 activity, while not activating CD40. In one embodiment, the antibody polypeptide does not activate B cell proliferation. In another embodiment, the antibody polypeptide does not activate cytokine secretion by T cells, where the cytokine is at least one cytokine selected from the group consisting of IL-2, IL-6, IL-10, IL-13, TNF-α, and IFN-γ.

Antibody polypeptides of the present disclosure can be administered to human patients while largely avoiding the anti-antibody immune response often provoked by the administration of antibodies from other species, e.g., mouse. For example, murine antibodies can be “humanized” by grafting murine CDRs onto a human variable domain FR, according to procedures well known in the art. Human antibodies as disclosed herein, however, can be produced without the need for genetic manipulation of a murine antibody sequence.

The anti-CD40 domain antibodies useful in the present disclosure comprise three complementarity-determining regions (CDRs) and four framework regions (FRs), arranged from amino-terminus to carboxy-terminus in the following order: FR1, CDR1, FR2, CDR2, FR3, CDR3, FR4. The three CDRs contain most of the residues that form specific interactions with the antigen and are primarily responsible for antigen recognition.

A genus of single variable domain antibody polypeptides that specifically bind a single CD40 epitope is described in U.S. Publication No. 2014/0099317, published Apr. 10, 2014, entitled “ANTIBODY POLYPEPTIDES THAT ANTAGONIZE CD40,” which is incorporated herein by reference in its entirety. The antibody polypeptides were characterized structurally and functionally, and that data is also described in U.S. Publication No. 2014/0099317, published Apr. 10, 2014. BMS3h-56-269 is an exemplary single variable domain antibody polypeptide that specifically binds to, but does not agonize, human CD40, as disclosed in U.S. Publication No. 2014/0099317.

The CDRs contain most of the residues that form specific interactions with the antigen. The single variable domain of an antibody polypeptide of the present disclosure comprises CDR1, CDR2, and CDR3 regions that have the same amino acid sequence as the CDR1, CDR2, and CDR3 regions of BMS3h-56-269 (SEQ ID NO: 41) or that each differ from the CDR1, CDR2, and CDR3 regions by one, two, three, four, five, or six amino acids.

The amino acid sequence of BMS3h-56-269 (SEQ ID NO: 41) is shown below.

EVQLLESGGGLVQPGGSLRLSCAASGFTFRDYEMWWVRQAPGKGLERVS
AINPQGTRTYYADSVKGRFTISRDNSKNTLYLQMNSLRAEDTAVYYCAK
LPFRFSDRGQGTLVTVSS

The amino acids of the three complementarity-determining regions are underlined. The amino acid sequence of CDR1 is DYEMW (SEQ ID NO: 1). The amino acid sequence of CDR2 is AINPQGTRTYYADSVKG (SEQ ID NO: 2), and the amino acid sequence of CDR3 is LPFRFSD (SEQ ID NO: 3). An exemplary nucleic acid sequence encoding the amino acid sequence of BMS3h-56-269 is:

(SEQ ID NO: 72)
gaggtgcagc tgttggagtc tgggggaggc ttggtacagc
ctggggggtc cctgcgtctc tcctgtgcag cctccggatt
cacctttcgg gattatgaga tgtggtgggt ccgccaggct
ccagggaagg gtctagagcg ggtctcagct attaatccgc
agggtacgcg tacatactac gcagactccg tgaagggccg
gttcaccatc tcccgcgaca attccaagaa cacgctgtat
ctgcaaatga acagcctgcg tgccgaggat accgcggtat
attactgtgc gaaacttccg tttaggtttt ccgaccgggg
tcagggaacc ctggtcaccg tctcgagc.

The variable domain of an antibody polypeptide provided by the disclosure comprises CDR1, CDR2, and CDR3 regions that have the same amino acid sequence as the CDR1, CDR2, and CDR3 regions of BMS3h-56-269 (SEQ ID NOs: 1-3, respectively) or that each differ from the CDR1, CDR2, and CDR3 regions by one, two, three, four, five, or six amino acids. The CDR1 region may vary by up to two amino acids from SEQ ID NO: 1. The CDR2 region may vary by up to three amino acids from SEQ ID NO: 2. The CDR3 region may vary by up to six amino acids from SEQ ID NO: 3. Thus, the variable domain of an antibody polypeptide can comprise: (a) a CDR1 region comprising the amino acid sequence of SEQ ID NO: 1 or differing from the CDR1 region of SEQ ID NO: 1 by up to two amino acids, (b) a CDR2 region comprising the amino acid sequence of SEQ ID NO: 2 or differing from the CDR2 region of SEQ ID NO: 2 by up to three amino acids, and (c) a CDR3 region comprising the amino acid sequence of SEQ ID NO: 3 or differing from the CDR3 region of SEQ ID NO: 3 by up to six amino acids, and wherein said single variable domain binds CD40. The variable domain of an antibody polypeptide can comprise: (a) a CDR1 region consisting of the amino acid sequence of SEQ ID NO: 1 or differing from the CDR1 region of SEQ ID NO: 1 by up to two amino acids, (b) a CDR2 region consisting of the amino acid sequence of SEQ ID NO: 2 or differing from the CDR2 region of SEQ ID NO: 2 by up to three amino acids, and (c) a CDR3 region consisting of the amino acid sequence of SEQ ID NO: 3 or differing from the CDR3 region of SEQ ID NO: 3 by up to six amino acids, and wherein said single variable domain binds CD40.

Exemplary antibody polypeptides are described in Section 2.5. Further exemplary antibodies are described here.

The variable domain of an antibody polypeptide disclosed herein can comprise (a) a CDR1 region that consists of a sequence X1-Tyr-Glu-Y1-Trp (SEQ ID NO: 4), wherein X1 is Asp or Gly, and Y1 is Met or Leu; (b) a CDR2 region that consists of a sequence Ala-Ile-Asn-Pro-X2-Gly-Y2-Z2-Thr-Tyr-Tyr-Ala-Asp-Ser-Val-A2-Gly (SEQ ID NO: 5), wherein X2 is Gln, Tyr, His, Trp, or Ala, Y2 is Thr, Asn, Gly, Ser, or Gln, Z2 is Arg, Leu, Tyr, His, or Phe, and A2 is Lys or Met; and (c) a CDR3 region that consists of a sequence X3-Pro-Y3-Z3-A3-B3-C3 (SEQ ID NO: 6), wherein X3 is Leu, Pro, or Glu, Y3 is Phe, Gln, Thr, Met, or Tyr, Z3 is Arg, Tyr, Pro, Leu, Thr, Ile, Phe, Met, or Ser, A3 is Phe or Tyr, B3 is Ser, Gln, His, Asp, Lys, Glu, or Gly, and C3 is Asp, Tyr, Glu, or Ser.

The variable domain of an antibody polypeptide disclosed herein can comprise (a) a CDR1 region that consists of a sequence X1-Tyr-Glu-Y1-Trp (SEQ ID NO: 4), wherein X1 is Asp, and Y1 is Met; (b) a CDR2 region that consists of a sequence Ala-Ile-Asn-Pro-X2-Gly-Y2-Z2-Thr-Tyr-Tyr-Ala-Asp-Ser-Val-A2-Gly (SEQ ID NO: 5), wherein X2 is Gln, Tyr, His, Trp, or Ala, Y2 is Thr, Asn, Gly, Ser, or Gln, Z2 is Arg, Leu, Tyr, His, or Phe, and A2 is Lys; and (c) a CDR3 region that consists of a sequence X3-Pro-Y3-Z3-A3-B3-C3 (SEQ ID NO: 6), wherein X3 is Leu, Y3 is Phe, Gln, Thr, or Met, Z3 is Arg, Tyr, Leu, Thr, or Phe, A3 is Phe, B3 is Ser, Gln, His, Asp, or Glu, and C3 is Asp or Glu.

| CDR1 | SEQ ID NO | CDR2 | SEQ ID NO | CDR3 | SEQ ID NO |
|---|---|---|---|---|---|
| DYEMW | 1 | AINPQGTRTYYADSVKG | 2 | LPFRFSD | 3 |
| DYEMW | 1 | AINPQGTRTYYADSVKG | 2 | LPFTFED | 7 |
| DYEMW | 1 | AINPQGTRTYYADSVKG | 2 | LPFTFDD | 8 |
| DYEMW | 1 | AINPQGTRTYYADSVKG | 2 | LPTYFSD | 9 |
| DYEMW | 1 | AINPQGTRTYYADSVKG | 2 | LPFYFSE | 10 |
| DYEMW | 1 | AINPQGTRTYYADSVKG | 2 | LPFPFSE | 11 |
| DYEMW | 1 | AINPQGTRTYYADSVKG | 2 | PPFIFGD | 12 |
| DYEMW | 1 | AINPQGTRTYYADSVKG | 2 | LPFIFEY | 13 |
| DYEMW | 1 | AINPQGTRTYYADSVKG | 2 | LPFYFQE | 14 |
| DYEMW | 1 | AINPQGTRTYYADSVKG | 2 | LPFFFEE | 15 |
| DYEMW | 1 | AINPQGTRTYYADSVKG | 2 | LPTYFKD | 16 |
| DYEMW | 1 | AINPQGTRTYYADSVKG | 2 | LPMFFED | 17 |
| DYEMW | 1 | AINPQGTRTYYADSVKG | 2 | LPFYFHE | 18 |
| DYEMW | 1 | AINPQGTRTYYADSVKG | 2 | LPFFFQE | 19 |
| DYEMW | 1 | AINPQGTRTYYADSVKG | 2 | LPQLFHD | 20 |
| DYEMW | 1 | AINPQGTRTYYADSVKG | 2 | LPQLFQD | 21 |
| DYEMW | 1 | AINPQGTRTYYADSVKG | 2 | LPFFFHE | 22 |
| DYEMW | 1 | AINPQGTRTYYADSVKG | 2 | LPTLFQD | 23 |
| DYEMW | 1 | AINPQGTRTYYADSVMG | 27 | LPFTFED | 7 |
| DYEMW | 1 | AINPQGTRTYYADSVMG | 27 | LPFTFDD | 8 |
| DYEMW | 1 | AINPQGTRTYYADSVMG | 27 | LPFMFDD | 24 |
| DYEMW | 1 | AINPQGTRTYYADSVMG | 27 | EPYSFDS | 25 |
| DYEMW | 1 | AINPQGTRTYYADSVMG | 27 | EPYSYDY | 26 |
| DYEMW | 1 | AINPWGTLTYYADSVKG | 28 | LPFTFED | 7 |
| DYEMW | 1 | AINPHGSLTYYADSVKG | 29 | LPFTFDD | 8 |
| DYEMW | 1 | AINPWGSLTYYADSVKG | 30 | LPFTFED | 7 |
| DYEMW | 1 | AINPYGGLTYYADSVKG | 31 | LPFTFDD | 8 |
| DYEMW | 1 | AINPWGQLTYYADSVKG | 32 | LPFTFED | 7 |
| DYEMW | 1 | AINPWGSHTYYADSVKG | 33 | LPFTFDD | 8 |
| DYEMW | 1 | AINPWGSLTYYADSVKG | 34 | LPFTFDD | 8 |
| DYEMW | 1 | AINPWGGFTYYADSVKG | 35 | LPFTFED | 7 |
| DYEMW | 1 | AINPWGGLTYYADSVKG | 36 | LPFTFDD | 8 |
| DYEMW | 1 | AINPAGGYTYYADSVKG | 37 | LPFTFED | 7 |
| DYEMW | 1 | AINPYGNLTYYADSVKG | 38 | LPFTFDD | 8 |
| GYEMW | 39 | AINPQGTRTYYADSVMG | 27 | LPFTFDD | 8 |
| GYEMW | 39 | AINPQGTRTYYADSVMG | 27 | LPFMFDD | 24 |
| GYELW | 40 | AINPQGTRTYYADSVMG | 27 | LPFTFDD | 8 |
| GYELW | 40 | AINPQGTRTYYADSVMG | 27 | LPFMFDD | 24 |

The variable domain of an antibody polypeptide disclosed herein can comprise (a) a CDR1 region that consists of the amino acid sequence of SEQ ID NO: 1; (b) a CDR2 region that consists of the amino acid sequence of SEQ ID NO: 2; and (c) a CDR3 region that consists of an amino acid sequence selected from the group consisting of: SEQ ID NO: 3, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO; 16, SEQ ID NO: 17, SEQ ID NO: 18, SEQ ID NO: 19, SEQ ID NO: 20, SEQ ID NO: 21, SEQ ID NO: 22, and SEQ ID NO: 23.

The variable domain of an antibody polypeptide disclosed herein can comprise (a) a CDR1 region that consists of the amino acid sequence of SEQ ID NO: 1; (b) a CDR2 region that consists of the amino acid sequence of SEQ ID NO: 27; and (c) a CDR3 region that consists of an amino acid sequence selected from the group consisting of: SEQ ID NO: 7, SEQ ID NO: 8, SEQ ID NO: 24, SEQ ID NO: 25, and SEQ ID NO: 26.

The variable domain of an antibody polypeptide disclosed herein can comprise (a) a CDR I region that consists of the amino acid sequence of SEQ ID NO: 1; (b) a CDR2 region that consists of an amino acid sequence selected from the group consisting of: SEQ ID NO: 28, SEQ ID NO: 30, SEQ ID NO: 32, SEQ ID NO: 35, and SEQ ID NO: 37; and (c) a CDR3 region that consists of the amino acid sequence of SEQ ID NO: 7.

The variable domain of an antibody polypeptide disclosed herein can comprise (a) a CDR1 region that consists of the amino acid sequence of SEQ ID NO: 1; (b) a CDR2 region that consists of an amino acid sequence selected from the group consisting of: SEQ ID NO: 29, SEQ ID NO: 31, SEQ ID NO: 33, SEQ ID NO: 34, SEQ ID NO: 36, and SEQ ID NO: 38; and (c) a CDR3 region that consists of the amino acid sequence of SEQ ID NO: 8.

The variable domain of an antibody polypeptide disclosed herein can comprise (a) a CDR I region that consists an amino acid sequence selected from the group consisting of: SEQ ID NO: 39 and SEQ ID NO: 40; (b) the CDR2 region consists of the amino acid sequence of SEQ ID NO: 27; and (c) the CDR3 region consists of an amino acid sequence selected from the group consisting of: SEQ ID NO: 8 and SEQ ID NO: 24.

The variable domain of an antibody polypeptide disclosed herein can comprise (a) a CDR1 region that consists of the amino acid sequence of SEQ ID NO: 1; (b) the CDR2 region consists of the amino acid sequence of SEQ ID NO: 2, and (c) the CDR3 region consists of the amino acid sequence of SEQ ID NO: 3. The variable domain of an antibody polypeptide disclosed herein can comprise or consist of the amino acid sequence of SEQ ID NO: 41 (3h-56-269 sequence).

The variable domain of an antibody polypeptide disclosed herein can comprise a CDR1 region, a CDR2 region, and a CDR3 region, wherein the amino acid sequence of the CDR1 region, the amino acid sequence of the CDR2 region, and the amino acid sequence of the CDR3 region are selected from the group consisting of:
(1) SEQ ID NO: 1, SEQ ID NO: 2, and SEQ ID NO: 3, respectively;
(2) SEQ ID NO: 1, SEQ ID NO: 2, and SEQ ID NO: 7, respectively;
(3) SEQ ID NO: 1, SEQ ID NO: 2, and SEQ ID NO: 8, respectively;
(4) SEQ ID NO: 1, SEQ ID NO: 2, and SEQ ID NO: 9, respectively;
(5) SEQ ID NO: 1, SEQ ID NO: 2, and SEQ ID NO: 10, respectively;
(6) SEQ ID NO: 1, SEQ ID NO: 2, and SEQ ID NO: 11, respectively;
(7) SEQ ID NO: 1, SEQ ID NO: 2, and SEQ ID NO: 12, respectively;
(8) SEQ ID NO: 1, SEQ ID NO: 2, and SEQ ID NO: 13, respectively;
(9) SEQ ID NO: 1, SEQ ID NO: 2, and SEQ ID NO: 14, respectively;
(10) SEQ ID NO: 1, SEQ ID NO: 2, and SEQ ID NO: 15, respectively;
(11) SEQ ID NO: 1, SEQ ID NO: 2, and SEQ ID NO: 16, respectively;
(12) SEQ ID NO: 1, SEQ ID NO: 2, and SEQ ID NO: 17, respectively;
(13) SEQ ID NO: 1, SEQ ID NO: 2, and SEQ ID NO: 18, respectively;
(14) SEQ ID NO: 1, SEQ ID NO: 2, and SEQ ID NO: 19, respectively;
(15) SEQ ID NO: 1, SEQ ID NO: 2, and SEQ ID NO: 20, respectively;
(16) SEQ ID NO: 1, SEQ ID NO: 2, and SEQ ID NO: 21, respectively;
(17) SEQ ID NO: 1, SEQ ID NO: 2, and SEQ ID NO: 22, respectively;
(18) SEQ ID NO: 1, SEQ ID NO: 2, and SEQ ID NO: 23, respectively;
(19) SEQ ID NO: 1, SEQ ID NO: 27 and SEQ ID NO: 7, respectively;
(20) SEQ ID NO: 1, SEQ ID NO: 27 and SEQ ID NO: 8, respectively;
(21) SEQ ID NO: 1, SEQ ID NO: 27 and SEQ ID NO: 24, respectively;
(22) SEQ ID NO: 1, SEQ ID NO: 27 and SEQ ID NO: 25, respectively;
(23) SEQ ID NO: 1, SEQ ID NO: 27 and SEQ ID NO: 26, respectively;
(24) SEQ ID NO: 1, SEQ ID NO: 28 and SEQ ID NO: 7, respectively;
(25) SEQ ID NO: 1, SEQ ID NO: 29 and SEQ ID NO: 8, respectively;
(26) SEQ ID NO: 1, SEQ ID NO: 30 and SEQ ID NO: 7, respectively;
(27) SEQ ID NO: 1, SEQ ID NO: 31 and SEQ ID NO: 8, respectively;
(28) SEQ ID NO: 1, SEQ ID NO: 32 and SEQ ID NO: 7, respectively;
(29) SEQ ID NO: 1, SEQ ID NO: 33 and SEQ ID NO: 8, respectively;
(30) SEQ ID NO: 1, SEQ ID NO: 34 and SEQ ID NO: 8, respectively;
(31) SEQ ID NO: 1, SEQ ID NO: 35 and SEQ ID NO: 7, respectively;
(32) SEQ ID NO: 1, SEQ ID NO: 36 and SEQ ID NO: 8, respectively;
(33) SEQ ID NO: 1, SEQ ID NO: 37 and SEQ ID NO: 7, respectively;
(34) SEQ ID NO: 1, SEQ ID NO: 38 and SEQ ID NO: 8, respectively;
(35) SEQ ID NO: 39, SEQ ID NO: 27 and SEQ ID NO: 8, respectively;
(36) SEQ ID NO: 39, SEQ ID NO: 27 and SEQ ID NO: 24, respectively;
(37) SEQ ID NO: 40, SEQ ID NO: 27 and SEQ ID NO: 8, respectively; and
(38) SEQ ID NO: 40, SEQ ID NO: 27 and SEQ ID NO: 24, respectively.

A variable domain in the antibody polypeptide may differ from the variable domain of BMS3h-56-269 by up to 10 amino acids or any integral value between, where the variant variable domain specifically binds CD40. Alternatively, the variant variable domain may have at least 90% sequence identity (e.g., at least 92%, 95%, or 98% sequence identity) relative to the sequence of BMS3h-56-269. Non-identical amino acid residues or amino acids that differ between two sequences may represent amino acid substitutions, additions, or deletions. Residues that differ between two sequences appear as non-identical positions, when the two sequences are aligned by any appropriate amino acid sequence alignment algorithm, such as BLAST.

Variable domains may comprise one or more framework regions (FR) with the same amino acid sequence as a corresponding framework region encoded by a human germline antibody gene segment. For example, a domain antibody may comprise the VH germline gene segments DP47, DP45, or DP38, the VK germline gene segment DPK9, the JH segment JH4b, or the Jκ segment Jκ1.

Exemplary framework regions include the framework regions from 3h-56-269: FR1=EVQLLESGGGLVQPGGSLRLSCAASGFTFR (amino acids 1-30 of 3h-56-269); FR2=WVRQAPGKGLERVS (amino acids 36-49 of 3h-56-269); FR3=RFTISRDNSKNTLYLQMNSLRAEDTAVYYCAK (amino acids 67-98 of 3h-56-269); FR4=RGQGTLVTVSS (amino acids 106-116 of 3h-56-269). These sequences correspond to SEQ ID NOs: 42, 44, 47 and 54, respectively, in Table 3. Other exemplary framework regions are shown in Table 3.

TABLE 3

| | | SEQ |
|---|---|---|
| Frame- | | ID |
| work | Sequence | NO: |
| FR1 | EVQLLESGGGLVQPGGSLRLSCAASGFTFR | 42 |
| | EVQLLESGGGLVQPGGSLRLSCAASGFTFA | 43 |
| FR2 | WVRQAPGKGLERVS | 44 |
| | WVRRAPGKGLERVS | 45 |
| | WFRQAPGKGLERVS | 46 |
| FR3 | RFTISRDNSKNTLYLQMNSLRAEDTAVYYCAK | 47 |
| | RFTISRDNSKNMLYLQMNSLRAEDTAVYYCAK | 48 |
| | RFTISRDNSKNTLYLQMNSLRAEDTAVYHCAK | 49 |
| | RFTISRDNSKNTLYLQMNSLRAEDTAVYYCTK | 50 |
| | RFTISRDNSKNTLYLQMNSLRAEDTAVYYCLK | 51 |
| | RFTISRDNSKNTLYLQMNSLRAEDTSVYHCAK | 52 |
| | RFTISRDNSKNTLYLQMNSLRADDTAVYHCTK | 53 |
| FR4 | RGQGTLVTVSS | 54 |
| | WGQGTLVTVSS | 55 |
| | WGQGTLVTVSN | 56 |

Other exemplary framework regions are those of the 3h-56-269 lineage clones disclosed in U.S. Publication No. 2014-0099317.

Anti-CD40 antibody polypeptides comprising the mutated IgG1 Fc domain have therapeutic value in the treatment or prevention of an immune disease. Bringing a protein therapeutic to market requires the molecule to have suitable physical and chemical properties for development, commonly referred to as Chemistry Manufacturing and Control (CMC). The physical and chemical properties of the molecule, including stability, solubility, and homogeneity, are also collectively referred to as “developability”. Advantageously, anti-CD40 antibody polypeptides comprising the mutated IgG1 Fc domain exhibit improved developability, compared to the same anti-CD40 variable domain linked to other IgF1 and IgF4 Fc domains. Anti-CD40 antibody polypeptides comprising the mutated IgG1 Fc domain exhibit reduced binding to Fc gamma receptors, as measured by SPR, and exhibit reduced or undetectable iDC activation as measured by at least one of: 1) release of cytokine IL-6 and/or TNF-alpha; and 2) upregulation of cell surface expression of CD86 and/or CD54. Additionally, anti-CD40 antibody polypeptides comprising the mutated IgG1 Fc domain have improved thermal stability, as measured by DSC, as well as improved physical stability, as measured under accelerated stress conditions. Anti-CD40 antibody polypeptides comprising the mutated IgG1 Fc domain have improved homogeneity.

### 2.3. Linkers

In one embodiment, antibody polypeptides of a fusion antibody polypeptide may be linked by an “amino acid linker” or “linker.” For example, a dAb may be fused to the N-terminus of an amino acid linker, and an Fc domain may be fused to the C-terminus of the linker. Although amino acid linkers can be any length and consist of any combination of amino acids, the linker length may be relatively short (e.g., five or fewer amino acids) to reduce interactions between the linked domains. The amino acid composition of the linker also may be adjusted to reduce the number of amino acids with bulky side chains or amino acids likely to introduce secondary structure. Suitable amino acid linkers include, but are not limited to, those up to 3, 4, 5, 6, 7, 10, 15, 20, or 25 amino acids in length. The linker AST (SEQ ID NO: 57) can be used in the fusion polypeptides. Other representative amino acid linker sequences include GGGGS (SEQ ID NO: 58), and linker comprising 2, 3, 4, or 5 copies of GGGGS (SEQ ID NOs: 59-62, respectively). Table 4 lists exemplary linker sequences for use in the present disclosure.

TABLE 4

| | |
|---|---|
| AST | SEQ ID NO: 57 |
| GGGGS | SEQ ID NO: 58 |
| (GGGGS)₂ | SEQ ID NO: 59 |
| (GGGGS)₃ | SEQ ID NO: 60 |
| (GGGGS)₄ | SEQ ID NO: 61 |
| (GGGGS)₅ | SEQ ID NO: 62 |
| TVAAPS | SEQ ID NO: 63 |
| TVA | SEQ ID NO: 73 |
| ASTSGPS | SEQ ID NO: 74 |

### 2.4 Exemplary Antibody Polypeptides

An exemplary antibody polypeptide comprises: (1) a single variable domain, said single variable domain comprising: (a) a CDR1 region comprising the amino acid sequence of SEQ ID NO: 1 or differing from the CDR1 region of SEQ ID NO: 1 by up to two amino acids, (b) a CDR2 region comprising the amino acid sequence of SEQ ID NO: 2 or differing from the CDR2 region of SEQ ID NO: 2 by up to three amino acids, and (c) a CDR3 region comprising the amino acid sequence of SEQ ID NO: 3 or differing from the CDR3 region of SEQ ID NO: 3 by up to six amino acids, and wherein said single variable domain binds CD40; and (2) an Fc domain that is a human IgG1 Fc domain polypeptide comprising a mutation at Kabat position 238 that reduces binding to Fc-gamma-receptors, wherein proline 238 (P238) is mutated to one of the residues selected from lysine, serine, alanine, arginine and tryptophan. The single variable domain of the antibody polypeptide described herein antagonizes at least one activity of CD40. The antibody polypeptide as described herein has increased stability, relative to a reference polypeptide that has the same single variable domain sequence that is fused to a wild-type IgG1 Fc domain. Provided is an antibody polypeptide comprising: (1) a single variable domain as described above, wherein the human IgG1 Fc domain has a lysine substituted at Kabat position 238.

Exemplary amino acids sequences for the human IgG1 Fc domain polypeptide are:

EPKSCDKTHTCPPCPAPELLGGKSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG (IgG1a-P238K; SEQ
ID NO: 134),
EPKSCDKTHTCPPCPAPELLGGKSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK (IgG1a-P238K; SEQ
ID NO: 66),
EPKSCDKTHTCPPCPAPELLGGKSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPG (IgG1f-P238K; SEQ
ID NO: 135),
and
EPKSCDKTHTCPPCPAPELLGGKSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK (IgG1f-P238K; SEQ
ID NO: 67).

An exemplary antibody polypeptide is as described above, wherein (a) the CDR1 region consists of a sequence X1-Tyr-Glu-Y1-Trp (SEQ ID NO: 4), wherein X1 is Asp or Gly, and Y1 is Met or Leu; (b) the CDR2 region consists of a sequence Ala-Ile-Asn-Pro-X2-Gly-Y2-Z2-Thr-Tyr-Tyr-Ala-Asp-Ser-Val-A2-Gly (SEQ ID NO: 5), wherein X2 is Gln, Tyr, His, Trp, or Ala, Y2 is Thr, Asn, Gly, Ser, or Gln, Z2 is Arg, Leu, Tyr, His, or Phe, and A2 is Lys or Met; and (c) the CDR3 region consists of a sequence X3-Pro-Y3-Z3-A3-B3-C3 (SEQ ID NO: 6), wherein X3 is Leu, Pro, or Glu, Y3 is Phe, Gln, Thr, Met, or Tyr, Z3 is Arg, Tyr, Pro, Leu, Thr, Ile, Phe, Met, or Ser, A3 is Phe or Tyr, B3 is Ser, Gln, His, Asp, Lys, Glu, or Gly, and C3 is Asp, Tyr, Glu, or Ser.

An exemplary antibody polypeptide is as described above, wherein: (a) the CDR1 region consists of the amino acid sequence of SEQ ID NO: 1 (CDR1 of 3h-56-269), (b) the CDR2 region consists of the amino acid sequence of SEQ ID NO: 2 (CDR2 of 3h-56-269), and (c) the CDR3 region consists of the amino acid sequence of SEQ ID NO: 3 (CDR3 of 3h-56-269). Further provided is antibody polypeptide as described above, wherein the amino acid sequence of the single variable domain is set forth in SEQ ID NO: 41 (=3h-56-269 sequence).

An exemplary antibody polypeptide comprises or consists of the amino acid sequence:

(SEQ ID NO: 70)
EVQLLESGGGLVQPGGSLRLSCAASGFTFRDYEMWWVRQAPGKGLERVSA
INPQGTRTYYADSVKGRFTISRDNSKNTLYLQMNSLRAEDTAVYYCAKLP
FRFSDRGQGTLVTVSSASTEPKSCDKTHTCPPCPAPELLGGKSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

An exemplary antibody polypeptide comprises or consists of the amino acid sequence:

(SEQ ID NO: 136)
EVQLLESGGGLVQPGGSLRLSCAASGFTFRDYEMWWVRQAPGKGLERVSA
INPQGTRTYYADSVKGRFTISRDNSKNTLYLQMNSLRAEDTAVYYCAKLP
FRFSDRGQGTLVTVSSASTEPKSCDKTHTCPPCPAPELLGGKSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
G.

An exemplary antibody polypeptide comprises or consists of the amino acid sequence:

(SEQ ID NO: 71)
EVQLLESGGGLVQPGGSLRLSCAASGFTFRDYEMWWVRQAPGKGLERVSA
INPQGTRTYYADSVKGRFTISRDNSKNTLYLQMNSLRAEDTAVYYCAKLP
FRFSDRGQGTLVTVSSASTEPKSCDKTHTCPPCPAPELLGGKSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

An exemplary antibody polypeptide comprises or consists of the amino acid sequence:

(SEQ ID NO: 137)
EVQLLESGGGLVQPGGSLRLSCAASGFTFRDYEMWWVRQAPGKGLERVSA
INPQGTRTYYADSVKGRFTISRDNSKNTLYLQMNSLRAEDTAVYYCAKLP
FRFSDRGQGTLVTVSSASTEPKSCDKTHTCPPCPAPELLGGKSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSP
G.

### 2.5. Antibody Polypeptide Preparation

The antibody polypeptides of the disclosure can be produced and purified using only ordinary skill in any suitable mammalian host cell line, such as CHO, HEK293, COS, NSO, and the like, followed by purification using one or a combination of methods, including protein A affinity chromatography, ion exchange, reverse phase techniques, or the like.

The disclosure further provides a nucleic acid encoding the antibody polypeptide of disclosure. The nucleic acid may be inserted into a vector, such as a suitable expression vector, e.g., pHEN-1 (Hoogenboom et al. (1991) Nucleic Acids Res. 19:4133-4137). Further provided is an isolated host cell comprising the vector and/or the nucleic acid encoding the disclosed antibody polypeptides.

### 3. Pharmaceutical Compositions and Methods of Treatment

A pharmaceutical composition comprises a therapeutically-effective amount of one or more antibody polypeptides and optionally a pharmaceutically acceptable carrier. Pharmaceutically acceptable carriers include, for example, water, saline, phosphate buffered saline, dextrose, glycerol, ethanol and the like, as well as combinations thereof. Pharmaceutically acceptable carriers can further comprise minor amounts of auxiliary substances, such as wetting or emulsifying agents, preservatives, or buffers that enhance the shelf-life or effectiveness of the fusion protein. The compositions can be formulated to provide quick, sustained, or delayed release of the active ingredient(s) after administration. Suitable pharmaceutical compositions and processes for preparing them are well known in the art. See, e.g., Remington, The Science and Practice of Pharmacy, A. Gennaro, et al., eds., 21st ed., Mack Publishing Co. (2005).

The pharmaceutical composition further may comprise an immuno-suppressive/immunomodulatory and/or anti-inflammatory agent.

A method of treating an immune disease in a patient in need of such treatment may comprise administering to the patient a therapeutically effective amount of the pharmaceutical composition. Antagonizing CD40-mediated T cell activation could inhibit undesired T cell responses occurring during autoimmunity, transplant rejection, or allergic responses, for example. Inhibiting CD40-mediated T cell activation could moderate the progression and/or severity of these diseases.

Also provided is the use of an antibody polypeptide of the disclosure, or a pharmaceutically acceptable salt thereof, in the preparation of a medicament for treatment of an immune disease in a patient in need of such treatment. The medicament can, for example, be administered in combination with an immunosuppressive/immunomodulatory and/or anti-inflammatory agent.

As used herein, a “patient” means an animal, e.g. mammal, including humans. The patient may be diagnosed with an immune disease. “Treatment” or “treat” or “treating” refers to the process involving alleviating the progression or severity of a symptom, disorder, condition, or disease. An “immune disease” refers to any disease associated with the development of an immune reaction in an individual, including a cellular and/or a humoral immune reaction. Examples of immune diseases include, but are not limited to, inflammation, allergy, autoimmune disease, or graft-related disease. An “autoimmune disease” refers to any disease associated with the development of an autoimmune reaction in an individual, including a cellular and/or a humoral immune reaction. An example of an autoimmune disease is inflammatory bowel disease (IBD), including, but not limited to ulcerative colitis and Crohn's disease. Other autoimmune diseases include systemic lupus erythematosus, multiple sclerosis, rheumatoid arthritis, diabetes, psoriasis, scleroderma, and atherosclerosis. Graft-related diseases include graft versus host disease (GVHD), acute transplantation rejection, and chronic transplantation rejection.

Diseases that can be treated by administering the pharmaceutical composition of the disclosure may be selected from the group consisting of Addison's disease, allergies, anaphylaxis, ankylosing spondylitis, asthma, atherosclerosis, atopic allergy, autoimmune diseases of the ear, autoimmune diseases of the eye, autoimmune hepatitis, autoimmune parotitis, bronchial asthma, coronary heart disease, Crohn's disease, diabetes, epididymitis, glomerulonephritis, Graves' disease, Guillain-Barre syndrome, Hashimoto's disease, hemolytic anemia, idiopathic thrombocytopenia purpura, inflammatory bowel disease, immune response to recombinant drug products (e.g., Factor VII in hemophiliacs), systemic lupus erythematosus, multiple sclerosis, myasthenia gravis, pemphigus, psoriasis, rheumatic fever, rheumatoid arthritis, sarcoidosis, scleroderma. Sjogren's syndrome, spondyloarthropathies, thyroiditis, transplant rejection, vasculitis, and ulcerative colitis.

The pharmaceutical composition may be administered alone or in combination therapy, (i.e., simultaneously or sequentially) with an immunosuppressive/immunomodulatory and/or anti-inflammatory agent. Different immune diseases can require use of specific auxiliary compounds useful for treating immune diseases, which can be determined on a patient-to-patient basis. For example, the pharmaceutical composition may be administered in combination with one or more suitable adjuvants, e.g., cytokines (IL-10 and IL-13, for example) or other immune stimulators, e.g., chemokines, tumor-associated antigens, and peptides. Suitable adjuvants are known in the art.

Any suitable method or route can be used to administer the antibody polypeptide or the pharmaceutical composition. Routes of administration include, for example, oral, intravenous, intraperitoneal, subcutaneous, or intramuscular administration. A therapeutically effective dose of administered antibody polypeptide(s) depends on numerous factors, including, for example, the type and severity of the immune disease being treated, the use of combination therapy, the route of administration of the antibody polypeptide(s) or pharmaceutical composition, and the weight of the patient. A non-limiting range for a therapeutically effective amount of a domain antibody is 0.1-20 mg/kg, and in an aspect, 1-10 mg/kg, relative to the body weight of the patient.

### 4. Kits

A kit useful for treating an immune disease in a human patient is provided. In an embodiment, the kit comprises (a) a dose of an antibody polypeptide of the present disclosure and (b) instructional material for using the antibody polypeptide in the method of treating an immune disease in a human patient as disclosed herein.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

### EXAMPLES

Materials and Methods: This section describes materials and methods used in the following examples. Additional methods are disclosed in the examples.

Proteins: Antibodies and dAb-Fc proteins were expressed in either HEK293 (cell line derived from human embryonic kidney cells) or Expi293 cells, and purified by standard protein A affinity chromatography, followed by preparative size exclusion chromatography. A few select samples were also expressed and purified from UCOE-CHO cells (samples indicated with “UCOE-CHO”).

CD40 Binding Kinetics and Affinity: The CD40 binding affinity of dAb-Fc and antibody molecules was measured by SPR on a Biacore™ T100 or T200 instrument (GE Healthcare Life Sciences, Marlborough, MA) by capturing a dAb-Fc or an antibody on an immobilized protein A sensor chip surface, and then binding human-CD40-monomer protein (generated in house) using an association time of 180 seconds, dissociation time of 360 seconds at 30 microliter per minute (μl/min) in PBS-T PH 7.1. To characterize the binding with avidity, human-CD40-Fc (generated in house) was immobilized on a CM5 sensor chip, and dAb-Fc or antibody analytes were tested for binding using 180 second association time and 240 second dissociation time at 30 μl/min.

### Example 1: Treatment of iDCs with dAb-Fc Molecules in the Presence or Absence of FcγR Crosslinking

3h56-269-IgG4.1 is an anti-CD40 dAb-FC (IgG4) fusion protein (SEQ ID NO: 75). No direct agonist activities have been observed for 3h56-269-IgG4.1 in B cells or T-cell-depleted peripheral blood mononuclear cells (PBMCs), as described for instance in WO 2012/145673. To further characterize the biological activity and safety profile of 3h56-269-IgG4.1, the effect of 3h56-269-IgG4.1 on immature dendritic cells (iDC) was assayed.

Materials and Methods used in this example include the following:

Primary Cell Isolation and Culture: Peripheral blood was collected from normal, healthy human donors. Peripheral blood mononuclear cells (PBMC) were isolated from heparinized human blood by Ficoll density gradient separation. Monocytes were isolated from PBMC following the Manual EasySep protocol (STEMCELL™ Technologies, Vancouver, Canada). One million isolated monocytes were plated in each well of a 6-well plate in 6 ml of complete media (RPMI-1640, 10% Heat inactivated Fetal Bovine Serum, 100 Units/ml Penicillin-Streptomycin), further containing IL-4 (100 nanogram per milliliter (ng/ml)) and human GM-CSF (100 ng/ml) and incubated for 6 days at 37° C. and 5% CO2. Media was changed every other day and replaced with fresh media containing the same concentration of cytokines. Immature dendritic cells (iDCs) were harvested by centrifugation on day 6, washed thoroughly, and re-suspended in complete media.

Immature Dendritic Cell Activation Assay: Immature Dendritic Cells (iDCs) were assayed for activation by assessing release of specific cytokines and expression of specific cell surface molecules. Titrations of the various biological agents were made in complete media, and added to duplicate 96-well plates. In the case of cross-linking (via addition of CD32a-expressing CHO cells), antibodies being assayed were added to the iDCs for 30 minutes prior to the addition of CD32a-expressing CHO cells. The ratio of CD32a-expressing CHO cells to iDCs was 1:6.

To assess cytokines, cells were incubated at 37° C. and 5% CO2 for approximately 18-20 hours: 150 microliter (μL) of supernatant was removed from each well, diluted 1:5 and evaluated for protein concentrations of IL-6, TNF& and IL-12 using a commercially available ELISA kits (R&D Systems, Minneapolis, MN), according to manufacturer's instructions.

To assess CD86, ICAM-1 (also called CD54) and CD83 expression, the cells remaining in the plates from the harvested supernatants were combined into 1 sample per duplicate treatment, and transferred to a new 96-well round bottom (RB) plate, and placed at 4° C. Cells were washed with D-PBS, Ca++ and Mg++ free, and stained for 30 min on ice for cell viability using the LIVE/DEAD® Fixable Near-IR Dead Cell Stain Kit (Invitrogen, Carlsbad, CA). Cells were washed and resuspended in D-PBS, Ca++ and Mg++ free, 2% FBS, 0.1% NaN3 (staining buffer) and blocked with 5 L/well of Human TruStain FcX™ (Fc Receptor Blocking Solution, Biolegend, San Diego, CA) in staining buffer. The iDCs were immuno-stained with: PerCpCy5.5-conjugated αCD3, αCD19, αCD14 (Lin−), BUV395-conjugated αCD11c (BD Biosciences, San Diego, CA), APC-conjugated αCD86 (Biolegend, San Diego, CA), PE-conjugated αCD83 (eBioscience, San Diego, CA), FITC-conjugated αCD54 (Biolegend, San Diego, CA), and incubated at 4° C. for 45 minutes. Cells were then washed twice in staining buffer and fixed (15 minutes at room temperature (RT), protected from light), by adding 100 μl of BD Cytofix Fixation Buffer (BD Bioscience, San Diego, CA). The iDCs were evaluated for CD86, ICAM-1 and CD83 expression using a LSRII-Fortessa™ Flow Cytometer (BD Biosciences, San Diego, CA), and FlowJo® analysis software (Tree Star Inc., Ashland, OR).

CP-870,893 mAb is a well-known agonistic CD mAb (see, e.g., Vonderheide et al., 2007, J. Clin. Oncol. 25 (7): 876-883). In these studies, CP-870,893 mAb (referred to herein as mAb 134-2141; generated in house), served as a positive control. A second positive control was a soluble CD40L trimer molecule (generated in house) that is trimerized by an isoleucine zipper trimerization motif. In some experiments, CH1-L6 IgG4 (generated in house), a fusion protein between a non-CD40 binding protein and an IgG4.1 Fc tail, served as a negative control.

dAb-Fcs: The amino acid sequences of the dAb-Fes studied in this experiment are shown in Table 5. In these sequences, the single variable domain 3h56-269 residues are amino acids 1-118 (underlined). The linker, AST (SEQ ID NO: 57), is double-underlined. The unformatted C-terminal residues are the Fc domain.

TABLE 5

| SEQ ID | | |
|---|---|---|
| NO: | Sample ID | Sequence |
| 75 | 3h56-269- | EVQLLESGGGLVQPGGSLRLSCAASGFTFRDYEMWWVRQAPGKGLERVSA |
| | IgG4.1 | INPQGTRTYYADSVKGRFTISRDNSKNTLYLQMNSLRAEDTAVYYCAKLP |
| | OR | FRFSDRGQGTLVTVSSASTESKYGPPCPPCPAPEFLGGPSVFLFPPKPKD |
| | BMS-986090 | TLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVY |
| | | TLPPSQEEMTKNQVSLTCLVKGFYPSDIAVEWESNGQPENNYKTTPPVLD |
| | | SDGSFFLYSRLTVDKSRWQEGNVFSCSVMHEALHNHYTQKSLSLSLGK |
| 76 | 3h56-269-CT | EVQLLESGGGLVQPGGSLRLSCAASGFTFRDYEMWWVRQAPGKGLERVSA |
| | | INPQGTRTYYADSVKGRFTISRDNSKNTLYLQMNSLRAEDTAVYYCAKLP |
| | | FRFSDRGQGTLVTVSSASTEPKSSDKTHTSPPSPAPELLGGSSVFLFPPK |
| | | PKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |

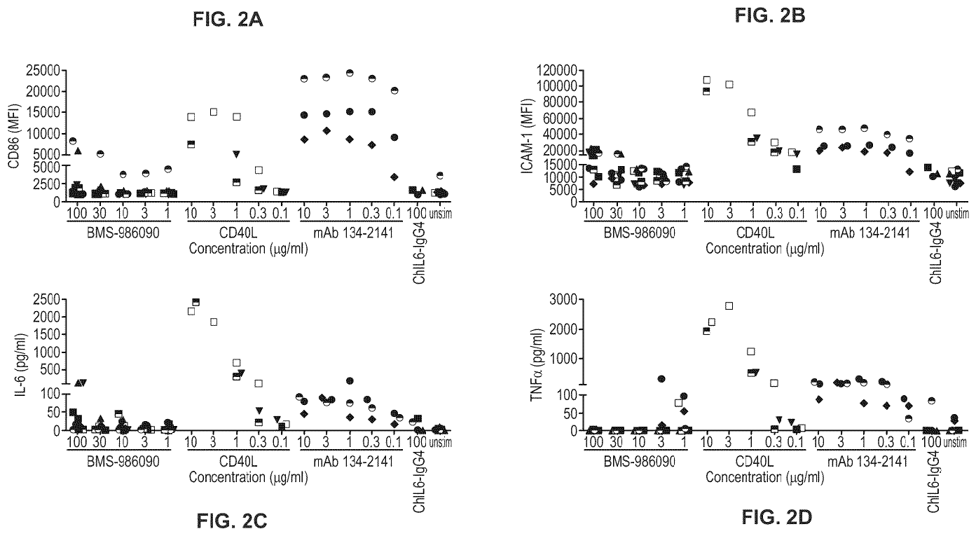

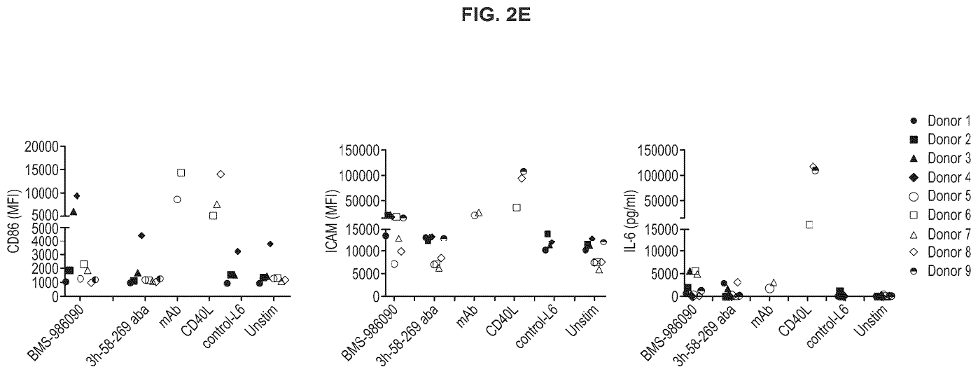

Results: The effect of 3h56-269-IgG4.1 on immature dendritic cells (iDC) was assayed. Up-regulation of CD86 and ICAM-1 (CD54) expression as well cytokine release (e.g., IL-6, TNF) was evaluated. The assay was performed on iDCs from 9 different donors. A modest increase in CD86 expression on iDCs was observed with 3h56-269-IgG4.1 in a 1 of 9 donors at 30 μg/ml and 2 of 9 donors as 100 μg/ml, with a similarly modest increase in cytokine release was observed in 1 of 9 donors. See FIGS. 2A-2D. Thus, it was concluded that the Fc-anti-CD40 dAb fusion 3h56-269-IgG4.1 activated immature dendritic cells (iDCs) in a small subset of donors.

Immature DC express both FcgR and CD40 and are sensitive to CD40 activation. Therefore the potential that FcgR mediated clustering or cross-linking could be responsible for the observed iDC activation with 3h56-269-IgG4.1 was explored. 3h-56-269-CT (SEQ ID NO: 76) is a fusion of the same anti-CD40 dAb (3h-56-269) to an IgG1 Fc tail with reduced FcgR binding referred to herein as “CT” or “aba”. The CT Fc domain is the Fc domain present in Orencia® (abatacept, Bristol-Myers Squibb Company, New York, NY). Abatacept is a fusion of the extracellular domain of CTLA-4 to an IgG1 Fc domain that is modified to reduce Fc domain effector function and eliminate interchain disulfide bonds in the IgG1 hinge region. 3h-56-269-CT has reduced FcgR binding.

3h-59-269-CT was tested at 100 g/ml with iDC from 9 donors. The 9 donor iDCs showed neither cytokine release nor upregulation of CD86 or CD54 when compared to the negative control consisting of CH1-L6 IgG4, a fusion protein between a non-CD40 protein and an IgG4 Fc tail. In contrast, 3h56-269-IgG4.1 exhibited iDC activation in a subset of 3 of the 9 donors in which at least one measure of iDC activation was observed to be greater than control. The CD40 agonist mAb 134-214 stimulated CD86 and ICAM expression and cytokine release in all donors tested. See FIG. 2E.

These data suggest a role for the Fc portion of the fusion protein in the iDC activation. Specifically, these observations suggest that FcgR clustering or crosslinking by the IgG4.1 Fc domain of 3h56-269-IgG4.1 on the surface of iDC may account for the activation observed in a subset of donors. The reduced iDC activation of the 3h-59-269-CT fusion protein is consistent with the reduced binding to the FcgR receptors, including CD32 (FcgRII) and CD16 (FcgRIII), as assessed by surface plasmon resonance (SPR).

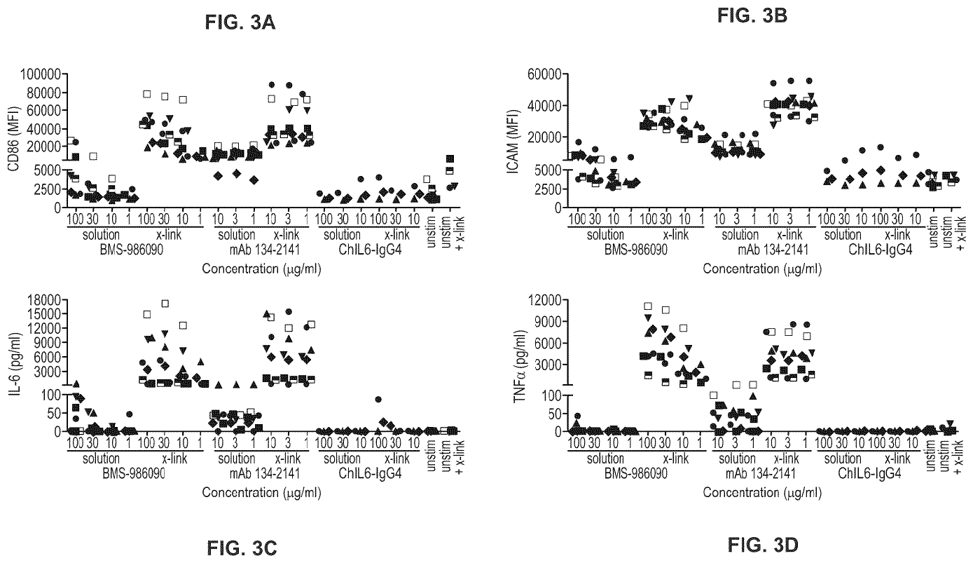

To further explore the impact of FcgR-mediated dAb cross-linking on cell surface marker expression and cytokine release of iDCs, additional experiments were conducted across 8 blood donors, in which CHO cells highly over-expressing CD32a, the low affinity FcgR, were used to cluster/cross-link 3h56-269-IgG4.1. It should be noted that the ratio of CHO cells to iDCs in these experiments was 1:6. This ratio is an exaggerated level of clustering/cross-linking, and likely above what would be expected to occur under normal physiological conditions. Similar to what was observed in the previous iDC study (FIGS. 2A-2E), 3h56-269-IgG4.1, in the absence of cross-linking, caused a modest amount of iDC activation as measured by CD86 and IL-6 production in a few donors when compared to the CH1-L6 IgG4 control. In contrast, when CD32 over-expressing CHO cells were incorporated, 3h56-269-IgG4.1 at concentrations≥10 μg/ml resulted in iDC activation similar to that of cross-linking the agonist CD40 antibody, as measured by both cell surface markers and cytokine release (FIGS. 3A-3D).

### Example 2: dAB-Fc Molecules with Compromised FcgR Binding

To determine whether additional Fc mutations could reduce the indirect iDC activation mediated by FcgR cluster or cross-linking, other dAb-Fc molecules were produced with mutations in the Fc domain to reduce FcgR binding. The FcgR binding affinities were characterized by SPR. Materials and Methods used in this example include the following.

FcgR Binding SPR: FcgR binding can be measured in vitro using purified FcgRs using Biacore™ surface plasmon resonance (SPR). Two methods were used herein.

One method tests the binding of purified antibodies or dAb-Fc proteins to His-tagged FcgR proteins (FcgR-His) which are captured on the immobilized Fab fragment of an anti-His antibody. These experiments are performed on either a Biacore™ T100 or Biacore™ T200 instrument (GE Healthcare) at 25° C. The Fab fragment from a murine anti-6×His antibody (generated in house) is immobilized on a CM5 sensor chip using standard ethyl(dimethylaminopropyl) carbodiimide (EDC)/N-hydroxysuccinimide (NHS) chemistry with ethanolamine blocking, to a density of ˜3000 Resonance Units RU in a running buffer of millimolar (mM) HEPES pH 7.4, 150 mM NaCl, 3 mM EDTA, 0.05% surfactant p20 (HBS-EP+). All remaining studies are performed using a running buffer of 10 mM NaPO4, 130 mM NaCl, 0.05% p20 (PBS-T) at pH 7.1. Various FcgR proteins containing a C-terminal 6× poly-histidine tag (generated in house) were captured on this surface (typically using FcgR-His protein concentration of ˜7 μg/ml) using a contact time of 30 seconds(s) at 10 μl/min. Various concentrations of purified antibody or dAb-Fc proteins are tested for binding, for example using an association time of 120 seconds at 30 μl/min, and a dissociation time of 120 seconds at μl/min. FcgR proteins tested in these studies include the “high affinity” FcgR hCD64 (hFcgRI), as well as the “low affinity” FcgRs hCD32a-H131 (FcgRIIa-H131), hCD32a-R131 (FcgRIIa-R131), hCD32b (FcgRIIb), hCD16a-V158 (FcgRIIIa-V158), hCD16a-F158 (FcgRIIIa-F158), hCD16b-NA1 (FcgRIIIb-NA1), and hCD16b-NA2 (FcgRIIIb-NA2).

To quantitatively analyze the binding responses and compare the FcgR binding of different molecules, the SPR binding data can be analyzed by calculating the maximum binding response as a percentage of the theoretical maximum binding response (% Rmax) as generally shown in Eq. 1:

$$\% R max = \frac{( Observed Binding Response Analyte )}{( Theoretical Maximum Binding Response Analyte )} \qquad Eq . 1$$

Specifically, the % Rmax is calculated using the equation:

$$\% R max = \frac{( Binding Response Analyte )}{\begin{matrix}\frac{( Mw Analyte )}{( Mw Ligand )} \times \\ ( Response Ligand ) \times ( analyte : ligand stoichiometry )\end{matrix}} \qquad Eq . 2$$

where “Analyte” is the antibody or dAb-Fc and “Ligand” is the captured FcgR protein. This analysis does not take into account the mass of glycosylation of antibody, dAb-Fc or FcgR, and assumes 100% fractional activity for the captured ligand.

The “% Rmax analysis” is particularly useful for evaluating the binding of the “low affinity” FcgRs, e.g., hCD32a-H131, hCD32a-R131, hCD32b, hCD16a-V158, hCD16a-F158, hCD16b-NA1, and hCD16b-NA2, which have relatively fast association and dissociation rates and affinities near or below the analyte concentration tested (1 micromolar (μM)), so saturation of the surface is generally not achieved under these conditions. In contrast, the “high affinity” FcgR hCD64 binds with higher affinity and slower dissociation kinetics than the other FcgRs, particularly to IgG1 and IgG4, and thus these isotypes do typically saturate the hCD64 surface under micromolar analyte concentrations, and are more difficult to differentiate affinities using % Rmax. For these interactions, differences between antibodies can be easily observed by comparison of the dissociation rates in the sensorgram data.

A second SPR assay for testing the interaction between antibodies or dAb-Fc proteins with FcgR proteins is a protein A capture method. These experiments are also performed on either a Biacore™ T100 or Biacore™ T200 instrument (GE Healthcare) at 25° C. For these studies, protein A is immobilized on flow cells 1-4 of a CM5 sensor chip using standard ethyl(dimethylaminopropyl) carbodiimide (EDC)/N-hydroxysuccinimide (NHS) chemistry, with ethanolamine blocking, in a running buffer of 10 mM HEPES pH 7.4, 150 mM NaCl, 3 mM EDTA, 0.05% surfactant p20, to a density of ˜3000 RU. Antibody or dAb-Fc proteins (typically ˜3-10 μg/ml) are captured on the protein A surface, and the binding of FcgR analytes are tested in running buffer consisting of 10 mM NaPO4, 130 mM NaCl, 0.05% p20, buffer (PBS-T) at pH 7.1 and at 25° C., using for example, 120 sec association time and 180 sec dissociation time at a flow rate of 30 μL/min.

The protein A capture assay can also be used to analyze unpurified supernatants containing antibody or dAb-Fc molecules. For this analysis, the antibody or dAb-Fc proteins can be captured from either undiluted supernatants or supernatants diluted with running buffer. To quantitatively analyze the binding responses and compare the FcgR binding of different molecules, the SPR binding data can be analyzed by calculating the % Rmax using Eq. 1 above, wherein Analyte is the purified FcgR protein, and Ligand is the captured antibody or dAb-Fc protein.

In addition to % Rmax analysis, quantitative analysis of the kinetics and affinity of binding can be performed by testing a titration of FcgR analyte for binding to protein A captured antibodies or dAb-Fc proteins. For example, FcgR in a 3:1 serial dilution can be titrated from 10 μM down to either 0.15 nM (hCD64) or 1.5 nM (all other FcgRs). These kinetic data can be fit to either a 1:1 Langmuir model or to a steady-state binding model using Biacore™ T200 evaluation software to obtain kinetic and affinity values.

dAb-Fcs: The dAb-Fcs studied in this example include those shown in Table 5. The amino acid sequences of the additional dAb-Fcs studied in this experiment are shown in Table 6. In these sequences, the single variable domain 3h56-269 residues are amino acids 1-118 (underlined). The linker AST (SEQ ID NO: 57) is double-underlined. The C-terminal residues are the Fc domain.

TABLE 6

| SEQ | | |
|---|---|---|
| ID | dAb-FC sample | |
| NO: | name | Sequence |
| 77 | 3h56-269-IgG1.1f | EVQLLESGGGLVQPGGSLRLSCAASGFTFRDYEMWWVRQAPGKGLERVSA |
| | | INPQGTRTYYADSVKGRFTISRDNSKNTLYLQMNSLRAEDTAVYYCAKLPFRF |
| | | SDRGQGTLVTVSSASTEPKSCDKTHTCPPCPAPEAEGAPSVFLFPPKPKDTL |
| | | MISRTPEVTCVVVDVSHEDPEVKFNWYVDGVEVHNAKTKPREEQYNSTYRV |
| | | VSVLTVLHQDWLNGKEYKCKVSNKALPSSIEKTISKAKGQPREPQVYTLPPSR |
| | | EEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 78 | 3h56-269-IgG1.3f | EVQLLESGGGLVQPGGSLRLSCAASGFTFRDYEMWWVRQAPGKGLERVSA |
| | | INPQGTRTYYADSVKGRFTISRDNSKNTLYLQMNSLRAEDTAVYYCAKLPFRF |
| | | SDRGQGTLVTVSSASTEPKSCDKTHTCPPCPAPEAEGAPSVFLFPPKPKDTL |
| | | MISRTPEVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 79 | 3h56-269-IgG1- | EVQLLESGGGLVQPGGSLRLSCAASGFTFRDYEMWWVRQAPGKGLERVSA |
| | D265A | INPQGTRTYYADSVKGRFTISRDNSKNTLYLQMNSLRAEDTAVYYCAKLPFRF |
| | | SDRGQGTLVTVSSASTEPKSCDKTHTCPPCPAPELLGGPSVFLFPPKPKDTLM |
| | | ISRTPEVTCVVVAVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |

Control mAb: A control monoclonal antibody (1F4) was also formatted with similar Fc domain mutations. The antibody does not bind to CD40. SEQ ID NO: 80 in Table 7 is the sequence of the control antibody heavy chain variable region (underlined) and CH1, and SEQ ID NO: 81 is the sequence of light chain variable region (underlined) and CL. The various formatted heavy chains are shown in Table 7 as SEQ ID NOS; 82-87. The IF4 heavy chain variable region and CH1 region sequence is underlined in SEQ ID NOS: 82-87. The pair of heavy chain and light chain sequences for each 1F4 mAb variant is shown in Table 8.

TABLE 7

| SEQ | | |
|---|---|---|
| ID | | |
| NO: | Sequence identity | Sequence |
| 80 | 1F4 Heavy chain | EVQLLESGGGLVQPGGSLRLSCAASGFTFSIYAMSWVRQAPG |
| | variable region | GKLEWVSAISDSGGRTYFADSVRGRFTISRDNSKNTLSLQMNS |
| | and CH1 | LRAEDTAVYYCAKVDYSNYLFFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKRV |
| 81 | 1F4 Light chain | EIVLTQSPGTLSLSPGERATLSCRASQSISSSYLAWYQQKPGQA |
| | variable region | PRLLIYGASSRATGIPDRFSGSGSGTDFTLTISRLEPEDFAVYYC |
| | and CL | QQYGSSPYTFGQGTKLEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| 82 | 1F4-IgG1f heavy | EVQLLESGGGLVQPGGSLRLSCAASGFTFSIYAMSWVRQAPG |
| | chain | KGLEWVSAISDSGGRTYFADSVRGRFTISRDNSKNTLSLQMNS |
| | | LRAEDTAVYYCAKVDYSNYLFFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKRVEPKSCDKTHTCPPCPAPELLGGPSVFLFPPKPKDTLM |
| | | ISRTPEVTCVVVDVSHEDPEVKFNWYVDGVEVHNAKTKPREE |
| | | QYNSTYRVVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 83 | 1F4-IgG4.1 heavy | EVQLLESGGGLVQPGGSLRLSCAASGFTFSIYAMSWVRQAPG |
| | chain | KGLEWVSAISDSGGRTYFADSVRGRFTISRDNSKNTLSLQMNS |
| | | LRAEDTAVYYCAKVDYSNYLFFDYWGQGTLVTVSSASTKGP |
| | | SVFPLAPCSRSTSESTAALGCLVKDYFPEPVTVSWNSGALTSG |
| | | VHTFPAVLQSSGLYSLSSVVTVPSSSLGTKTYTCNVDHKPSNT |
| | | KVDKRVESKYGPPCPPCPAPEFLGGPSVFLFPPKPKDTLMISRT |
| | | PEVTCVVVDVSQEDPEVQFNWYVDGVEVHNAKTKPREEQFN |
| | | STYRVVSVLTVLHQDWLNGKEYKCKVSNKGLPSSIEKTISKA |
| | | KGQPREPQVYTLPPSQEEMTKNQVSLTCLVKGFYPSDIAVEW |
| | | ESNGQPENNYKTTPPVLDSDGSFFLYSRLTVDKSRWQEGNVF |
| | | SCSVMHEALHNHYTQKSLSLSLG |
| 84 | 1F4-IgG1.1f | EVQLLESGGGLVQPGGSLRLSCAASGFTFSIYAMSWVRQAPG |
| | heavy chain | KGLEWVSAISDSGGRTYFADSVRGRFTISRDNSKNTLSLQMNS |
| | | LRAEDTAVYYCAKVDYSNYLFFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKRVEPKSCDKTHTCPPCPAPEAEGAPSVFLFPPKPKDTLM |
| | | ISRTPEVTCVVVDVSHEDPEVKFNWYVDGVEVHNAKTKPREE |
| | | QYNSTYRVVSVLTVLHQDWLNGKEYKCKVSNKALPSSIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 85 | 1F4-IgG1.3f | EVQLLESGGGLVQPGGSLRLSCAASGFTFSIYAMSWVRQAPG |
| | heavy chain | KGLEWVSAISDSGGRTYFADSVRGRFTISRDNSKNTLSLQMNS |
| | | LRAEDTAVYYCAKVDYSNYLFFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKRVEPKSCDKTHTCPPCPAPEAEGAPSVFLFPPKPKDTLM |
| | | ISRTPEVTCVVVDVSHEDPEVKFNWYVDGVEVHNAKTKPREE |
| | | QYNSTYRVVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 86 | 1F4-D265A heavy | EVQLLESGGGLVQPGGSLRLSCAASGFTFSIYAMSWVRQAGK |
| | chain | GLEWVSAISDSGGRTYFADSVRGRFTISRDNSKNTLSLQMNSL |
| | | RAEDTAVYYCAKVDYSNYLFFDYWGQGTLVTVSSASTKGPS |
| | | VFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKRVEPKSCDKTHTCPPCPAPELLGGPSVFLFPPKPKDTLM |
| | | ISRTPEVTCVVVAVSHEDPEVKFNWYVDGVEVHNAKTKPREE |
| | | QYNSTYRVVSVLTVLHQDWLNGKEYKCKVSNKALPAPIEKTI |
| | | SKAKGQPREPQVYTLPPSREEMTKNQVSLTCLVKGFYPSDIVE |
| | | WESNGQPENNYKTTPPVLDSDGSFFLYSKLTVDKSRWQQGN |
| | | VFSCSVMHEALHNHYTQKSLSLSPGK |
| 87 | 1F4-CT heavy | EVQLLESGGGLVQPGGSLRLSCAASGFTFSIYAMSWVRQAPG |
| | chain | KGLEWVSAISDSGGRTYFADSVRGRFTISRDNSKNTLSLQMNS |
| | | LRAEDTAVYYCAKVDYSNYLFFDYWGQGTLVTVSSASTKGP |
| | | SVFPLAPSSKSTSGGTAALGCLVKDYFPEPVTVSWNSGALTSG |
| | | VHTFPAVLQSSGLYSLSSVVTVPSSSLGTQTYICNVNHKPSNT |
| | | KVDKRVEPKSCDKTHTSPPSPAPELLGGSSVFLFPPKPKDTLMI |
| | | SRTPEVTCVVVDVSHEDPEVKFNWYVDGVEVHNAKTKPREE |
| | | QYNSTYRVVSVLTVLHQDWLNGKEYKCKVSNKALPAPIEKTI |
| | | SKAKGQPREPQVYTLPPSRDELTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPGK |

TABLE 8

| mAb name | HC | LC |
|---|---|---|
| 1F4-IgG1f | SEQ #: 82 | SEQ #: 81 |
| 1F4-IgG4.1 | SEQ #: 83 | SEQ #: 81 |
| 1F4-IgG1.1f | SEQ #: 84 | SEQ #: 81 |
| 1F4-IgG1.3f | SEQ #: 85 | SEQ #: 81 |
| 1F4-D265A | SEQ #: 86 | SEQ #: 81 |
| 1F4-CT | SEQ #: 87 | SEQ #: 81 |

Results: dAb-Fc molecules were produced with mutations in the Fc domain to reduce FcgR binding. Specifically, the anti-CD40 domain antibody 3h56-269 was formatted with the following Fc domain variants: IgG1.1f, IgG1.3f, and IgG1-D265A. In each of 3h-56-269-IgG1.1f (SEQ ID NO: 77), 3h-56-269-IgG1.3f (SEQ ID NO: 78), and 3h-56-269-IgG1-D265A (SEQ ID NO: 79), amino acids 1-116 are 3h-56-269 dAb, amino acids 117-119 are a linker, and amino acids 120-351 are the Fc domain.

Each these dAb-Fc fusion proteins, as well as each of 3h56-269-IgG4.1 and 3h56-269-CT, was confirmed to bind with high affinity to purified human-CD40 monomer (hCD40monomer, generated in house) as measured by Biacore™ SPR. As shown in Table 9, the KD values range between 7.3 nM and 11.5 nM for the different Fc variants. Each of the dAb-Fc molecules also bound human CD40 with high avidity, as measured by SPR using hCD40-Fc on the surface of a sensor chip and the dAb-Fc molecules as soluble analytes in solution, where data for 250 nM and 25 nM dAb-Fc analyte injections were fit to a 1:1 Langmuir model to estimate avidity-influenced apparent KD values (KDₐₚₚₐᵣₑₙₜ) for all dAb-Fcs as <1 nM. See Table 9.

TABLE 9
SPR data for binding of dAb-Fc molecules to human CD40.

| | hCD40monomer binding to | | | |
|---|---|---|---|---|
| | dAb-Fc molecules captured | | | dAb-Fc binding |
| | on immobilized protein A | | | to immobilized |
| | surface (Affinity) | | | hCD40-Fc surface |
| | ka | kd | KD | (Avidity) |
| Ligand | (1/Ms) | (1/s) | (nM) | KDₐₚₚₐᵣₑₙₜ (nM) |
| 3h56-269-IgG4.1 | 8.5E+03 | 9.7E−05 | 11.5 | <1 |
| 3h56-269-CT | 1.6E+04 | 1.3E−04 | 8.0 | <1 |
| | 1.6E+04 | 1.2E−04 | 7.3 | <1 |
| 3h56-269-CT | 1.6E+04 | 1.3E−04 | 7.7 | <1 |
| (UCOE-CHO)* | 1.9E+04 | 1.4E−04 | 7.0 | <1 |
| 3h56-269-IgG1.1f | 9.6E+03 | 1.0E−04 | 10.8 | <1 |
| 3h56-269-IgG1.3f | 9.9E+03 | 9.1E−05 | 9.2 | <1 |
| | 1.1E+04 | 1.1E−04 | 9.8 | <1 |
| 3h56-269-IgG1- | 1.1E+04 | 9.9E−05 | 9.0 | <1 |
| D265A | | | | |

*3h-56-269-CT expressed and purified from UCOE-CHO cells.

The FcgR binding properties of the dAb-Fc molecules and the various control monoclonal 1F4 antibodies were characterized by SPR. The first assay involved binding of 1 μM or 10 μM dAb-Fcs or a human-IgG1f antibody control (1F4-IgG1f) to anti-His Fab captured FcgR-His surfaces. These data are shown in Table 10.

TABLE 10
% Rmax data for 1 μM or 10 μM dAb-Fcs or 1F4-IgG1f antibody
control binding to anti-His Fab captured hFcgR-His proteins.

| | | Anti-His Fab captured FcgR | | | | | |
|---|---|---|---|---|---|---|---|
| | | High | | | | | |
| | | affinity | Low Affinity FcgR | | | | |
| | Conc | FcgR | hCD32a- | hCD32a- | | hCD16a- | hCD16b- |
| Sample | (μM) | hCD64 | H131 | R131 | hCD32b | V158 | NA2 |
| 1F4-IgG1f | 1 | 65% | 31% | 19% | 5% | 31% | 13% |
| 3h56-269-IgG4.1 | 1 | 68% | 27% | 30% | 20% | 6% | 1% |
| 3h56-269-IgG1- | 1 | 68% | 7% | 2% | 0% | 1% | 0% |
| D265A | | | | | | | |
| 3h56-269-IgG1.1f | 1 | 11% | 1% | 10% | 3% | 0% | 0% |
| 3h56-269-IgG1.3f | 1 | 12% | 1% | 8% | 3% | 1% | 0% |
| 3h56-269-CT | 1 | 72% | 0% | 1% | 0% | 1% | 0% |
| 1F4-IgG1f | 10 | 65% | 62% | 51% | 24% | 52% | 36% |
| 3h56-269-IgG4.1 | 10 | 69% | 65% | 66% | 57% | 27% | 9% |
| 3h56-269-IgG1- | 10 | 69% | 33% | 17% | 2% | 2% | −1% |
| D265A | | | | | | | |
| 3h56-269-IgG1.1f | 10 | 39% | 6% | 43% | 21% | 3% | 2% |
| 3h56-269-IgG1.3f | 10 | 39% | 6% | 37% | 19% | 5% | 4% |
| 3h56-269-CT | 10 | 70% | 2% | 10% | 3% | 6% | −1% |

In another assay. FcgR analytes (at 1 μM or 10 μM) were tested for binding to protein A-captured dAb-Fc surfaces (data shown in Table 11) and for binding to antibody surfaces (data shown in Table 12).

TABLE 11
% RMax data for 1 μM or 10 μM FcgRs binding
to protein A-captured dAb-Fc proteins.

| | | Protein A-captured dAb-Fc protein | | | | |
|---|---|---|---|---|---|---|
| | | | 3h56-269- | | | |
| | Conc | 3h56-269- | IgG1- | 3h56-269- | 3h56-269- | 3h56- |
| Sample | (μM) | IgG4.1 | D265A | IgG1.1f | IgG1.3f | 269-CT |
| hCD64 | 1 | 99% | 41% | 1% | 2% | 80% |
| hCD32a-H131 | 1 | 29% | 3% | 0% | 1% | 1% |
| hCD32a-R131 | 1 | 31% | 1% | 4% | 5% | 1% |
| hCD32b | 1 | 19% | 0% | 1% | 2% | 1% |
| hCD16a-V158 | 1 | 12% | 0% | 0% | 1% | 1% |
| hCD16B-NA2 | 1 | 2% | 0% | 0% | 0% | 0% |
| hCD64 | 10 | 119% | 85% | 3% | 7% | 114% |
| hCD32a-H131 | 10 | 70% | 18% | 4% | 6% | 7% |
| hCD32a-R131 | 10 | 71% | 4% | 18% | 26% | 7% |
| hCD32b | 10 | 59% | 1% | 9% | 12% | 4% |
| hCD16a-V158 | 10 | 47% | 2% | 2% | 6% | 7% |
| hCD16B-NA2 | 10 | 13% | 0% | 1% | 3% | 1% |

TABLE 12
% Rmax data for 1 μM or 10 μM FcgRs binding to protein A captured antibodies.

| | | Protein A-captured antibodies | | | | | |
|---|---|---|---|---|---|---|---|
| | Conc | 1F4- | 1F4- | 1F4- | 1F4- | 1F4- | |
| Sample | (μM) | IgG1f | IgG4.1 | D265A | IgG1.1f | IgG1.3f | 1F4-CT |
| hCD64 | 1 | 138% | 126% | 96% | 8% | 5% | 120% |
| hCD32a-H131 | 1 | 62% | 29% | 7% | 2% | 1% | 2% |
| hCD32a-R131 | 1 | 48% | 33% | 3% | 5% | 3% | 2% |
| hCD32b | 1 | 11% | 17% | 1% | 1% | 1% | 1% |
| hCD16a-V158 | 1 | 97% | 15% | 1% | 3% | 2% | 2% |
| hCD16B-NA2 | 1 | 33% | 4% | 1% | 4% | 3% | 0% |
| hCD64 | 10 | 155% | 139% | 131% | 17% | 14% | 131% |
| hCD32a-H131 | 10 | 99% | 79% | 38% | 7% | 6% | 11% |
| hCD32a-R131 | 10 | 101% | 87% | 17% | 28% | 24% | 13% |
| hCD32b | 10 | 55% | 68% | 4% | 11% | 12% | 8% |
| hCD16a-V158 | 10 | 125% | 59% | 2% | 5% | 7% | 11% |
| hCD16B-NA2 | 10 | 81% | 16% | −2% | 4% | 6% | 1% |

Based on the binding responses or lack thereof in these experiments, a subset of the higher affinity dAb-Fc/FcgR or Ab/FcgR interactions with strongest binding responses were selected for kinetic/affinity characterization using analyte titrations (FcgR analytes binding to protein A captured antibodies or dAb-Fcs). These data are presented in Table 13.

TABLE 13
KD values (in nM) for purified FcgR analytes binding
to protein A captured antibodies or dAb-Fcs.

| | | hCD32a- | hCD32a- | | hCD16a- | hCD16B- |
|---|---|---|---|---|---|---|
| Sample | hCD64 | H131 | R131 | hCD32b | V158 | NA2 |
| 1F4-IgG1f | 0.2 | 920 | 1400 | >5000 | 430 | 4800 |
| 1F4-IgG4.1 | 0.58 | 3700 | 2400 | 3100 | >5000 | >5000 |
| 3h56-269-IgG4.1 | 2.8 | >5000 | 2200 | >5000 | >5000 | >5000 |
| 3h56-269-IgG1-D265A | 62 | | | | | |
| 3h56-269-IgG1.1f | >5000 | | | | | |
| 3h56-269-IgG1.3f | >5000 | >5000 | >5000 | >5000 | >5000 | >5000 |
| 3h56-269-CT | 4.6 | >5000 | >5000 | >5000 | >5000 | >5000 |

Collectively, these FcgR binding SPR data show that the IgG1f and IgG4.1 isotype molecules have significantly higher FcgR affinity across all FcgRs as compared to the modified Fc variant IgG1-D265A, IgG1.1f, IgG1.3f, or CT molecules. Of the modified Fc variants, the hCD64 binding affinity was the strongest for 3h56-269-CT (KD=4.6 nM), weaker for 3h56-269-IgG1-D265A (KD=62 nM), and the weakest for 3h56-269-IgG1.1f and 3h56-269-IgG1.3f, for which affinity was too weak to quantitate under the conditions tested (KD>5 UM, which is half of the highest analyte concentration tested). All of the other FcgR interactions (hCD32a-H131, hCD32a-R131, hCD32b, hCD16a-V158, hCD16b-NA2) for the IgG1-D265A, IgG1.1f, IgG1.3f and CT variants were also too weak to obtain reliable KD values (KD>5 μM). However, differences in the relative binding responses can be observed in the % Rmax data. For example, the IgG1-D265A variant has stronger binding response for hCD32a-H131 as compared to the IgG1.1f, IgG1.3f or CT variants (Table 11). In contrast, the IgG1.1f and IgG1.3f variants have stronger binding responses for hCD32a-R131 as compared to the IgG1-D265A and CT variants (Table 11).

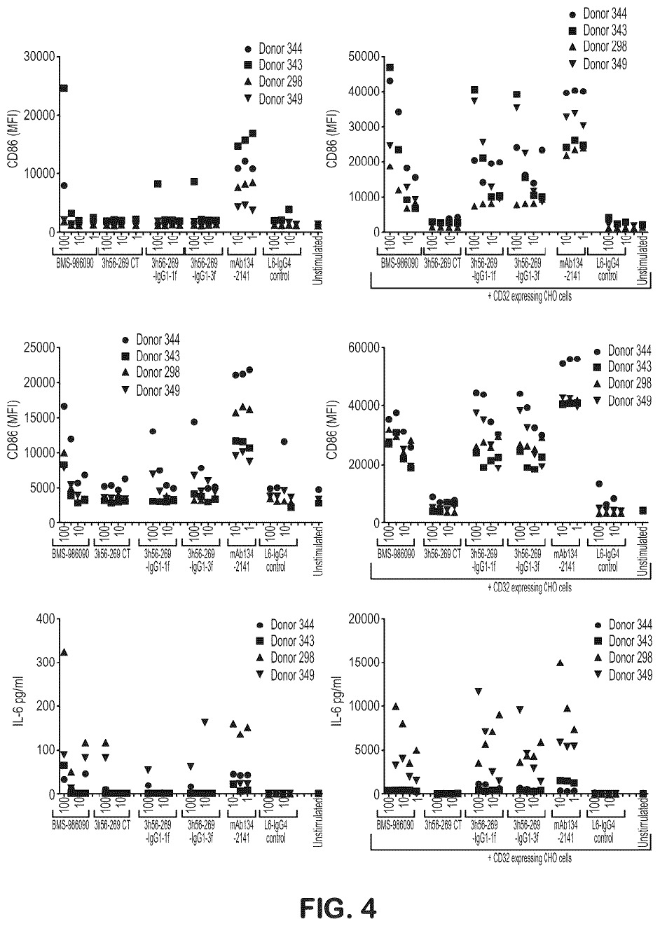

The dAb-Fc molecules were tested in the iDC assay (described in Example 1) with and without CD32-over-expressing CHO cell cross-linking. These data are shown in FIG. 4. The 3h-59-269-CT molecule did not produce iDC activation above controls levels at concentrations up to 100 μg/ml (left hand panels of FIG. 4), even when cross-linked by use of CD32-expressing CHO cells (right hand panels of FIG. 4). However Fc-fusions of the domain antibody with changes designed to minimize FcgR binding (3h-59-269-IgG1.1f and 3h-59-269-IgG1.3f) exhibited reduced, but still measurable, iDC activation as measured by upregulation in CD54 (also called ICAM 1) and CD86 expression and increased cytokine release from at least 1 of the 4 donors tested at the highest concentration tested of 100 μg/ml. Inclusion of CD32-expressing CHO cell to introduce cross-linking led to robust iDC activation, as measured by CD86 and CD54 expression upregulation, in all 4 donors. These data demonstrate the FcgR dependence of the iDC activation observed.

### Example 3: Developability Assessment for dAb-Fc Proteins

Bringing a protein therapeutic to market requires the molecule to have suitable physical and chemical properties for development, commonly referred to as Chemistry Manufacturing and Control (CMC). The physical and chemical properties of the molecule, including stability, solubility, and homogeneity, are also collectively referred to as “developability”. Many techniques and assays have been developed to assess the developability of a protein therapeutic candidate molecule, some of which include differential scanning calorimetry (DSC), imaged capillary isoelectric focusing (icIEF), mass spectrometry (MS or mass spec), and accelerated stability studies.

The developability of various dAb-Fc proteins was assessed by DSC, icIEF and mass spectrometry. Materials and methods are described below.

Differential Scanning calorimetry: DSC experiments were performed on a MicroCal VP-Capillary DSC instrument (Malvern Instruments, Malvern, UK) in 10 mM NaPO4, 130 mM NaCl pH 7.1. Samples of 1 mg/ml dAb-Fc or antibody were tested using a scan range of 10-110° C. and a scan rate of 90° C./hr. Data were analyzed using MicroCal-Origin 7.0 software.

Imaged Capillary Isoelectric Focusing: icIEF experiments were performed on a ProteinSimple iCE3™ System (ProteinSimple, San Jose, CA). For these studies the dAb-Fc or antibody samples, typically at 2 mg/ml concentration, were mixed with a carrier ampholyte mixture consisting of 2 M urea, 0.35% methylcellulose, 1% Pharmalyte 5-8, 3% Pharmalyte 8-10.5, and pI markers 5.85 and 10.10, to a final protein concentration of 0.20 mg/mL, and analyzed using a pre-focusing time of 1 min at 1.5 kV and a focusing time of 10 min at 3 kV.

Mass Spectrometry: For mass spectrometry (mass spec) analysis, samples were reduced using 100 mM DTT, and N-deglycosylation was performed with peptide: N-Glycosidase (FPNGaseF). Liquid chromatography-mass spectrometry (LC/MS) instrumentation used was a Waters Synapt® G2 (Waters Corporation, Milford, MA) with a Waters Acquity® UPLC (ultra-performance liquid chromatography). The UPLC column was a Waters Acquity® BEH (ethylene bridged hybrid particle) C4 (2.1×150 mm, 300 Å, 1.7 um particle). The gradient was 10% to 38% (Mobile phase B) in 10 min at 200 μL/min flow rate. Mobile phase A was 0.1% formic acid in water. Mobile phase B was 0.1% formic acid in acetonitrile. Column temperature was 60° C. Data analysis was performed manually with the aid of Waters MassLynx™ software; spectral deconvolution was performed with the MaxEnt1 algorithm.

Accelerated Stability Studies: Accelerated stability studies were conducted by first extensively dialyzing dAb-Fc molecules in target formulation buffers at 4° C. Samples were recovered and concentrated using Amicon® Ultra Centrifugal Filter Units (Merck KgaA, Germany) and prepared at different target concentrations in dialysis buffer. These samples were incubated at various temperatures, typically 4° C., 25° C., 32° C., and/or 40° C. for several weeks, with aliquots removed and analyzed by analytical size exclusion chromatography. Analytical size exclusion chromatography was conducted on an Agilent 1260 HPLC, using a Shodex™ K403-4F column (Showa Denko America, Inc., New York, NY) in a mobile phase of 100 mM Sodium Phosphate, 150 mM Sodium Chloride, pH 7.3, flow rate of 0.3 ml/min.

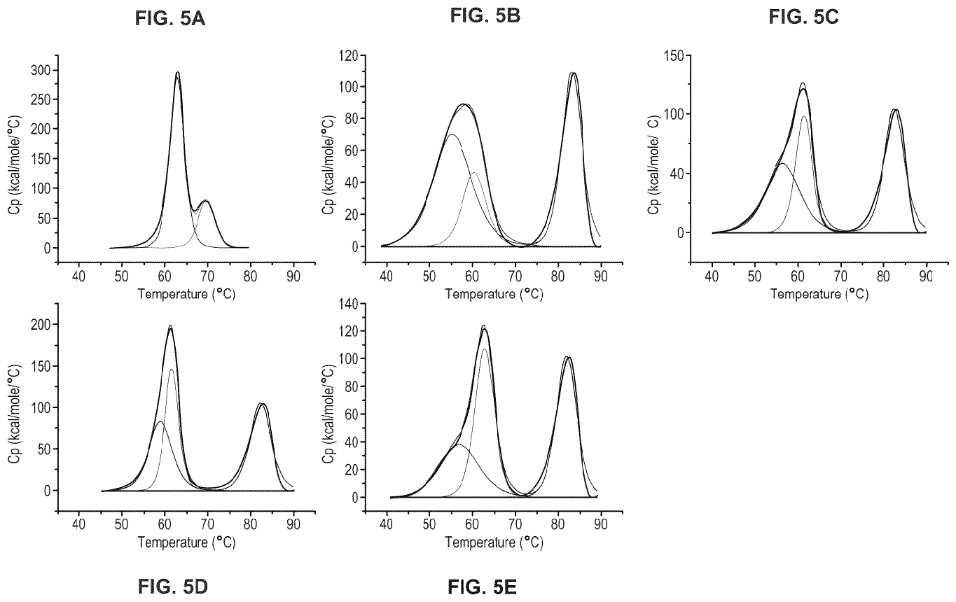

Results—Differential scanning calorimetry: DSC can be used to measure the thermal stability of a protein. The DSC data for 3h56-269 dAb formatted with different Fc domains is shown in FIGS. 5A-5E. The best fit Tm values are summarized in Table 14.

TABLE 14
Thermal melting temperature (Tm) values for
dAb-Fc molecules as determined by DSC.

| | Tm dAb and CH2 domains | | Tm CH3 |
|---|---|---|---|
| Sample | Tm1 (° C.) | Tm2 (° C.) | domain (° C.) |
| 3h56-269-IgG4.1 | 62.8 | | 69.6 |
| 3h56-269-CT | 55.4 | 60.4 | 83.2 |
| 3h56-269-IgG1.1f | 59.0 | 61.6 | 82.3 |
| 3h56-269-IgG1.3f | 57.0 | 62.8 | 81.9 |
| 3h56-269-IgG1-D265A | 56.4 | 61.4 | 82.4 |

Based on the characteristic thermal denaturation profiles for IgG Fc domains, the Fc CH3 domain transition for 3h56-269-IgG4.1 was assigned as the transition with midpoint (Tm) value of 69.6° C.; and the Fc CH3 domain of the various IgG1 molecules was assigned as the transition with Tm near ˜82-83° C. The denaturation of the dAb domain and CH2 domain for the dAb-Fcs were assigned to the transition(s) below 65° C., which differ between the different constructs, both in the onset of thermal denaturation (Tₒₙₛₑₜ), the shape of the unfolding transition, and the best fit Tm values. For example, the thermal transition for the dAb and CH2 domains of 3h56-269-IgG4.1 appears as a single overlapping or cooperative transition, with Tm value of 62.8° C. The denaturation profile for the dAb and CH2 domains of 3h56-269-IgG1-D265A, 3h56-269-IgG1.1f and 3h56-269-IgG1.3f are all consistent with a more asymmetrical transition, which was best described by two transitions having Tm values between ˜56-63° C. 3h56-269-CT had the lowest Tₒₙₛₑₜ, beginning to unfold near 40° C., with a broad thermal transition and the lowest fitted Tm values of Tm1=55.4° C. and Tm2=60.4° C.

Results—Imaged capillary isoelectric focusing (icIEF): Imaged capillary isoelectric focusing (icIEF) can be used to characterize sample homogeneity or heterogeneity. The ability to generate a homogeneous product is another important developability criterion. Consequently, during the discovery and optimization of a novel protein therapeutic, various analytical methods are utilized to characterize and quantitate sample heterogeneities, and to select for the most homogeneous molecules.

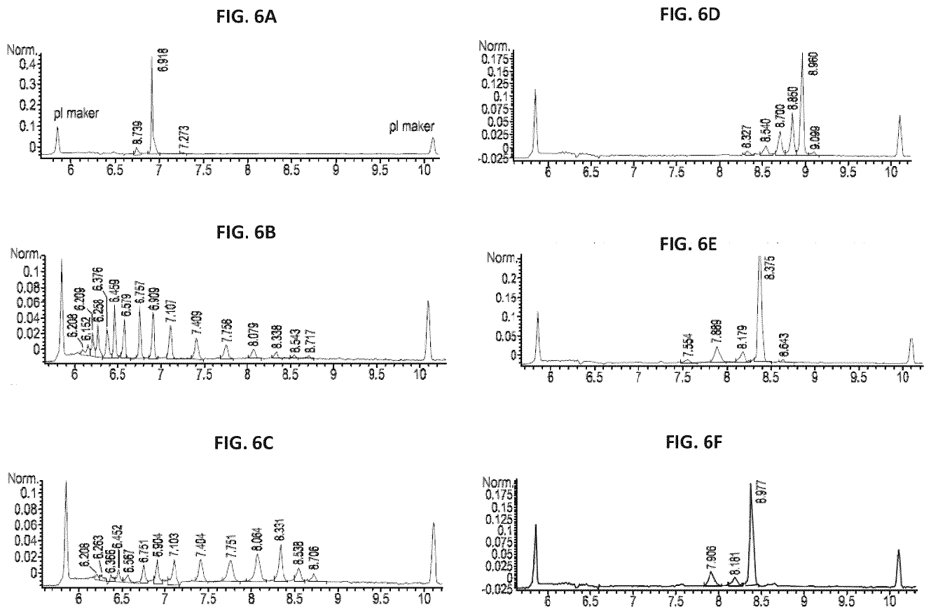

The charge profiles for dAb-Fc molecules were characterized by icIEF. The data are shown in FIGS. 6A-6F. The icIEF profiles for 3h56-269-IgG4.1 (FIG. 6A), 3h56-269-IgG1.1f (FIG. 6E) and 3h56-269-IgG1.3f (FIG. 6F) are all relatively simple, each consisting of a distinct main peak with area of 69-86%, and between two and four charge variants in lower abundance. This icIEF profile is similar to the typical profile obtained for an antibody. The main peak for 3h56-269-IgG1-D265A (FIG. 6D) is somewhat lower abundance (49%) with a corresponding higher level of acidic variants with at least six detectable species. In contrast, the profile for 3h56-269-CT (FIG. 6B) is highly heterogeneous, consisting of at least 16 different species and no clear main peak. The icIEF profile for 3h56-269-CT expressed in a different cell line (UCOE-CHO) was equally heterogeneous (FIG. 6C), although the distribution of the charge variants was considerably different from the HEK293-expressed material.

### Results—Mass Spectrometry: Typical Glycosylation on the Fc Domain of IgG or

Fc-containing proteins is a mixture of GOF, GIF and some G2F species. Other glycoforms, such as sialylated or non-fucosylated forms, are generally found in much lower abundance or at undetectable levels.

To characterize the glycosylation profiles of the dAb-Fc proteins, and to compare the dAb-Fc proteins to control antibodies with similar Fc mutations, mass spectrometry experiments were conducted. The data are shown in Table 15.

TABLE 15
Detectable glycoforms in dAb-Fc and antibody
molecules as determined by mass spectrometry.

| Sample | G0F | G1F | G2F | G2FS1 | G2FS2 |
|---|---|---|---|---|---|
| 3h56-269-IgG4.1 | 67% | 29% | 4% | | |
| 3h56-269-IgG1.1f | 32% | 58% | 9% | | |
| 3h56-269-IgG1.3f | 42% | 55% | 3% | | |
| 3h56-269-IgG1-D265A | 4% | 37% | 43% | 13% | 2% |
| 1F4-IgG1f | 68% | 32% | | | |
| 1F4-IgG1.3f | 26% | 64% | 10% | | |
| 1F4-D265A | 27% | 40% | 27% | 4% | 2% |

The mass spectrometry data for the control antibodies 1F4-IgG1f and 1F4-IgG1.3f, as well as for dAb-Fc antibodies 3h56-269-IgG4.1, 3h56-269-IgG1.1f, 3h56-269-IgG1.3f, showed that these proteins consist of a typical mixture of GOF, GIF glycoforms, with a lower abundance of G2F species.

Both the dAb-Fc and antibody molecules containing the D265A mutation in the Fc domain also contained a mixture of GOF, GIF, and G2F species, but in addition they had higher levels of sialylated glycoforms. All of these D265A molecules could be deglycosylated using standard PNGase enzyme treatment protocols; this data is consistent with the glycan of the D265A molecules being N-linked and occupying the common Asn297 residue in the Fc domain.

In contrast, the mass spectrometry data for 3h56-269-CT expressed in either HEK293 or UCOE-CHO cells, or the control 1F4-CT antibody, revealed that these proteins were very heterogeneous, with evidence for numerous different complex glycosylated species including highly sialylated species. The data for 3h56-269-CT is presented in Table 16.

TABLE 16
Detectable glycoforms in 3h56-269-CT molecules
as determined by mass spectrometry.

| | | | 3h56- |
|---|---|---|---|
| | | | 269-CT |
| | Mass | 3h56- | (UCOE- |
| Glycan Forms | (Da) | 269-CT | CHO) |
| without glycosylation | 39000 | | 2.1% |
| O-(Hex)1 (HexNAc)1 (NeuAc)1 | 39655 | | 1.9% |
| unassigned | 40607 | 2.2% | |
| N-G0F; O-(HexNAc)1 | 40648 | | |
| +C-terminal Lys with N-G0F; O-(HexNAc)1 | 40770 | 2.6% | 16.0% |
| unassigned | 40810 | 2.7% | |
| N-G0F; O-(HexNAc)2 | 40851 | | |
| +C-terminal Lys with N-G0F; O-(HexNAc)2 | 40973 | 1.9% | 6.0% |
| unassigned | 41056 | | |
| unassigned | 41102 | 2.2% | |
| N-G0F; O-(HexNAc)4 | 41261 | 3.2% | 9.9% |
| unassigned | 41304 | | |
| +C-terminal Lys with N-G0F; O-(Hex)1 | 41427 | | 28.3% |
| (HexNAc)2 (NeuAc)1 | | | |
| unassigned | 41467 | | |
| unassigned | 41508 | | |
| unassigned | 41554 | 5.4% | |
| N-G0F; O-(Hex)1(HexNAc)1 (NeuAc)1 | | | |
| N-G0F; O-(Hex)1 (HexNAc)4 (NeuAc)1 | 41714 | 5.5% | 7.8% |
| N-G0F; O-(Hex)2 (HexNAc)2 (NeuAc)2 | 41761 | 2.1% | |
| N-G1F; O-(Hex)1(HexNAc)1 (NeuAc)1 | | | |
| N-G1F; O-(Hex)2 (HexNAc)2 (NeuAc)2 | 41921 | 3.6% | 2.9% |
| unassigned | 41963 | | |
| unassigned | 42009 | 2.5% | 3.7% |
| N-G2F; O-(Hex)2 (HexNAc)2 (NeuAc)2 | 42085 | 3.1% | 10.1% |
| N-G0F; O-(Hex)2 (HexNAc)4 (NeuAc)2 | 42161 | | |
| unassigned | 42211 | 3.4% | |
| N-G2F; O-(Hex)2 (HexNAc)2 (NeuAc)3 | 42375 | 3.7% | 4.7% |
| unassigned | 42414 | 2.3% | |
| unassigned | 42456 | | |
| unassigned | 42502 | 3.6% | |
| N-G0F; O-(Hex)3 (HexNAc)4 (NeuAc)3 | 42614 | 4.4% | |
| unassigned | 42646 | 2.0% | |
| N-G2F; O-(Hex)2 (HexNAc)2 (NeuAc)4 | 42665 | 6.4% | 4.0% |
| unassigned | 42706 | 2.1% | |
| unassigned | 42721 | 2.6% | |
| unassigned | 42738 | 4.3% | |
| unassigned | 42782 | | |
| N-G2F; O-(Hex)2 (HexNAc)3 (NeuAc)4 | 42867 | 4.3% | |
| N-G0F; O-(Hex)3(HexNAc)3 (NeuAc)3 | | | |
| N-G0F; O-(Hex)3(HexNAc)4 (NeuAc)3 | | | |
| N-G0F; O-(Hex)3 (HexNAc)4 (NeuAc)4 | 42905 | | |
| N-G0F; O-(Hex)4(HexNAc)4 (NeuAc)3 | | | |
| N-G0F; O-(Hex)4(HexNAc)4 (NeuAc)4 | | | |
| N-G2F; O-(Hex)3 (HexNAc)3 (NeuAc)4 | 43030 | 5.3% | |
| N-G0F; O-(Hex)4 (HexNAc)4 (NeuAc)4 or | 43067 | 2.3% | |
| N-G1F; O-(Hex)3 (HexNAc)4(NeuAc)4 | | | |
| N-G1F; O-(Hex)4 (HexNAc)4 (NeuAc)4 or | 43230 | | |
| N-G2F; O-(Hex)3 (HexNAc)4(NeuAc)4 | | | |
| N-G2F; O-(Hex)3 (HexNAc)3 (NeuAc)5 | 43320 | 3.5% | |
| unassigned | 43396 | 2.1% | |
| N-G0F; O-(Hex)4 (HexNAc)4 (NeuAc)5 | 43359 | | |
| N-G1F; O-(Hex)4(HexNAc)4 (NeuAc)4 | | | |
| N-G1F; O-(Hex)4 (HexNAc)4 (NeuAc)5 | 43530 | | |
| N-G2F; O-(Hex)4(HexNAc)4 (NeuAc)4 | | | |
| N-G2F; O-(Hex)4 (HexNAc)4 (NeuAc)5 | 43691 | 2.2% | 2.6% |
| unassigned | 43890 | 2.4% | |
| N-G2F; O-(Hex)4 (HexNAc)4 (NeuAc)6 | 43974 | | |
| unassigned | 44052 | 2.9% | |
| unassigned | 44343 | 3.3% | |

The mass spec data for the control 1F4-CT antibody is shown in Table 17.

TABLE 17
Detectable glycoforms in 1F4-CT as
determined by mass spectrometry.

| | | Abun- | |
|---|---|---|---|
| ID# | Mass | dance | Proposed Assignment |
| 1 | 50480 | 2% | N-G0F |
| 2 | 50640 | 2% | N-G1F |
| 3 | 50682 | 6% | N-G0F; O-(HexNAc) |
| 4 | 50801 | 1% | N-G2F or N-G1F; O-(Hex) |
| 5 | 50844 | 4% | N-G0FN; O-(Hex) or N-G1F; O-(HexNAc) |
| 6 | 50885 | 1% | N-G0FN; O-(HexNAc) |
| 7 | 51006 | 2% | N-G2FN |
| 8 | 51048 | 1% | N-G1FN; O-(HexNAc) |
| 9 | 51136 | 10% | N-G0F; O-(Hex)(HexNAc)(NeuAc) or N- |
| | | | G1F; O-(HexNAc)(NeuAc) |
| 10 | 51297 | 11% | N-G1F; O-(Hex)(HexNAc)(NeuAc) or N- |
| | | | G2F; O-(HexNAc)(NeuAc) |
| 11 | 51339 | 4% | N-G0FN; O-(Hex)(HexNAc)(NeuAc) or |
| | | | N-G1FN; O- |
| | | | (HexNAc)(NeuAc) |
| 12 | 51426 | 4% | N-G1FS; O-(HexNAc)(NeuAc) |
| 13 | 51459 | 3% | N-G2F; O-(Hex)(HexNAc)(NeuAc) |
| 14 | 51501 | 3% | N-G1FN; O-(Hex)(HexNAc)(NeuAc) |
| 15 | 51589 | 15% | N-G1F; O-(Hex)(HexNAc)(NeuAc)2 |
| 16 | 51751 | 18% | N-G2F; O-(Hex)(HexNAc)(NeuAc)2 |
| 17 | 51792 | 9% | N-G1FN; O-(Hex)(HexNAc)(NeuAc)2 |
| 18 | 51954 | 3% | N-G2FN; O-(Hex)(HexNAc)(NeuAc)2 |
| 19 | 52042 | 2% | N-G2FS; O-(Hex)(HexNAc)(NeuAc)2 |

In addition, the 3h56-269-CT and 1F4-CT molecules could not be effectively deglycosylated by treatment with PNGase; these results suggest that at least some of the complex glycan was O-linked, on Ser or Thr residues. These data are consistent with the known glycosylation of abatacept which contains the same modified IgG1 Fc domain containing C220S, C226S, C229S, and P238S mutations. In abatacept, it has been shown that these introduced Ser mutations are sites for O-linked glycosylation in the hinge region, which are heterogeneously glycosylated and high in sialic acid species.

Results—Accelerated Stability Studies: 3h56-269-CT was selected for additional studies including additional developability assessment, because 3h56-269-CT was the only dAb-Fc molecule that demonstrated no response in the iDC assay in either the absence or presence of CD32 over-expressing CHO cell cross-linking. In particular, stability studies were conducted under accelerated stress conditions of 32° C. and 40° C., as well as lower temperatures of 4° C. and 25° C. The formulation buffer for these studies (20 mM potassium phosphate, 250 mM sucrose, 50 μM DTPA and 0.05% PS80, pH7.0) was selected based on screening the thermal stability of the molecule using the UNit platform (Unchained Labs, Woburn, MA), to identify conditions which gave favorable thermal stability (Tm) and onset of aggregation (Tagg). The purified 3h56-269-CT protein was exchanged into this formulation buffer by dialysis, then concentrated and prepared at final concentration of either 50 mg/ml or 150 mg/ml, and incubated at various temperatures for 4 weeks. To evaluate the physical stability of the protein, aliquots were removed at time zero (t0), 1 week (1 w) and 4 weeks (4 w) after initiation of the different temperature incubations. The samples were analyzed by analytical size exclusion chromatography (aSEC) to determine the levels of monomeric protein, high molecular weight aggregate (HMW), and low molecular weight species (LMW) species. The HMW data are shown in Table 18.

TABLE 18
Percentage of high molecular weight (HMW) species as determined
by analytical size exclusion chromatography in samples of 3h56-
269-CT incubated at different temperatures for 4 weeks.

| | | |
|---|---|---|
| Conc | Temp | % HMW (aSEC) |

| (mg/ml) | (° C.) | t0 | 1 w | 4 w |
|---|---|---|---|---|
| 50 | 32 | 1.0% | 5.5% | 10.6% |
| 50 | 40 | 1.0% | 16.8% | 23.1% |
| 150 | 4 | 1.3% | 2.0% | 3.1% |
| 150 | 25 | 1.3% | 5.7% | 8.0% |
| 150 | 32 | 1.3% | 9.1% | 16.4% |
| 150 | 40 | 1.3% | 22.4% | 28.9% |

The aSEC data showed high levels of HMW formation for 3h56-269-CT, particularly at higher protein concentration and higher temperatures.

### Example 4: Variant Fc Domains

As shown in Examples 1 and 2, the 3h56-269-CT molecule advantageously was found to have favorably weak FcgR binding, particularly towards the low affinity FcgRs (hCD32a, hCD32b, hCD16a, hCD16b), and also demonstrated a lack of response in the iDC assay including with CD32 over-expressing CHO cell cross-linking. However, as shown in Example 3, biophysical characterization of 3h56-269-CT showed that the molecule has low thermal stability, high heterogeneity, and poor physical stability. Consequently, an effort was initiated to improve the 3h56-269-CT molecule was performed, with the goal of reducing or eliminating O-linked glycan, reducing or eliminating sialic acid content, reducing heterogeneity, and improving thermal and physical stability, while maintaining the favorably weak FcgR binding and lack of signal in the iDC assay.

In order to try to improve the biophysical characteristics of 3h56-269-CT, a series of mutant dAb-Fc molecules was designed to attempt to understand the contribution of the individual C220S, C226S, C229S and P238S mutations to the properties of 3h56-269-CT, and to try to decouple the undesirable developability challenges from the desired weak FcgR binding and lack of Fc-mediated signaling. The mutation strategy involved design of several variants at positions 220, 226, 229, and 238 (Kabat numbering). The following variants were designed:
a) a set of single and combination Ser mutants at positions 220, 226, 229 and 238 to test the individual and combined effects of these mutations. See SEQ ID NOs: 88-96 in Table 19. The underlined sequence is the anti-CD40 single variable domain.

TABLE 19

| SEQ | | | |
|---|---|---|---|
| ID | | | |
| NO: | Long name | Short ID | Sequence |
| 88 | 3h56-269-CTf | 3h56-269- | EVQLLESGGGLVQPGGSLRLSCAASGFTFRDYEMWWVRQAPGKG |
| | | CTf | LERVSAINPQGTRTYYADSVKGRFTISRDNSKNTLYLQMNSLRAED |
| | | | TAVYYCAKLPFRFSDRGQGTLVTVSSASTEPKSSDKTHTSPPSPAPE |
| | | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 89 | 3h56-269- | 3h56-269- | EVQLLESGGGLVQPGGSLRLSCAASGFTFRDYEMWWVRQAPGKG |
| | IgG1a | IgG1a- | LERVSAINPQGTRTYYADSVKGRFTISRDNSKNTLYLQMNSLRAED |
| | | | TAVYYCAKLPFRFSDRGQGTLVTVSSASTEPKSCDKTHTCPPCPAPE |
| | | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 90 | 3h56-269- | 3h56-269- | EVQLLESGGGLVQPGGSLRLSCAASGFTFRDYEMWWVRQAPGKG |
| | IgG1a-C220S | IgG1a-SCCP | LERVSAINPQGTRTYYADSVKGRFTISRDNSKNTLYLQMNSLRAED |
| | | | TAVYYCAKLPFRFSDRGQGTLVTVSSASTEPKSSDKTHTCPPCPAPE |
| | | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 91 | 3h56-269- | 3h56-269- | EVQLLESGGGLVQPGGSLRLSCAASGFTFRDYEMWWVRQAPGKG |
| | IgG1a- | IgG1a-SSCP | LERVSAINPQGTRTYYADSVKGRFTISRDNSKNTLYLQMNSLRAED |
| | C220S, C226S | | TAVYYCAKLPFRFSDRGQGTLVTVSSASTEPKSSDKTHTSPPCPAPE |
| | | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 92 | 3h56-269 | 3h56-269- | EVQLLESGGGLVQPGGSLRLSCAASGFTFRDYEMWWVRQAPGKG |
| | IgG1a- | IgG1a-SCSP | LERVSAINPQGTRTYYADSVKGRFTISRDNSKNTLYLQMNSLRAED |
| | C220S, C229S | | TAVYYCAKLPFRFSDRGQGTLVTVSSASTEPKSSDKTHTCPPSPAPE |
| | | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 93 | 3h56-269 | 3h56-269- | EVQLLESGGGLVQPGGSLRLSCAASGFTFRDYEMWWVRQAPGKG |
| | IgG1a- | IgG1a-SCCS | LERVSAINPQGTRTYYADSVKGRFTISRDNSKNTLYLQMNSLRAED |
| | C220S, P238S | | TAVYYCAKLPFRFSDRGQGTLVTVSSASTEPKSSDKTHTCPPCPAPE |
| | | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 94 | 3h56-269- | 3h56-269- | EVQLLESGGGLVQPGGSLRLSCAASGFTFRDYEMWWVRQAPGKG |
| | IgG1a- | IgG1a-SSSP | LERVSAINPQGTRTYYADSVKGRFTISRDNSKNTLYLQMNSLRAED |
| | C220S, C226S, | | TAVYYCAKLPFRFSDRGQGTLVTVSSASTEPKSSDKTHTSPPSPAPE |
| | C229S | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 95 | 3h56-269- | 3h56-269- | EVQLLESGGGLVQPGGSLRLSCAASGFTFRDYEMWWVRQAPGKG |
| | IgG1a- | IgG1a-SSCS | LERVSAINPQGTRTYYADSVKGRFTISRDNSKNTLYLQMNSLRAED |
| | C220S, C226S, | | TAVYYCAKLPFRFSDRGQGTLVTVSSASTEPKSSDKTHTSPPCPAPE |
| | P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 96 | 3h56-269- | 3h56-269- | EVQLLESGGGLVQPGGSLRLSCAASGFTFRDYEMWWVRQAPGKG |
| | IgG1a- | IgG1a-SCSS | LERVSAINPQGTRTYYADSVKGRFTISRDNSKNTLYLQMNSLRAED |
| | C220S, C229S, | | TAVYYCAKLPFRFSDRGQGTLVTVSSASTEPKSSDKTHTCPPSPAPE |
| | P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |

b) a set of single and combination Ala or combination Ala and Ser mutants at positions 226, 229 and 238, to identify the primary sites of O-linked glycosylation and impact on molecule properties. Like Ser mutations, Ala mutations at C220, C226 and C229 are expected to prevent disulfide bond formation. However, unlike Ser, Ala residues are not sites for O-linked glycosylation. See SEQ ID NOs: 97-109 in Table 20.

TABLE 20

| SEQ | | | |
|---|---|---|---|
| ID | | | |
| NO: | Long name | Short ID | Sequence |
| 97 | 3h56-269- | 3h56-269- | EVQLLESGGGLVQPGGSLRLSCAASGFTFRDYEMWWVRQAPGKG |
| | IgG1a- | IgG1a-SACP | LERVSAINPQGTRTYYADSVKGRFTISRDNSKNTLYLQMNSLRAED |
| | C220S, C226A | | TAVYYCAKLPFRFSDRGQGTLVTVSSASTEPKSSDKTHTAPPCPAPE |
| | | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 98 | 3h56-269- | 3h56-269- | EVQLLESGGGLVQPGGSLRLSCAASGFTFRDYEMWWVRQAPGKG |
| | IgG1a- | IgG1a-SCAP | LERVSAINPQGTRTYYADSVKGRFTISRDNSKNTLYLQMNSLRAED |
| | C220S, C229A | | TAVYYCAKLPFRFSDRGQGTLVTVSSASTEPKSSDKTHTCPPAPAPE |
| | | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 99 | 3h56-269- | 3h56-269- | EVQLLESGGGLVQPGGSLRLSCAASGFTFRDYEMWWVRQAPGKG |
| | IgG1a- | IgG1a- | LERVSAINPQGTRTYYADSVKGRFTISRDNSKNTLYLQMNSLRAED |
| | C220S, C226A, | SAAP | TAVYYCAKLPFRFSDRGQGTLVTVSSASTEPKSSDKTHTAPPAPAPE |
| | C229A | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 100 | 3h56-269- | 3h56-269- | EVQLLESGGGLVQPGGSLRLSCAASGFTFRDYEMWWVRQAPGKG |
| | IgG1a- | IgG1a-SAAS | LERVSAINPQGTRTYYADSVKGRFTISRDNSKNTLYLQMNSLRAED |
| | C220S, C226A, | | TAVYYCAKLPFRFSDRGQGTLVTVSSASTEPKSSDKTHTAPPAPAPE |
| | C229A, P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 101 | 3h56-269- | 3h56-269- | EVQLLESGGGLVQPGGSLRLSCAASGFTFRDYEMWWVRQAPGKG |
| | IgG1a- | IgG1a-SACS | LERVSAINPQGTRTYYADSVKGRFTISRDNSKNTLYLQMNSLRAED |
| | C220S, C226A, | | TAVYYCAKLPFRFSDRGQGTLVTVSSASTEPKSSDKTHTAPPCPAPE |
| | P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 102 | 3h56-269 | 3h56-269- | EVQLLESGGGLVQPGGSLRLSCAASGFTFRDYEMWWVRQAPGKG |
| | IgG1a- | IgG1a-SCAS | LERVSAINPQGTRTYYADSVKGRFTISRDNSKNTLYLQMNSLRAED |
| | C220S, C229A, | | TAVYYCAKLPFRFSDRGQGTLVTVSSASTEPKSSDKTHTCPPAPAPE |
| | P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLIVDKSRW |
| | | | QQGNVFSCSVMHEALHNHYTQKSLSLSPGK |
| 103 | 3h56-269- | 3h56-269- | EVQLLESGGGLVQPGGSLRLSCAASGFTFRDYEMWWVRQAPGKG |
| | IgG1a- | IgG1a-SASP | LERVSAINPQGTRTYYADSVKGRFTISRDNSKNTLYLQMNSLRAED |
| | C220S, C226A, | | TAVYYCAKLPFRFSDRGQGTLVTVSSASTEPKSSDKTHTAPPSPAPE |
| | C229S | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 104 | 3h56-269- | 3h56-269- | EVQLLESGGGLVQPGGSLRLSCAASGFTFRDYEMWWVRQAPGKG |
| | IgG1a- | IgG1a-SSAP | LERVSAINPQGTRTYYADSVKGRFTISRDNSKNTLYLQMNSLRAED |
| | C220S, C226S, | | TAVYYCAKLPFRFSDRGQGTLVTVSSASTEPKSSDKTHTSPPAPAPE |
| | C229A | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 105 | 3h56-269- | 3h56-269- | EVQLLESGGGLVQPGGSLRLSCAASGFTFRDYEMWWVRQAPGKG |
| | IgG1a- | IgG1a-SASS | LERVSAINPQGTRTYYADSVKGRFTISRDNSKNTLYLQMNSLRAED |
| | C220S, C226A, | | TAVYYCAKLPFRFSDRGQGTLVTVSSASTEPKSSDKTHTAPPSPAPE |
| | C229S, P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 106 | 3h56-269- | 3h56-269- | EVQLLESGGGLVQPGGSLRLSCAASGFTFRDYEMWWVRQAPGKG |
| | IgG1a- | IgG1a-SSAS | LERVSAINPQGTRTYYADSVKGRFTISRDNSKNTLYLQMNSLRAED |
| | C220S, C226S, | | TAVYYCAKLPFRFSDRGQGTLVTVSSASTEPKSSDKTHTSPPAPAPE |
| | C229A, P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 107 | 3h56-269- | 3h56-269- | EVQLLESGGGLVQPGGSLRLSCAASGFTFRDYEMWWVRQAPGKG |
| | IgG1a- | IgG1a-ASSS | LERVSAINPQGTRTYYADSVKGRFTISRDNSKNTLYLQMNSLRAED |
| | C220A, C226S, | | TAVYYCAKLPFRFSDRGQGTLVTVSSASTEPKSADKTHTSPPSPAPE |
| | C229S, P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 108 | 3h56-269- | 3h56-269- | EVQLLESGGGLVQPGGSLRLSCAASGFTFRDYEMWWVRQAPGKG |
| | IgG1a- | IgG1a- | LERVSAINPQGTRTYYADSVKGRFTISRDNSKNTLYLQMNSLRAED |
| | C220A, C226A, | AASP | TAVYYCAKLPFRFSDRGQGTLVTVSSASTEPKSADKTHTAPPSPAPE |
| | C229S | | LLGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |
| 109 | 3h56-269- | 3h56-269- | EVQLLESGGGLVQPGGSLRLSCAASGFTFRDYEMWWVRQAPGKG |
| | IgG1a- | IgG1a-AASS | LERVSAINPQGTRTYYADSVKGRFTISRDNSKNTLYLQMNSLRAED |
| | C220A, C226A, | | TAVYYCAKLPFRFSDRGQGTLVTVSSASTEPKSADKTHTAPPSPAPE |
| | C229S, P238S | | LLGGSSVFLFPPKPKDTLMISRTPEVTCVVVDVSHEDPEVKFNWYV |
| | | | DGVEVHNAKTKPREEQYNSTYRVVSVLTVLHQDWLNGKEYKCKVS |
| | | | NKALPAPIEKTISKAKGQPREPQVYTLPPSRDELTKNQVSLTCLVKG |
| | | | FYPSDIAVEWESNGQPENNYKTTPPVLDSDGSFFLYSKLTVDKSRW |
| | | | QQGNVFSCSVMHEALHNHYTQKSLSLSPGK |

c) a set of mutants with P238 mutated to lysine (P238K) was designed to test whether FcgR binding affinity could be reduced by a non-conservative positively charged residue at this position in this lower hinge region. See SEQ ID NOs: 110-116 in Table 21.

TABLE 21

| SEQ | | | |
|---|---|---|---|
| ID | | | |
| NO: | Long name | Short ID | Sequence |
| 110 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, P238K | SCCK | VYYCAKLPFRFSDRGQGTLVTVSSASTEPKSSDKTHTCPPCPAPELLG |
| | | | GKSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALP |
| | | | APIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |
| 111 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, C226S, | SSSK | VYYCAKLPFRFSDRGQGTLVTVSSASTEPKSSDKTHTSPPSPAPELLGG |
| | C229S, P238K | | KSVFLFPPKPKDTLMISRTPEVTCVVVDVSHEDPEVKFNWYVDGVEV |
| | | | HNAKTKPREEQYNSTYRVVSVLTVLHQDWLNGKEYKCKVSNKALPA |
| | | | PIEKTISKAKGQPREPQVYTLPPSRDELTKNQVSLTCLVKGFYPSDIAVE |
| | | | WESNGQPENNYKTTPPVLDSDGSFFLYSKLTVDKSRWQQGNVFSCS |
| | | | VMHEALHNHYTQKSLSLSPGK |
| 112 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, C226A, | SAAK | VYYCAKLPFRFSDRGQGTLVTVSSASTEPKSSDKTHTAPPAPAPELLG |
| | C229A,P238K | | GKSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALP |
| | | | APIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |
| 113 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, C226A, | SACK | VYYCAKLPFRFSDRGQGTLVTVSSASTEPKSSDKTHTAPPCPAPELLG |
| | P238K | | GKSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALP |
| | | | APIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |
| 114 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, C229A, | SCAK | VYYCAKLPFRFSDRGQGTLVTVSSASTEPKSSDKTHTCPPAPAPELLG |
| | P238K | | GKSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALP |
| | | | APIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |
| 115 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, C226A, | SASK | VYYCAKLPFRFSDRGQGTLVTVSSASTEPKSSDKTHTAPPSPAPELLG |
| | C229S, P238K | | GKSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALP |
| | | | APIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |
| 116 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, C226S, | SSAK | VYYCAKLPFRFSDRGQGTLVTVSSASTEPKSSDKTHTSPPAPAPELLG |
| | C229A, P238K | | GKSVFLFPPKPKDTLMISRTPEVTCVVVDVSHEDPEVKFNWYVDGVE |
| | | | VHNAKTKPREEQYNSTYRVVSVLTVLHQDWLNGKEYKCKVSNKALP |
| | | | APIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |

d) dAb-Fc molecules were also generated with L234A. L235A mutations (abbreviated “LALA”) in the context of both an IgG1a and IgG1f allotype. See SEQ ID NOs: 117-118 in Table 22.

TABLE 22

| SEQ | | | |
|---|---|---|---|
| ID | | | |
| NO: | Long name | Short ID | Sequence |
| 117 | 3h56-269- | 3h56-269- | EVQLLESGGGLVQPGGSLRLSCAASGFTFRDYEMWWVRQAPGK |
| | IgG1a- | IgG1a- | GLERVSAINPQGTRTYYADSVKGRFTISRDNSKNTLYLQMNSLRAE |
| | C220S, L234A, | C220S, LALA | DTAVYYCAKLPFRFSDRGQGTLVTVSSASTEPKSSDKTHTCPPCPA |
| | L235A | | PEAAGGPSVFLFPPKPKDTLMISRTPEVTCVVVDVSHEDPEVKFN |
| | | | WYVDGVEVHNAKTKPREEQYNSTYRVVSVLTVLHQDWLNGKEY |
| | | | KCKVSNKALPAPIEKTISKAKGQPREPQVYTLPPSRDELTKNQVSLT |
| | | | CLVKGFYPSDIAVEWESNGOPENNYKTTPPVLDSDGSFFLYSKLTV |
| | | | DKSRWQQGNVFSCSVMHEALHNHYTQKSLSLSPGK |
| 118 | 3h56-269- | 3h56-269- | EVQLLESGGGLVQPGGSLRLSCAASGFTFRDYEMWWVRQAPGK |
| | IgG1f- | IgG1f- | GLERVSAINPQGTRTYYADSVKGRFTISRDNSKNTLYLQMNSLRAE |
| | C220S, L234A, | C220S- | DTAVYYCAKLPFRFSDRGQGTLVTVSSASTEPKSSDKTHTCPPCPA |
| | L235A | LALA | PEAAGGPSVFLFPPKPKDTLMISRTPEVTCVVVDVSHEDPEVKFN |
| | | | WYVDGVEVHNAKTKPREEQYNSTYRVVSVLTVLHQDWLNGKEY |
| | | | KCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLT |
| | | | VDKSRWQQGNVFSCSVMHEALHNHYTQKSLSLSPGK |

e) dAb-Fc molecules were also generated containing a single N297A mutation in the context of both an IgG1a and IgG1f allotype. See SEQ ID NOs: 119-120 in Table 23.

TABLE 23

| SEQ | | | |
|---|---|---|---|
| ID | | | |
| NO: | Long name | Short ID | Sequence |
| 119 | 3h56-269- | 3h56-269- | EVQLLESGGGLVQPGGSLRLSCAASGFTFRDYEMWWVRQAPGKGL |
| | IgG1a- | IgG1a- | ERVSAINPQGTRTYYADSVKGRFTISRDNSKNTLYLQMNSLRAEDTA |
| | C220S, N297A | C220S, | VYYCAKLPFRFSDRGQGTLVTVSSASTEPKSSDKTHTCPPCPAPELLG |
| | | N297A | GPSVFLFPPKPKDTLMISRTPEVTCVVVDVSHEDPEVKFNWYVDGVE |
| | | | VHNAKTKPREEQYASTYRVVSVLTVLHQDWLNGKEYKCKVSNKALP |
| | | | APIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |
| 120 | 3h56-269- | 3h56-269- | EVQLLESGGGLVQPGGSLRLSCAASGFTFRDYEMWWVRQAPGKGL |
| | IgG1f- | IgG1f- | ERVSAINPQGTRTYYADSVKGRFTISRDNSKNTLYLQMNSLRAEDTA |
| | C220S, N297A | C220S- | VYYCAKLPFRFSDRGQGTLVTVSSASTEPKSSDKTHTCPPCPAPELLG |
| | | N297A | GPSVFLFPPKPKDTLMISRTPEVTCVVVDVSHEDPEVKFNWYVDGVE |
| | | | VHNAKTKPREEQYASTYRVVSVLTVLHQDWLNGKEYKCKVSNKALP |
| | | | APIEKTISKAKGQPREPQVYTLPPSREEMTKNQVSLTCLVKGFYPSDIA |
| | | | VEWESNGQPENNYKTTPPVLDSDGSFFLYSKLTVDKSRWQQGNVFS |
| | | | CSVMHEALHNHYTQKSLSLSPGK |

In addition to dAb-Fc variants, a smaller set of related Fc mutants were designed to determine if similar mutations in the context of a full IgG would have similar impact on properties as in the a dAb-Fc format. All IgG variants were produced with the variable domains of the control 1F4 antibody. The sequences of the heavy chains of these variants are shown in Table 24. The sequence (SEQ ID NO: 80) of the portion of the 1F4 heavy chain including the variable region and CH1 region is italicized. For each of these variant 1F4 monoclonal antibodies, the light chain sequence was SEQ ID NO: 81 (see Table 7). The variants included:
a) single and double C226S and C229S variants. See SEQ ID NOs: 121-123 in Table 24.
b) single and double C226A and C229A variants. See SEQ ID NOs: 124-126 in Table 24.
c) P238S and P238K variants. See SEQ ID NOs: 127-128 in Table 24.
d) The C226S,C229S,P238S triple mutant was tested in the context of an IgG1f allotype. See SEQ ID NO: 129 in Table 24.
e) The N297A mutation was tested context of an IgG1f allotype. See SEQ ID NO: 130 in Table 24.

TABLE 24

| SEQ | | |
|---|---|---|
| ID | | |
| NO: | Sample ID | HC |
| 121 | 1F4-IgG1a-C226S heavy | EVQLLESGGGLVQPGGSLRLSCAASGFTFSIYAMSWVRQAPGKG |
| | chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTSPPCPAPELLGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 122 | 1F4-IgG1a-C229S heavy | EVQLLESGGGLVQPGGSLRLSCAASGFTFSIYAMSWVRQAPGKG |
| | chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPSPAPELLGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 123 | 1F4-IgG1a-C226S-C229S | EVQLLESGGGLVQPGGSLRLSCAASGFTFSIYAMSWVRQAPGKG |
| | heavy chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTSPPSPAPELLGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 124 | 1F4-IgG1a-C226A heavy | EVQLLESGGGLVQPGGSLRLSCAASGFTFSIYAMSWVRQAPGKG |
| | chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTAPPCPAPELLGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 125 | 1F4-IgG1a-C229A heavy | EVQLLESGGGLVQPGGSLRLSCAASGFTFSIYAMSWVRQAPGKG |
| | chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPAPAPELLGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 126 | 1F4-IgG1a-C226A-C229A | EVQLLESGGGLVQPGGSLRLSCAASGFTFSIYAMSWVRQAPGKG |
| | heavy chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTAPPAPAPELLGGPSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 127 | 1F4-IgG1a-P238S heavy | EVQLLESGGGLVQPGGSLRLSCAASGFTFSIYAMSWVRQAPGKG |
| | chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPCPAPELLGGSSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 128 | 1F4-IgG1a-P238K heavy | EVQLLESGGGLVQPGGSLRLSCAASGFTFSIYAMSWVRQAPGKG |
| | chain | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPCPAPELLGGKSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSRDELTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 129 | 1F4-CTf heavy chain | EVQLLESGGGLVQPGGSLRLSCAASGFTFSIYAMSWVRQAPGKG |
| | | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTSPPSPAPELLGGSSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPSREEMTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 130 | 1F4-N297A heavy chain | EVQLLESGGGLVQPGGSLRLSCAASGFTFSIYAMSWVRQAPGKG |
| | | LEWVSAISDSGGRTYFADSVRGRFTISRDNSKNTLSLQMNSLRAE |
| | | DTAVYYCAKVDYSNYLFFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPCPAPELLGGPSVFLFPPKPKDTLMISRTPEVTCVVV |
| | | DVSHEDPEVKFNWYVDGVEVHNAKTKPREEQYASTYRVVS |
| | | VLTVLHQDWLNGKEYKCKVSNKALPAPIEKTISKAKGQPRE |
| | | PQVYTLPPSREEMTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPG |

The pair of heavy chain and light chains sequences for each 1F4 mAb variant is shown in Table 25.

TABLE 25

| mAb name | HC | LC |
|---|---|---|
| 1F4-IgG1a-C226S | SEQ #: 121 | SEQ #: 80 |
| 1F4- IgG1a-S229S | SEQ #: 122 | SEQ #: 80 |
| 1F4- IgG1a-C226S-C229S | SEQ #: 123 | SEQ #: 80 |
| 1F4- IgG1a-C226A | SEQ #: 124 | SEQ #: 80 |
| 1F4- IgG1a-C229A | SEQ #: 125 | SEQ #: 80 |
| 1F4- IgG1a-C226A-C229A | SEQ #: 126 | SEQ #: 80 |
| 1F4- IgG1a-P238S | SEQ #: 127 | SEQ #: 80 |
| 1F4- IgG1a-P238K | SEQ #: 128 | SEQ #: 80 |
| 1F4-CTf | SEQ #: 129 | SEQ #: 80 |
| 1F4-N297A | SEQ #: 130 | SEQ #: 80 |

All the 1F4-IgG variants were produced with the wild type Cys220 residue intact to pair with the C-terminal Cys residue of the antibody light chain.

### Example 5: Characterization of 1F4 Control Antibodies with Variant Fc Domains

To characterize the FcgR binding properties of Fc-engineered 1F4 antibody molecules, SPR experiments were performed by testing 1 μM or 10 UM purified antibody analytes binding to anti-His captured FcgRs surfaces, as described in Example 2. The binding responses were analyzed and represented by % Rmax values; the results are shown in Table 26.

TABLE 26
% Rmax data for 1 μM or 10 μM 1F4-IgG1f antibodies
binding to anti-His Fab captured hFcgR-His proteins.

| | | Anti-His Fab captured FcgR | | | | | |
|---|---|---|---|---|---|---|---|
| | | High | | | | | |
| | | affinity | Low Affinity FcgR | | | | |
| | Conc | FcgR | hCD32a- | hCD32a- | | hCD16a- | hCD16b- |
| Sample | (μM) | hCD64 | H131 | R131 | hCD32b | V158 | NA2 |
| 1F4-IgG1f | 1 | 63% | 29% | 20% | 5% | 33% | 14% |
| 1F4-IgG1.3f | 1 | 1% | 0% | 1% | 0% | 0% | 0% |
| 1F4-N297A | 1 | 39% | 0% | 0% | 0% | 0% | 0% |
| 1F4-CTf | 1 | 61% | 0% | 0% | 0% | 1% | 0% |
| 1F4-CT | 1 | 61% | 0% | 0% | 0% | 1% | 0% |
| 1F4-IgG1a-C226S | 1 | 64% | 23% | 16% | 4% | 26% | 8% |
| 1F4-IgG1a-S229S | 1 | 64% | 19% | 16% | 4% | 20% | 6% |
| 1F4-IgG1a-C226S- | 1 | 63% | 2% | 2% | 0% | 4% | 1% |
| C229S | | | | | | | |
| 1F4-IgG1a-P238S | 1 | 63% | 2% | 4% | 1% | 2% | 0% |
| 1F4-IgG1a-C226A | 1 | 64% | 24% | 17% | 4% | 26% | 9% |
| 1F4-IgG1a-C229A | 1 | 64% | 21% | 17% | 4% | 21% | 6% |
| 1F4-IgG1a-C226A- | 1 | 63% | 3% | 3% | 1% | 6% | 1% |
| C229A | | | | | | | |
| 1F4-IgG1a-P238K | 1 | 42% | 0% | 0% | 0% | 0% | 0% |
| 1F4-IgG1f | 10 | 65% | 60% | 51% | 24% | 54% | 38% |
| 1F4-IgG1.3f | 10 | 10% | 1% | 8% | 3% | 1% | 1% |
| 1F4-N297A | 10 | 56% | 0% | 0% | 0% | 1% | 0% |
| 1F4-CTf | 10 | 65% | 1% | 2% | 1% | 2% | 0% |
| 1F4-CT | 10 | 65% | 1% | 2% | 1% | 2% | 0% |
| 1F4-IgG1a-C226S | 10 | 66% | 55% | 48% | 20% | 51% | 30% |
| 1F4-IgG1a-S229S | 10 | 66% | 51% | 48% | 23% | 45% | 23% |
| 1F4-IgG1a-C226S- | 10 | 66% | 12% | 12% | 4% | 18% | 4% |
| C229S | | | | | | | |
| 1F4-IgG1a-P238S | 10 | 65% | 15% | 21% | 7% | 9% | 2% |
| 1F4-IgG1a-C226A | 10 | 66% | 56% | 48% | 20% | 51% | 30% |
| 1F4-IgG1a-C229A | 10 | 66% | 53% | 48% | 22% | 46% | 25% |
| 1F4-IgG1a-C226A- | 10 | 65% | 20% | 17% | 5% | 23% | 6% |
| C229A | | | | | | | |
| 1F4-IgG1a-P238K | 10 | 58% | 0% | 0% | 0% | 0% | 0% |

These data show that, compared to the wild-type IgG1f antibody (1F4-IgG1f), the single Cys→Ser mutation at position 226 (1F4-IgG1a-C226S) or position 229 (1F4-IgG1a-C229S) in the hinge region have minimal impact on FcgR binding. A double C226S,C229S mutant (1F4-IgG1a-C226S-C229S) has significantly weaker binding responses towards all of the low affinity FcgR proteins; however, the binding responses are still significantly stronger than that of the 1F4-CT molecules. These data suggest that the additional P238S mutation in the 1F4-CT molecules further contributes to the reduced FcgR binding.

Single C226A (1F4-IgG1a-C226A) or C229A (1F4-IgG1a-C229A) mutants bound FcgRs similarly to the single C226S or C229S mutants; and likewise the C226A,C229A double mutant (1F4-IgG1a-C226A-C229A) bound FcgRs similarly to the C226S,C229S double mutant (1F4-IgG1a-C226S-C229S). Ala mutations at these sites would prevent inter-heavy chain disulfide bond formation, similar to Ser mutations at these sites. Unlike Ser mutations, however, the Ala mutations would not be O-glycosylation sites. Therefore, these data suggest that O-glycosylation at S226 and/or S229 does not have a significant impact on FcgR binding.

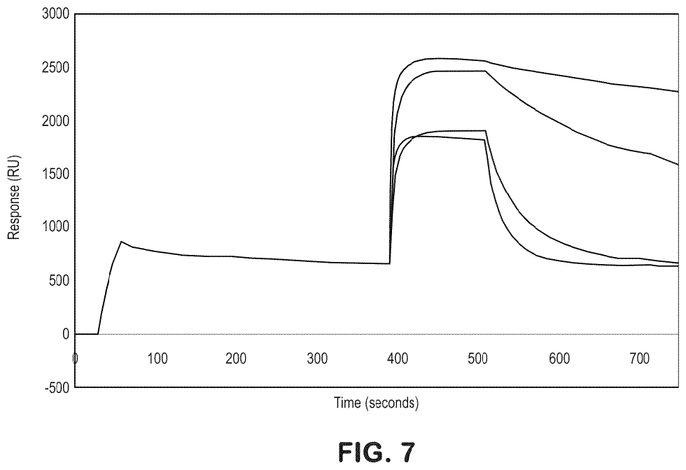

The P238K and N297A variants (1F4-IgG1a-P238K and 1F4-N297A, respectively) demonstrate the weakest binding responses towards the low affinity FcgRs, demonstrating essentially no detectable binding signal towards hCD32a-H131, hCD32a-R131, hCD32b, hCD16a-V158 or hCD16b-NA2. The 1F4-IgG1a-P238K variant also demonstrated weaker FcgR binding than the 1F4-IgG1a-P238S variant, suggesting that Lys at position 238 is more effective at disrupting FcgR binding than Ser at that position. In addition, the SPR sensorgram data showed that the dissociation rates for 1F4-IgG1f-N297A and 1F4-IgG1a-P238K binding to hCD64 were significantly faster than for 1F4-IgG1f or 1F4-CT. See FIG. 7.

The thermal stability of the Fc-variant 1F4 antibodies was characterized by DSC, as described in Example 3. Thermal transitions were assigned to either CH2 domain, the CH3 domain, or the Fab domain based on the well-characterized thermal denaturation profiles for IgG molecules, and the best fit Tm values are summarized in Table 27.

TABLE 27
Thermal melting temperature (Tm) values
for 1F4 antibodies as determined by DSC.

| | Tm1 (° C.) | Tm2 (° C.) | Tm3 (° C.) |
|---|---|---|---|
| Sample | (CH2) | (Fab) | (CH3) |
| 1F4-IgG1f | 72.2 | 74.7 | 82.8 |
| 1F4-IgG1.3f | 68.5 | 73.8 | 82.6 |
| 1F4-N297A | 60.5 | 72.6 | 82.4 |
| 1F4-CTf | 54.3 | 71.9 | 82.1 |
| 1F4-CT | 55.1 | 72.1 | 82.8 |
| 1F4-IgG1a-C226S | 70.3 | 72.9 | 82.9 |
| 1F4-IgG1a-C229S | 69.9 | 72.8 | 82.7 |
| 1F4-IgG1a-C226S- | 64.8 | 71.6 | 82.7 |
| C229S | | | |
| 1F4-IgG1a-P238S | 62.4 | 71.9 | 83.0 |
| 1F4-IgG1a-C226A | 70.3 | 72.9 | 82.9 |
| 1F4-IgG1a-C229A | 70.0 | 72.7 | 82.5 |
| 1F4-IgG1a-C226A- | 66.0 | 71.6 | 82.7 |
| C229A | | | |
| 1F4-IgG1a-P238K | 64.0 | 71.9 | 83.1 |

The Fab domain of the 1F4 antibodies have a fit Tm between 71.6° C. and 74.7° C. The CH3 domains of all molecules melted between 82.1° C. and 83.1° C., which is typical for a wild type (unmodified) IgG1 CH3 domain. The CH2 domains were the least stable domains of the antibody, and the melting temperatures were different for different mutants, suggesting that the mutations in the hinge/CH2 region impact the thermal stability of the CH2. The Tm values for the CH2 domain of 1F4-CTf (54.3° C.), and 1F4-CT (55.1° C.) were less than 1° C. different, suggesting that the IgG1 allotype has minimal impact on the thermal stability of the CH2 domain. However, these CH2 domains were dramatically destabilized by ˜17-18° C. relative to the wild-type CH2 domain of 1F4-IgG1f (72.2° C.). These data are consistent with the low thermal stability observed for the CH2/dAb domains of 3h56-269-CT.

Fc mutants with single Cys→Ser mutations in the hinge region had modestly lower CH2 domain stability compared to wild-type IgG1f, with CH2 domain Tm values for 1F4-IgG1a-C226S of 70.3° C., and for 1F4-IgG1a-C229S of 69.9° C. Mutation of both the hinge Cys residues to Ser further reduced the CH2 domain Tm to 64.8° C. for 1F4-IgG1a-C226S,C229S. The single P238S mutation also reduced the CH2 domain stability (62.4° C.) compared to wild-type 1F4-IgG1f. Therefore, these data show that none of the three individual mutations in 1F4-CT is solely responsible for the low CH2 domain stability, but rather that the combination of all three mutations (C226S, C229S, P238S) leads to the dramatic destabilization of the CH2 domain.

The single Cys→Ala mutants in the hinge 1F4-IgG1a-C226A and 1F4-IgG1a-C229A have nearly identical CH2 domain Tm values as the Cys→Ser mutants at these positions, and the double mutant 1F4-C226A,C229A has a CH2 domain Tm that is modestly (1.2° C.) more stable than that of the double Cys→Ser mutant 1F4-C226S,C229S. The CH2 domain of 1F4-IgG1a-P238K (Tm=64.0° C.) is 1.6° C. more stable than the Ser mutant at this position, 1F4-IgG1a-P238S (Tm=62.4° C.).

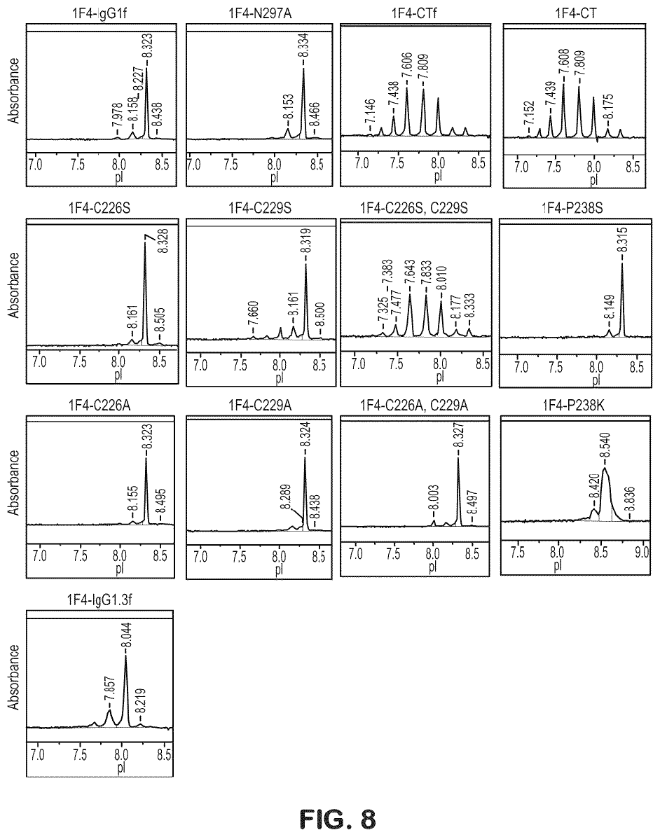

To determine the impact of the hinge/Fc mutations on sample heterogeneity, the 1F4-IgG molecules were characterized by icIEF, as described in Example 3. The icIEF profile for the 1F4-IgG1f protein was typical for a monoclonal IgG1 antibody, with a main peak of 79.7% abundance, and ˜2-4 acidic or basic variants in much lower abundance. See FIG. 8. Like the domain antibody with the CT Fc domain (3h56-269-CT), the icIEF profile for the 1F4-CT molecules were heterogeneous, consisting of at least 8 distinct charge variants and no obvious dominant species. This heterogeneity is likely related to the glycan heterogeneity observed by mass spectrometry (Table 17), as discussed above.

The icIEF data for the double Cys→Ser variant 1F4-IgG1a-C226S,C229S was similar to that of the 1F4-CT molecule, showing the presence of numerous different charge variants with no distinct main peak, whereas the data for the single mutants C226S, C229S and P238S were all of similar complexity to 1F4-IgG1f. These data suggest that the high levels of O-linked sialylated glycosylation in the hinge/Fc region requires both the C226S and C229S mutations, which disrupt both the inter heavy chain hinge disulfide bonds.

The icIEF data for 1F4-IgG1.3f, 1F4-N297A, 1F4-IgG1a-P238K and each of the single and double Ala mutants, demonstrated homogeneity similar to that of 1F4-IgG1f, each consisting of a main peak of 62-80% abundance with ˜2-3 acidic or basic variants in smaller abundance.

Collectively, the icIEF data show that all molecules that have both hinge Cys226 and Cys229 residues mutated to Ser have significantly higher heterogeneity than the other variants.

### Summary of the Control Antibody Data:

The SPR, DSC, icIEF, and mass spec data for the 1F4-IgG molecules provides insight into the role of the C226, C229 and P238 mutations on the FcgR binding, thermal stability, and heterogeneity of the CT Fc domain.

The single hinge C226S and C229S mutants only modestly reduced thermal stability, had similar heterogeneity, and similar FcgR binding as 1F4-IgG1f, whereas the double C226S,C229S hinge mutant had significantly lower thermal stability, increased heterogeneity, and reduced FcgR binding compared to 1F4-IgG1f. The single P238S mutation had similar impact on reducing thermal stability and FcgR binding as the double C226S,C229S mutant, but did not increase heterogeneity. Combination of the C226S,C229S hinge mutations together with P238S to yield the full 1F4-CT molecule, had similar heterogeneity as C226S,C229S alone, but further reduced thermal stability and FcgR binding. Collectively, these data suggest that the combined C226S,C229S mutations plus P238S each contribute to the reduced thermal stability and reduced FcgR binding compared to wild-type Fc, and that the primary sites for O-linked glycosylation are on the mutated hinge S226 and Ser229 residues.

The single and double Cys→Ala mutations at positions 226 and 229 in the hinge region have similar thermal stability and FR binding as Cys→Ser mutants at those sites. However, the C226A,C229A mutant lacks the O-linked glycosylation sites on Ser residues and does not have the high heterogeneity observed with the C226S,C229S mutant. This suggests that the O-linked glycosylation in the hinge region does not have a significant impact on FcgR binding.

The 1F4-IgG1a-P238K mutant demonstrated weaker FcgR binding than 1F4-IgG1a-P238S, while having similar heterogeneity and superior thermal stability compared to 1F4-IgG1a-P238S. Compared to the 1F4-CT molecules, 1F4-IgG1a-P238K demonstrated weaker FcgR binding, improved thermal stability, and superior homogeneity. Therefore, the single P238K mutation unexpectedly provided all three of the properties that were desired when designing this set of hinge/Fc variants (comparable or weaker FcgR binding, superior thermal stability and reduced heterogeneity compared to 1F4-CT).

The 1F4-N297A molecule demonstrated lower CH2 domain thermal stability and weaker FcgR binding compared to 1F4-IgG1f, which are properties consistent with literature reports for other IgG1 antibodies containing N297A mutation. The homogeneity for 1F4-N297A was similar to that of 1F4-IgG1f.

Overall, the 1F4-IgG molecules demonstrating the weakest FcgR binding were 1F4-IgG1a-P238K, 1F4-N297A and the 1F4-CT molecules. Of these, 1F4-IgG1a-P238K and 1F4-N297A had superior thermal stability and homogeneity compared to 1F4-CT, with 1F4-IgG1a-P238K having superior thermal stability over 1F4-N297A. Consequently, the P238K and N297A isotypes were selected as leads for further characterization.

### Example 6: Characterization of dAb-Fc Antibodies with Variant Fc Domains

The FcgR binding SPR, DSC, icIEF, and MS data for the 1F4-IgG molecules provided considerable insight into the regions and mutations in the CT isotype which contribute to FcgR binding, stability and heterogeneity, as discussed in Example 5. Therefore, these data were used to prioritize a subset of dAb-Fc isotype variants for expression as small scale expression supernatants, for screening by SPR for FcgR binding.

For example, the P238K and N297A single mutants in the 1F4 antibodies provided favorably weak FcgR binding properties, while maintaining superior thermal stability and homogeneity over the CT isotype molecules. Therefore, 3h56-269-IgG1a-C220S,P238K and 3h56-269-IgG1f-C220S,N297A molecules were included in the dAb-Fc analysis.

In addition, the superior homogeneity but similar thermal stability and FcgR binding properties of the C226A,C229A double mutant compared to the C226S,C229S double mutant, raises the possibility that a C226A,C229A double mutant combined with P238S or P238K may possess the desired weak FcgR binding, but without the high heterogeneity and O-linked glycan that is a consequence of mutating C226,C229 each to Ser. As a result, variants were selected (i.e., 3h56-269-IgG1a-C220S,C226A,C229A,P238S and 3h56-269-IgG1a-C220S,C226A,C229A,P238K variants) to further investigate.

Since the C220 residue in the 1F4-IgG1 molecules was retained as the wild-type Cys to ensure native disulfide bonding with the antibody light chain, the impact of mutations at position 220 were not investigated in the context of the 1F4-IgG molecules. However, since the dAb-Fc antibody polypeptides do not have a light chain, the C220 residue would either form a free Cys, or potentially disulfide bond with another free Cys such as the C220 residue of the partner dAb-Fc chain. Therefore, a subset of C220 mutants in the dAb-Fc variant analysis were included to determine the impact of mutations at that position on the FcgR binding properties of the molecule.

For comparison, the double L234A,L235A (LALA) mutant was generated in the context of both IgG1a and IgG1f allotypes.

In addition to methods described previously, methods used in this example include the following.

Inhibition of CD40L induced human B cell proliferation: Human tonsillar B cells were obtained from pediatric patients during routine tonsillectomy and isolated by mincing and gently mashing the tissue, passing the cells through a screen and isolating mononuclear cells with density gradient separation using human Lympholyte®-H separation media (Cedarlane Labs, Burlington, ON). Mononuclear cells were collected from the interface, washed, and rosetted with sheep red blood cells (SRBC, Colorado Serum Company; Denver, CO) for one hour at 4° C., followed by density gradient separation to remove T cells. Cells were again washed and resuspended in RPMI containing 10% FBS (complete media). Titrations of antibodies were made in complete media, and added in triplicate to 96-well round bottom (RB) plates. 1×10⁵ tonsillar human B cells were added and stimulated with either soluble IZ-hCD40L (2 μg/mL), or with Chinese hamster ovary cells stably transfected with human CD40L (CHO-hCD40L) irradiated with 10,000 rads, and plated at 2×10³ cells/well, in a final volume of 200 μL in each well. Plates were incubated at 37° C. and 5% CO2 for 72 hours, labeled for the last 6 hours with 0.5 μCi of ³[H]-thymidine per well, harvested, and counted by liquid scintillation. B cell proliferation was quantitated based on thymidine incorporation.

Results—SPR: The selected dAb-Fc variants were expressed as small scale supernatants, captured on immobilized protein A Biacore™ SPR sensor chip surface, and tested for binding to purified FcgR analytes (μM), as described in Example 2. The data are shown in Table 28.

TABLE 28
% Rmax data for 1 μM FcgRs binding to protein A captured dAb-Fc molecules.

| | Analyte: | | | | | |
|---|---|---|---|---|---|---|
| Protein A- | | hCD32a- | hCD32a- | | hCD16a- | hCD16b- |
| captured dAb-Fc | hCD64 | H131 | R131 | hCD32b | V158 | NA2 |
| 3h56-269-CT (n = 4) | 88% | 0% | 0% | −1% | −4% | −2% |
| | 90% | 0% | 0% | 0% | −2% | −1% |
| | 92% | 0% | 1% | 0% | −1% | −2% |
| | 92% | 0% | 0% | 0% | 0% | −1% |
| 3h56-269-IgG1a- | 94% | 37% | 34% | 12% | 58% | 13% |
| CCCP | | | | | | |
| 3h56-269-IgG1a- | 101% | 40% | 37% | 14% | 66% | 16% |
| SCCP | | | | | | |
| 3h56-269-IgG1a- | 103% | 6% | 13% | 4% | 8% | −2% |
| SCCS | | | | | | |
| 3h56-269-IgG1a- | 84% | 3% | 5% | 1% | 11% | −1% |
| SSSP | | | | | | |
| 3h56-269-IgG1a- | 91% | 8% | 9% | 3% | 19% | 1% |
| SAAP | | | | | | |
| 3h56-269-IgG1a- | 97% | 1% | 2% | 0% | 0% | −3% |
| SAAS | | | | | | |
| 3h56-269-IgG1a- | 51% | 0% | 0% | −1% | −2% | −2% |
| SCCK | | | | | | |
| 3h56-269-IgG1a- | 41% | 0% | 0% | −1% | −1% | −1% |
| SSSK | | | | | | |
| 3h56-269-IgG1a- | 41% | −1% | 0% | −1% | −2% | −3% |
| SAAK | | | | | | |
| 3h56-269-IgG1a- | 44% | 0% | 0% | −1% | −1% | −2% |
| C220S, N297A | | | | | | |
| 3h56-269-IgG1f- | 47% | −1% | −1% | −1% | −2% | −3% |
| C220S-N297A | | | | | | |
| 3h56-269-IgG1a- | 28% | 1% | 1% | 0% | 14% | 0% |
| C220S, LALA | | | | | | |
| 3h56-269-IgG1f- | 29% | 1% | 1% | 0% | 14% | 0% |
| C220S-LALA | | | | | | |

The FcgR binding SPR data for the 3h56-269-IgG1a-C220S variant were similar to that for 3h56-269-IgG1a, suggesting that the C220S mutation has minimal impact on FcgR binding. This mutation may however be favored from a developability perspective in the dAb-Fc format, because it would remove a potentially reactive thiol group which could be a risk for heterogeneity during manufacturing or shelf life.

The FcgR binding SPR data for the other dAb-Fc molecules agreed well with the data for the 1F4-IgG variants. For example, all of the variants containing P238K or N297A demonstrated weaker binding to hCD64 as compared to wild type, and essentially undetectable binding to all of the other FcgRs. The P238K and N297A variants also demonstrated weaker hCD64 binding than 3h56-269-CT, similar to what was observed for the analogous 1F4-IgG variants. Also like the 1F4-IgG variants, the single P238S mutation or double C226S/C229S mutation reduced FcgR binding, but less so than the combination of these three mutations (3h56-269-CT). Also, the mutant with both hinge Cys mutated to Ala (3h56-269-IgG1a-C220S,C226A,C229A) demonstrated similar FcgR binding as the double Cys to Ser hinge variant (3h56-269-IgG1a-C220S,C226S,C229S). Addition of the P238S mutation also further reduced FcgR binding (3h56-269-IgG1a-C220S, C226A, C229A, P238S), similar to what was observed with the 1F4-IgG molecules.

The LALA variants that were tested had significantly reduced FcgR binding compared to wild type, in particular, and demonstrated the weakest hCD64 binding of any of the variants tested. However, they demonstrated stronger hCD16a-V158 binding than 3h56-269-CT, or any of the P238K or N297A molecules.

Based on SPR data obtained with the dAb-Fc supernatants, the dAb-Fc variants with the weakest binding to the low affinity FcgRs were selected for purification and further characterization. These variants include: 3h56-269-IgG1a-C220S,C226A,C229A,P238S, 3h56-269-IgG1a-C220S,C226A,C229A,P238K, 3h56-269-IgG1a-C220S,P238K, 3h56-269-IgG1f-C220S,N297A. All four molecules were shown to bind with high affinity to the CD40 target using SPR. The data are shown in Table 29.

TABLE 29
SPR data for binding of dAb-Fc molecules to human CD40.

| | | | | dAb-Fc binding to |
|---|---|---|---|---|
| | hCD40monomer binding to | | | immobilized |
| | protein A captured dAb-Fc | | | hCD40-Fc surface |
| | surface (Affinity) | | | (Avidity) |
| Sample | ka (1/Ms) | kd (1/s) | KD (nM) | KDₐₚₚₐᵣₑₙₜ (nM) |
| 3h56-269-IgG1a- | 2.8E+04 | 1.6E−04 | 5.9 | <1 |
| C220S, C226A, C229A, P238S | | | | |
| 3h56-269-IgG1a- | 3.0E+04 | 1.4E−04 | 4.6 | <1 |
| C220S, C226A, C229A, P238K | | | | |
| 3h56-269-IgG1a- | 2.5E+04 | 1.3E−04 | 4.9 | <1 |
| C220S, P238K | | | | |
| 3h56-269-IgG1f- | 2.7E+04 | 1.4E−04 | 5.0 | <1 |
| C220S, N297A | | | | |

Binding of the purified dAb-Fcs binding to FcgRs was assessed by SPR, as described in Example 2. The data for the dAb-FCs as well as the 1F4 antibody controls are shown in Table 30.

TABLE 30
% Rmax data for 1 μM or 10 μM dAb-Fc molecules or 1F4-IgG1f
antibodies binding to anti-His Fab captured hFcgR-His proteins.

| | | Anti-His Fab captured FcgR | | | | | |
|---|---|---|---|---|---|---|---|
| | | High | | | | | |
| | | affinity | Low Affinity FcgR | | | | |
| | Conc | FcgR | hCD32a- | hCD32a- | | hCD16a- | hCD16B- |
| Sample | (μM) | hCD64 | H131 | R131 | hCD32b | V158 | NA2 |
| 1F4-IgG1f | 1 | 63% | 30% | 21% | 4% | 35% | 13% |
| 1F4-IgG4.1 | 1 | 63% | 11% | 14% | 9% | 3% | −1% |
| 1F4-IgG1.3f | 1 | 3% | 0% | 1% | −1% | −1% | −1% |
| 1F4-CT | 1 | 62% | 0% | −1% | −1% | 0% | −1% |
| 1F4-IgG1a- | 1 | 63% | 3% | 2% | −1% | 5% | 0% |
| C226A-C229A | | | | | | | |
| 1F4-N297A | 1 | 45% | 0% | −1% | −1% | 0% | −1% |
| 1F4-IgG1a-P238K | 1 | 47% | 0% | −1% | −1% | 0% | −1% |
| 3h56-269-IgG4.1 | 1 | 67% | 24% | 28% | 18% | 5% | −1% |
| 3h56-269-IgG1.3f | 1 | 10% | 0% | 7% | 1% | −1% | −2% |
| 3h56-269-CT | 1 | 70% | 0% | 0% | −2% | 0% | −2% |
| 3h56-269-IgG1a- | 1 | 68% | 1% | 1% | −1% | 0% | −2% |
| SAAS | | | | | | | |
| 3h56-269-IgG1a- | 1 | 60% | −1% | −1% | −2% | −1% | −3% |
| SAAK | | | | | | | |
| 3h56-269-IgG1f- | 1 | 58% | −1% | −1% | −2% | −1% | −3% |
| C220S, N297A | | | | | | | |
| 3h56-269-IgG1a- | 1 | 63% | −1% | −1% | −2% | −1% | −3% |
| C220S,P238K | | | | | | | |
| 1F4-IgG1f | 10 | 65% | 61% | 53% | 25% | 55% | 36% |
| 1F4-IgG4.1 | 10 | 64% | 37% | 43% | 33% | 16% | 4% |
| 1F4-IgG1.3f | 10 | 13% | 0% | 9% | 3% | 0% | −1% |
| 1F4-CT | 10 | 65% | 0% | 1% | 0% | 0% | −1% |
| 1F4-IgG1a- | 10 | 65% | 21% | 18% | 4% | 24% | 5% |
| C226A-C229A | | | | | | | |
| 1F4-N297A | 10 | 57% | 0% | 0% | −1% | 0% | −1% |
| 1F4-IgG1a-P238K | 10 | 60% | −1% | −1% | −1% | −1% | −2% |
| 3h56-269-IgG4.1 | 10 | 68% | 61% | 65% | 54% | 26% | 5% |
| 3h56-269-IgG1.3f | 10 | 35% | 5% | 36% | 17% | 4% | 1% |
| 3h56-269-CT | 10 | 70% | 4% | 9% | 3% | 5% | −2% |
| 3h56-269-IgG1a- | 10 | 67% | 9% | 16% | 4% | 7% | −3% |
| SAAS | | | | | | | |
| 3h56-269-IgG1a- | 10 | 64% | −3% | −1% | 4% | −1% | −5% |
| SAAK | | | | | | | |
| 3h56-269-IgG1f- | 10 | 60% | −5% | 1% | −7% | 2% | −7% |
| C220S, N297A | | | | | | | |
| 3h56-269-IgG1a- | 10 | 63% | −5% | 0% | −6% | −1% | −7% |
| C220S, P238K | | | | | | | |

SPR data for the purified dAb-Fcs (with 1F4 antibody controls) are consistent with the dAb-Fc supernatant data, showing that the CT, N297A and P238K variants have the weakest binding to the low affinity FcgRs. See Table 30. This trend is consistent in both the 1F4 antibody and dAb-Fc formats. In fact, in the dAb-Fc format, at the highest concentration tested, 3h56-269-IgG1a-C220S,P238K, 3h56-269-IgG1a-C220S,C226A,C229A,P238K, and 3h56-269-IgG1f-C220S,N297A all demonstrated even weaker FcgR binding responses than the 3h56-269-CT.

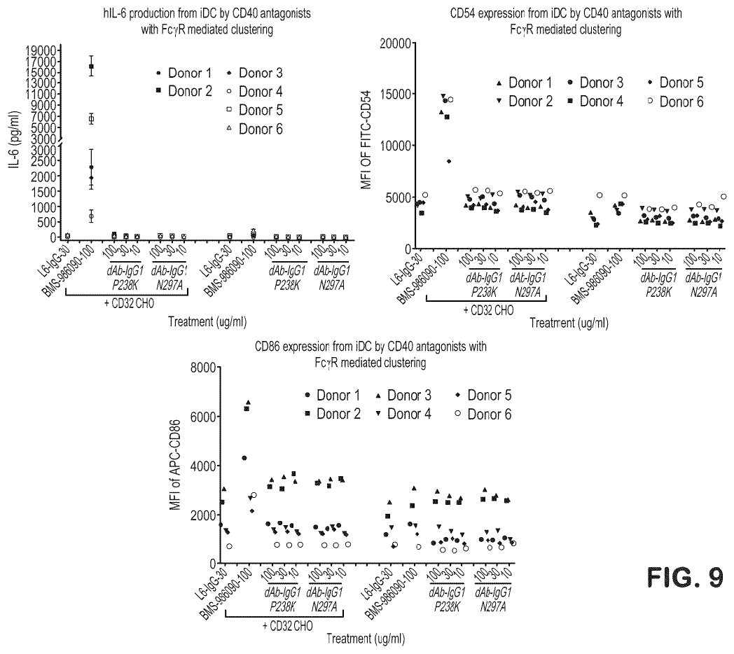

Results—iDC Activation: The 3h56-269-IgG1a-C220S,P238K and 3h56-269-IgG1f-N297A molecules were tested for the ability to activate iDC alone or with CD32-mediated clustering/cross-linking, as described in Example 1. The data shows that these mutations in the IgG1 Fc tail could eliminate any iDC activation, rendering the anti-CD40 dAb-Fc molecules inert in these assays of iDC activation. See FIG. 9. No activation of iDC as measured by cytokine production and CD86 and CD54 upregulation, was observed for either fusion protein either alone or with CD32 mediated clustering, highlighting the potential of these mutations to produce CD40 antagonists without potential for immune activation. In these same donors, modest increases in at least one measure of iDC activation was observed in iDCs from 2 of the 6 samples tested when stimulated with 3h-59-269-IgG4.1 alone, and all 6 of the donors when CD32 mediated clustering/cross-linking was included.

Results—Inhibition of CD40L induced human B cell proliferation: Despite the differential activity of the fusion proteins with different Fc tails, these changes do not influence the ability to inhibit CD40L mediated activation of immune cells, such as B cells. This is exemplified by the activity of 3h-59-269-IgG1a-P238K and 3h-59-269-IgG1f-N297A fusions. B cell proliferation stimulated by both soluble CD40L trimer and by CD40L expressing CHO cells is potently and similarly inhibited by 3h-59-269-IgG1-P238K, or 3h-59-269-IgG1-N297A (Table 31).

TABLE 31
Changes in the Fc tail do not affect the potency of anti-
CD40 dAb to inhibit CD40L induced B cell proliferation.

Stimulation

| IC50 (ng/ml) | CD40L-IZ trimer | CD40L expressing CHO |
|---|---|---|
| dAb-CT | 2.8 +/− 1.2 (n = 6) | 34.3 +/− 3.8 (n = 4) |
| dAb-N297A | 2.8 +/− 0.8 (n = 4) | 39.9 +/− 4.2 (n = 2) |
| dAb-P238K | 2.3 +/− 0.6 (n = 4) | 34.6 +/− 5.5 (n = 2) |

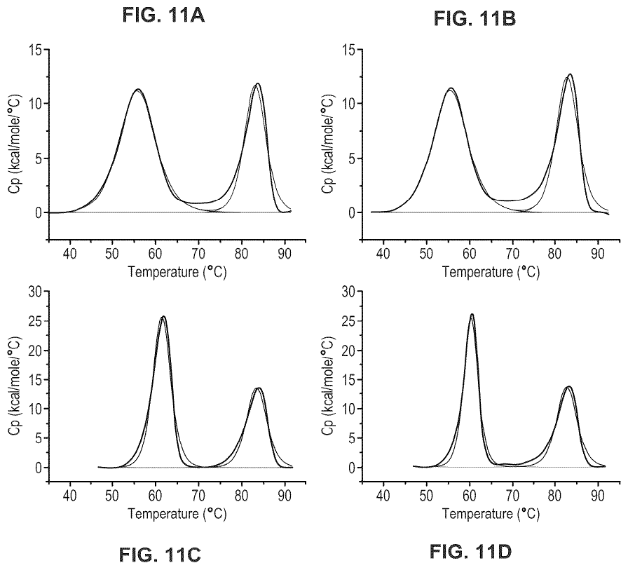

Results—DSC: The thermal stability of four purified dAb-Fcs which demonstrated low FcgR binding were characterized by DSC, as described in Example 3. Like the previously characterized IgG1-type dAb-Fc molecules, all four new molecules demonstrate a transition near 83° C., which is characteristic of the CH3 domain of a human IgG1 Fc domain, with a lower temperature transitions assigned to the dAb and CH2 domains. These data are in Table 32. See also FIGS. 11A-11D.

TABLE 32
Thermal melting temperature (Tm) values for
dAb-Fc molecules as determined by DSC.

| | Tm1 − dAb and | Tm2 − CH3 |
|---|---|---|
| Sample | CH2 domains (° C.) | domain (° C.) |
| 3h56-269-IgG1f-C220S, N297A | 60.5 | 83.0 |
| 3h56-269-IgG1a-C220S, P238K | 61.5 | 83.4 |
| 3h56-269-IgG1a- | 55.8 | 83.1 |
| C220S, C226A, C229A, P238S | | |
| 3h56-269-IgG1a- | 55.7 | 83.0 |
| C220S, C226A, C229A, P238K | | |

The lower temperature transition for both the 3h56-269-IgG1a-C220S,C226A,C229A,P238S and 3h56-269-IgG1a-C220S,C226A,C229A,P238K variants had a low Tₒₙₛₑₜ near ˜40° C., and broad unfolding transition with Tm1 value of 55.7-55.8° C., similar to the previous data observed for 3h56-269-CT. The thermal stability of 3h56-269-IgG1a-C220S,P238K and 3h56-269-IgG1f-C220S,N297A was considerably more favorable, with Tₒₙₛₑₜ near ˜50° C., and Tm1 values of 60.5° C. (3h56-269-IgG1f-C220S,N297A) and 61.5° C. (3h56-269-IgG1a-C220S,P238K).

Results—Accelerated Stability Studies: The physical stability of the dAb-Fc molecules was studied under accelerated stress conditions. First, a study was conducted to compare the physical stability of the four new optimized variants (3h56-269-IgG1a-C220S,C226A,C229A,P238S, 3h56-269-IgG1a-C220S,C226A,C229A,P238K, 3h56-269-IgG1a-C220S,P238K and 3h56-269-IgG1f-C220S,N297A) directly to the original 3h56-269-CT molecule. Here samples were prepared at 15 mg/ml in 20 mM acetate, 250 mM sucrose at pH 5.0, and incubated at 40° C. for four weeks. Aliquots were removed at the onset of the study (time zero, t0), 1 week, and 4 weeks, and subjected to analytical SEC analysis (aSEC). The data are shown in Table 33.

TABLE 33
Percentage of high molecular weight (HMW) species as
determined by analytical size exclusion chromatography
for dAb-Fc samples incubated at 40° C. for 4 weeks.

| | Conc | | | |
|---|---|---|---|---|
| Sample | (mg/ml) | t0 | 1 week | 4 weeks |
| 3h56-269-CT | 15 | 0.5% | 2.5% | 16.6% |
| 3h56-269-IgG1a- | 15 | 0.0% | 2.2% | 14.9% |
| C220S, C226A, C229A, P238S | | | | |
| 3h56-269-IgG1a- | 15 | 0.0% | 0.8% | 13.4% |
| C220S, C226A, C229A, P238K | | | | |
| 3h56-269-IgG1f-C220S, N297A | 15 | 0.0% | 0.4% | 1.2% |
| 3h56-269-IgG1a-C220S, P238K | 15 | 0.0% | 0.3% | 1.0% |

These data showed large increases (e.g., 0.5% to greater than 13%) of the HMW species for 3h56-269-IgG1a-C220S,C226A,C229A,P238S, 3h56-269-IgG1a-C220S,C226A,C229A,P238K, and 3h56-269-CT, but only small increases (e.g., 0% to 1.2%) for 3h56-269-IgG1a-C220S,P238K and 3h56-269-IgG1f-C220S,N297A.

To further compare the physical stability of the four optimized dAb-Fc proteins, a second study was conducted at higher concentration. Samples were prepared at 70 mg/ml for 3h56-269-IgG1a-C220S,C226A,C229A,P238K, 3h56-269-IgG1a-C220S,P238K and 3h56-269-IgG1f-C220S,N297A, or 30 mg/ml for 3h56-269-IgG1a-C220S,C226A,C229A,P238S (the latter sample had a lower concentration due to material limitations arising from lower expression levels and lower yield after purification) in 20 mM acetate, 250 mM sucrose pH 5.0 and incubated at either 40° C., 25° C., or under refrigerated temperature (4° C.) for four to 12 weeks, with aliquots removed at various time point sand subjected to analytical SEC analysis. The data are shown in Table 34.

TABLE 34
Percentage of high molecular weight (HMW) species as determined
by analytical size exclusion chromatography for dAb-Fc samples
incubated at 4° C., 25° C., 32° C. or 40° C. for 4 weeks.

| | Conc | Temp. | | | |
|---|---|---|---|---|---|
| Sample | (mg/ml) | (° C.) | t0 | 11 days | 4 weeks |
| 3h56-269-IgG1a- | 70 | 4 | 0.4% | 0.2% | 0.5% |
| C220S, C226A, C229A, | | 25 | 0.4% | 0.5% | 0.7% |
| P238S | | 40 | 0.4% | 6.9% | 21.1% |
| 3h56-269-IgG1a- | 70 | 4 | 0.3% | 0.4% | 0.5% |
| C220S, C226A, C229A, | | 25 | 0.3% | 0.7% | 3.8% |
| P238K | | 40 | 0.3% | 14.6% | 30.8% |
| 3h56-269-IgG1f- | 70 | 4 | 0.1% | 0.0% | 0.4% |
| C220S, N297A | | 25 | 0.1% | 0.3% | 0.8% |
| | | 40 | 0.1% | 1.9% | 3.5% |
| 3h56-269-IgG1a- | 70 | 4 | 0.1% | 0.1% | 0.3% |
| C220S, P238K | | 25 | 0.1% | 0.2% | 0.6% |
| | | 40 | 0.1% | 1.7% | 3.2% |

These data also showed larger to much larger increases of the HMW species for 3h56-269-IgG1a-C220S,C226A,C229A,P238S and 3h56-269-IgG1a-C220S,C226A,C229A,P238K compared to 3h56-269-IgG1a-C220S,P238K and 3h56-269-IgG1f-C220S,N297A. The increases in HMW were similar for 3h56-269-IgG1a-C220S,P238K and 3h56-269-IgG1f-C220S,N297A under each of the three temperatures tested.

Additional control dAb-Fc molecules were generated with altered and enhanced FcgR binding properties, including those with a wild type IgG1f Fc domain (3h56-269-IgG1f), or with additional point mutations to enhance binding to hCD32a-R131 and hCD32b (3h56-269-IgG1-S267E) or enhance specificity for hCD32b (3h56-269-IgG1f-G237D,P238D,H268D,P271G,A330R, also called 3h56-269-IgG1-V11). See Sequences 131-133 in Table 35.

TABLE 35

| SEQ | | | |
|---|---|---|---|
| ID | | | |
| NO: | Long name | Short ID | Sequence |
| 131 | 3h56-269- | 3h56- | EVQLLESGGGLVQPGGSLRLSCAASGFTFRDYEMWWVRQ |
| | IgG1f | 269- | APGKGLERVSAINPQGTRTYYADSVKGRFTISRDNSKNTLY |
| | | IgG1f | LQMNSLRAEDTAVYYCAKLPFRFSDRGQGTLVTVSSASTEP |
| | | | KSCDKTHTCPPCPAPELLGGPSVFLFPPKPKDTLMISRTPEV |
| | | | TCVVVDVSHEDPEVKFNWYVDGVEVHNAKTKPREEQYNS |
| | | | TYRVVSVLTVLHQDWLNGKEYKCKVSNKALPAPIEKTISK |
| | | | AKGQPREPQVYTLPPSREEMTKNQVSLTCLVKGFYPSDIAV |
| | | | EWESNGQPENNYKTTPPVLDSDGSFFLYSKLTVDKSRWQQ |
| | | | GNVFSCSVMHEALHNHYTQKSLSLSPGK |
| 132 | 3h56-269- | 3h56- | EVQLLESGGGLVQPGGSLRLSCAASGFTFRDYEMWWVRQ |
| | IgG1- | 269-SE | APGKGLERVSAINPQGTRTYYADSVKGRFTISRDNSKNTLY |
| | S267E | | LQMNSLRAEDTAVYYCAKLPFRFSDRGQGTLVTVSSASTEP |
| | | | KSCDKTHTCPPCPAPELLGGPSVFLFPPKPKDTLMISRTPEV |
| | | | TCVVVDVEHEDPEVKFNWYVDGVEVHNAKTKPREEQYNS |
| | | | TYRVVSVLTVLHQDWLNGKEYKCKVSNKALPAPIEKTISK |
| | | | AKGQPREPQVYTLPPSREEMTKNQVSLTCLVKGFYPSDIAV |
| | | | EWESNGQPENNYKTTPPVLDSDGSFFLYSKLTVDKSRWQQ |
| | | | GNVFSCSVMHEALHNHYTQKSLSLSPGK |
| 133 | 3h56-269- | 3h56- | EVQLLESGGGLVQPGGSLRLSCAASGFTFRDYEMWWVRQ |
| | IgG1f- | 269-V11 | APGKGLERVSAINPQGTRTYYADSVKGRFTISRDNSKNTLY |
| | G237D, P238D, | | LQMNSLRAEDTAVYYCAKLPFRFSDRGQGTLVTVSSASTEP |
| | H268D, | | KSCDKTHTCPPCPAPELLGDDSVFLFPPKPKDTLMISRTPEV |
| | P271G, A330R | | TCVVVDVSDEDGEVKFNWYVDGVEVHNAKTKPREEQYNS |
| | | | TYRVVSVLTVLHQDWLNGKEYKCKVSNKALPRPIEKTISK |
| | | | AKGQPREPQVYTLPPSREEMTKNQVSLTCLVKGFYPSDIAV |
| | | | EWESNGQPENNYKTTPPVLDSDGSFFLYSKLTVDKSRWQQ |
| | | | GNVFSCSVMHEALHNHYTQKSLSLSPGK |

These dAb-Fcs were tested for binding to human FcgRs using SPR, and the data demonstrated expected binding specificities. See Table 36.

TABLE 36
% Rmax data for 1 μM dAb-Fcs binding to anti-His Fab captured hFcgR-His proteins.

| | Anti-His Fab captured FcgR | | | | | |
|---|---|---|---|---|---|---|
| | High | | | | | |
| | affinity | Low Affinity FcgR | | | | |
| | FcgR | hCD32a- | hCD32a- | | hCD16a- | hCD16B- |
| Sample | hCD64 | H131 | R131 | hCD32b | V158 | NA2 |
| 3h56-269-IgG1f | 69% | 66% | 57% | 23% | 59% | 35% |
| 3h56-269-IgG1-S267E | 70% | 65% | 83% | 73% | 45% | 19% |
| 3h56-269-IgG1f-G237D, | 48% | 17% | 62% | 77% | 4% | 3% |
| P238D, H268D, P271G, | | | | | | |
| A330R | | | | | | |

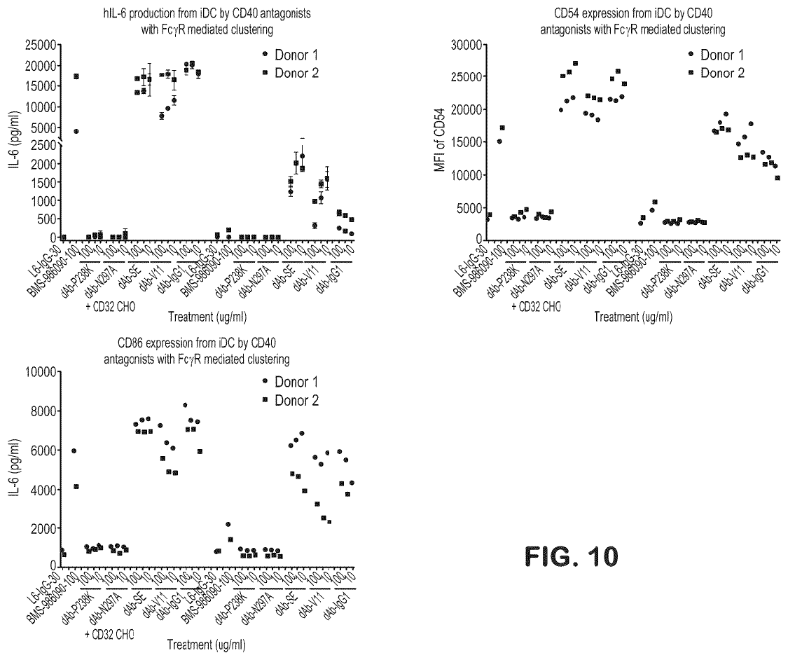

The iDC activation data for 3h-59-269-IgG1-V11, 3h-59-269-S267E show robust iDC activation at all concentrations tested in both the absence and presence of CD32-expressing CHO cells. See FIG. 10. The ability to tune immune cell activation is demonstrated by the activity of the 3h56-269-IgG1f fusion, which shows only modest activation in the absence of CD32 mediated cross-linking, which is then increased with CD32-overexpressing CHO cells. See FIG. 10.

Although the present embodiments have been described in detail with reference to examples above, it is understood that various modifications can be made without departing from the spirit of these embodiments, and would be readily known to the skilled artisan.

What is claimed is:

1. A human IgG1 Fc domain polypeptide comprising a mutation at Kabat position 238 that reduces binding to FC-gamma-receptors, wherein proline 238 (P238) is mutated to lysine.

2. An antibody polypeptide comprising:
(1) a single variable domain, said single variable domain comprising:
(a) a CDR1 region comprising the amino acid sequence of SEQ ID NO: 1 or differing from the CDR1 region of SEQ ID NO: 1 by up to two amino acids,
(b) a CDR2 region comprising the amino acid sequence of SEQ ID NO: 2 or differing from the CDR1 region of SEQ ID NO: 2 by up to three amino acids, and
(c) a CDR3 region comprising the amino acid sequence of SEQ ID NO: 3 or differing from the CDR1 region of SEQ ID NO: 3 by up to six amino acids, and wherein said single variable domain binds CD40; and
(2) the human IgG1 Fc domain according to claim 1.

3. The antibody polypeptide according to claim 2, wherein said single variable domain antagonize activities of CD40.

4. The antibody polypeptide according to claim 2, wherein said antibody polypeptide has increased stability.

5. The antibody polypeptide according to claim 2, wherein:
(a) the CDR1 region consists of the amino acid sequence of SEQ ID NO: 1 (CDR1 of 3h-56-269),
(b) the CDR2 region consists of the amino acid sequence of SEQ ID NO: 2 (CDR2 of 3h-56-269), and
(c) the CDR3 region consists of the amino acid sequence of SEQ ID NO: 3 (CDR3 of 3h-56-269).

6. The antibody polypeptide according to claim 2, wherein the amino acid sequence of the single variable domain is set forth in SEQ ID NO: 41 (3h-56-269 sequence).

7. A nucleic acid encoding the polypeptide of claim 5.

8. An expression vector comprising the nucleic acid molecule of claim 7.

9. A cell transformed with the expression vector of claim 8.

10. A pharmaceutical composition comprising the antibody polypeptide of claim 2, and a pharmaceutically acceptable carrier.

11. A method of treating or preventing an immune disease in a subject comprising administering to the subject the antibody polypeptide of claim 2.

12. The method according to claim 11, wherein the immune disease is selected from the group consisting of Addison's disease, allergies, anaphylaxis, ankylosing spondylitis, asthma, atherosclerosis, atopic allergy, autoimmune diseases of the ear, autoimmune diseases of the eye, autoimmune hepatitis, autoimmune parotitis, bronchial asthma, coronary heart disease, Crohn's disease, diabetes, epididymitis, glomerulonephritis, Graves' disease, Guillain-Barre syndrome, Hashimoto's disease, hemolytic anemia, idiopathic thrombocytopenic purpura, inflammatory bowel disease, immune response to recombinant drug products (e.g., Factor VII in hemophiliacs), systemic lupus erythematosus, multiple sclerosis, myasthenia gravis, pemphigus, psoriasis, rheumatic fever, rheumatoid arthritis, sarcoidosis, scleroderma, Sjogren's syndrome, spondyloarthropathies, thyroiditis, transplant rejection, vasculitis, and ulcerative colitis.
